SEQUENCE LISTING (4 sequences)

SEQ ID NO: 1 - DNA, 2016 bp, Homo sapiens
atgacaatccgacaccaaggccagcagtacaggccgaggatggcatttctccaaaagattgaagcgctcgtgaaggacatgcagaacccagagacaggggtccgaatgcagaaccagagggtcctggtcaccagcgttcctcatgccatgacaggaagtgatgttctgcaatggatcgtccagcggctttggatctccagtctggaggcacagaacttgggcaactttattgtcaggtatggctacatttaccccctgcaagaccccaagaatctcattctcaagcctgatggcagcctctacagatttcagacaccgtatttctggcccacccagcagtggccagctgaagataccgattacgccatctatctggccaagcgaaatatcaaaaagaaagggattttggaagaatatgaaaaggaaaattacaatttcttgaaccaaaaaatgaactataagtgggactttgtcattatgcaggccaaagagcagtacagggctggaaaggagaggaacaaagcagacagatatgccctggactgccaggagaaggcatactggctggtgcaccgatgccctcctggaatggacaatgtgctggactacggcctggaccgagtgaccaatccgaatgaagtcaagaaacaaacagtcgttgctgtcaaaaaagagatcatgtattaccaacaggccttgatgaggtccacagtgaagtcttctgtgtccctgggagggattgtgaaatacagtgagcagttctcatccaacgatgccatcatgtcaggctgcctccccagcaacccctggatcaccgatgacacccagttctgggacttaaatgccaaattggtggaaatcccaaccaagatgcgggtggaacgatgggccttcaacttcagcgaattgatccgagaccccaaaggtcgacagagcttccagtacttcctcaagaaagaattcagtggagagaatctgggattctgggaagcctgcgaggatctgaagtatggagatcagtccaaagtcaaggagaaagcagaggagatttacaagctgttcctggccccgggggcgaggcgctggatcaacatagatggcaaaaccatggacatcacagtgaaggggctgaagcacccccaccgctatgtgctggacgccgcacaaacccacatttacatgctcatgaagaaggattcttatgctcgctatttaaaatctccgatctataaggacatgctggccaaagctattgaacctcaggaaaccaccaagaaaagctccaccctcccttttatgcggcgtcacctgcgctccagcccaagccctgtcatcctgagacagctggaagaggaagccaaggcccgagaagcagccaacactgtggacatcacccagccgggccagcacatggctcccagcccccatctgaccgtgtacaccgggacctgcatgcccccgtctccttctagccccttctcctcctcctgccgctcccccaggaagcctttcgcctcacccagccgcttcatccggcgacccagcaccaccatctgcccctcacccatcagagtggccttggagagctcatcgggcttggagcagaaaggggagtgcagcgggtccatggccccccgtgggccctctgtcaccgagagcagcgaggcctccctcgacacctcctggcctcgcagccggcccagggcccctcctaaggcccgcatggctctgtccttcagcaggtttctgagacgaggctgtctggcctcacctgtctttgccaggctctcacccaagtgccctgctgtgtcccacgggagggtgcagcccctgggggacgtgggccagcagctgccacgattgaaatccaagagagtagcaaactttttccagatcaaaatggatgtgcccacggggagcgggacctgcttgatggactcggaggatgctggaacaggagagtcgggtgaccgggccacagaaaaggaggtcatctgcccctgggagagcctgtaa

SEQ ID NO: 2 - protein, 671 aa, Homo sapiens
MTIRHQGQQYRPRMAFLQKIEALVKDMQNPETGVRMQNQRVLVTSVPHAMTGSDVLQWIVQRLWISSLEAQNLGNFIVRYGYIYPLQDPKNLILKPDGSLYRFQTPYFWPTQQWPAEDTDYAIYLAKRNIKKKGILEEYEKENYNFLNQKMNYKWDFVIMQAKEQYRAGKERNKADRYALDCQEKAYWLVHRCPPGMDNVLDYGLDRVTNPNEVKKQTVVAVKKEIMYYQQALMRSTVKSSVSLGGIVKYSEQFSSNDAIMSGCLPSNPWITDDTQFWDLNAKLVEIPTKMRVERWAFNFSELIRDPKGRQSFQYFLKKEFSGENLGFWEACEDLKYGDQSKVKEKAEEIYKLFLAPGARRWINIDGKTMDITVKGLKHPHRYVLDAAQTHIYMLMKKDSYARYLKSPIYKDMLAKAIEPQETTKKSSTLPFMRRHLRSSPSPVILRQLEEEAKAREAANTVDITQPGQHMAPSPHLTVYTGTCMPPSPSSPFSSSCRSPRKPFASPSRFIRRPSTTICPSPIRVALESSSGLEQKGECSGSMAPRGPSVTESSEASLDTSWPRSRPRAPPKARMALSFSRFLRRGCLASPVFARLSPKCPAVSHGRVQPLGDVGQQLPRLKSKRVANFFQIKMDVPTGSGTCLMDSEDAGTGESGDRATEKEVICPWESL

SEQ ID NO: 3 - DNA, 570 bp, Homo sapiens
atggccctggtgaggggcggctggctgtggagacagagctccatcctccgccgctggaagcggaactggtttgccctgtggctggacgggaccctgggatactaccacgatgagacagcgcaggacgaggaggaccgtgtgctcatccacttcaatgtccgtgacataaagatcggcccagagtgccatgatgtgcagcccccagagggccggagccgagatggcctgctgactgtgaacctacgggaaggcggccgcctgcacctctgtgcggagaccaaggatgatgccctagcatggaagacagcactgctggaggcaaactccaccccggtgcgcgtctacagcccgtaccaagactactacgaggtggtgccccccaatgcacacgaggccacgtatgtccgcagctactacggaccgccctacgcaggccctggcgtgacgcacgtgatagtgcgggaggatccctgctacagcgccggcgcccctctggccatgggcatgcttgcgggagccgccactggggcggcgctgggctcgctcatgtggtcgccttgctggttctga

SEQ ID NO: 4 - protein, 189 aa, Homo sapiens
MALVRGGWLWRQSSILRRWKRNWFALWLDGTLGYYHDETAQDEEDRVLIHFNVRDIKIGPECHDVQPPEGRSRDGLLTVNLREGGRLHLCAETKDDALAWKTALLEANSTPVRVYSPYQDYYEVVPPNAHEATYVRSYYGPPYAGPGVTHVIVREDPCYSAGAPLAMGMLAGAATGAALGSLMWSPCWF

(19) United States
(12) Patent Application Publication
(10) Pub. No.: US 2003/0166850 A1
(43) Pub. Date: Sep. 4, 2003

(54) Novel RGS9 protein binding interactions and methods of use thereof

(75) Inventors: Philip G. Jones, Cranbury, NJ (US); Kathleen H. Young, Newtown, PA (US)

Correspondence Address: WYETH, PATENT LAW GROUP, FIVE GIRALDA FARMS, MADISON, NJ, 07940, US

(73) Assignee: Wyeth, Madison, NJ

(21) Appl. No.: 10/108,210

(22) Filed: Mar. 27, 2002

Related U.S. Application Data
(60) Provisional application No. 60/279,240, filed on Mar. 28, 2001.

(51) Int. Cl. C07K 1/00; C07K 14/00; C07K 17/00

(52) U.S. Cl. 530/350

(57) ABSTRACT

The present invention relates to novel protein binding interactions, comprising a regulator of G-protein signalling protein (RGS) and a non G-protein binding partner. More particularly, the invention relates to a novel interaction between RGS9 and evectin polypeptides, the use of such polypeptides, as well as the production of such polypeptides. The invention relates also to identifying compounds which may be agonists, antagonists and/or inhibitors of RGS9 and/or evectin polypeptides, and therefore potentially useful in therapy. In particular embodiments, the RGS9 and evectin polypeptides produced are used in methods for assaying the effects of test compounds on the activity of RGS9-evectin dimers, methods for assaying the effects of test compounds on the activity of RGS9-evectin dimers comprised in transgenic animals encoding RGS9 and evectin, methods for diagnosis and treatment of diseases related to the activity of RGS9-evectin dimers and methods for modulating G-protein activity.

### FIELD OF THE INVENTION

The present invention relates generally to the fields of cell signaling, neuroscience and molecular biology. More particularly, the invention relates to newly identified protein binding interactions, comprising a regulator of G-protein signaling (RGS) protein and a non G-protein binding partner, the use of such polypeptides, as well as the production of such polypeptides. The invention relates also to identifying compounds which may be agonists, antagonists and/or inhibitors of the RGS-binding partner interaction, and therefore potentially useful in therapy.

### BACKGROUND OF THE INVENTION

The heterotrimeric guanine nucleotide binding proteins (G-proteins) are intracellular proteins best known for their role as transducers of binding by extracellular ligands to seven transmembrane receptors (7-TMRs) located on the cell surface. Individual 7-TMRs have been identified for many small neurotransmitters (e.g. adrenaline, noradrenaline, dopamine, serotonin, histamine, acetylcholine, GABA, glutamate, and adenosine), for a variety of neuropeptides and hormones (e.g. opioids, tachykinins, bradykinins, vasoactive intestinal peptide, neuropeptide Y, thyrotrophic hormone, leutenizing hormone, follicle-stimulating hormone, adrenocorticotropic hormone, cholecystokinin, gastrin, glucagon, somatostatin, endothelin, vasopressin and oxytocin) as well as for chemoattractant chemokines (C5a, interleukin-8, platelet-activating factor and the N-formyl peptides) that are involved in immune function. In addition, the odorant receptors present on vertebrate olfactory cells are 7-TMRs, as are rhodopsins, the proteins that transduce visual signals.

Ligand binding to 7-TMRs produces activation of one or more heterotrimeric G-proteins. A few proteins with structures that are dissimilar to the 7-TMRs also have been shown to activate heterotrimeric G-proteins. These include the amyloid precursor protein (APP), the terminal complement complex, the insulin-like growth factor/mannose 6-phosphate receptor and the ubiquitous brain protein GAP-43. Dysregulation of G-protein coupled pathways is associated with a wide variety of diseases, including diabetes, hyperplasia, psychiatric disorders, cardiovascular disease, and possibly Alzheimer's disease. Accordingly, the 7-TMRs are targets for a large number of therapeutic drugs: for example, the β-adrenergic blockers used to treat hypertension target 7-TMRS.

Unactivated heterotrimeric G-proteins are complexes comprised of three subunits, Gα, Gβ and Gγ. The subunits are encoded by three families of genes: in mammals there are at least 17 Gα, 5 Gβ and 11 Gγ genes. Additional diversity is generated by alternate splicing. Where it has been studied, a similar multiplicity of G-proteins has been found in invertebrate animals. Mutations within Gα subunit genes is involved in the pathophysiology of several human diseases: mutations of Gα that activate Gs or Gi2 are observed in some endocrine tumors and are responsible for McCune-Albright syndrome, whereas loss-of-function mutations of Gα are found in Albright hereditary osteodystrophy.

The Gα subunits have binding sites for a guanine nucleotide and intrinsic GTPase activity. Prior to activation the complex contains bound GDP: GαGDPβγ. Activation involves the receptor catalyzed release of GDP followed by binding of GTP and concurrent dissociation of the complex into two signalling complexes: GαGTP and βγ. Signaling through GαGTP is terminated by GTP hydrolysis to GDP by the intrinsic GTPase activity of the Gα subunit. GαGDP then reassociates with βγ to reform the inactive, heterotrimeric complex.

The mammalian G-proteins are divided into four subtypes: Gs, Gi/Go, Gq and G12. This typing is based on the effect of activated G-proteins on enzymes that generate second messengers, on their sensitivity to cholera and pertussis toxin and their sequence. These divisions also appear to be evolutionarily ancient: there are comparable subtypes in invertebrate animals. Members of two subtypes of G-proteins control the activity of adenylyl cyclases (ACs), enzymes responsible for the synthesis of cyclic adenosine monophosphate (cAMP). cAMP is a diffusible second messenger that acts through cAMP-dependent protein kinases (PKAs) to phosphorylate a large number of target proteins. Activated Gs proteins increase the activity of ACs whereas activated Gi proteins inhibit these enzymes. Gs proteins are also uniquely activated by cholera toxin whereas, activation of Gi and Go are blocked by pertussis toxin. The Gq Gα subunits increase the activity of inositol phospholipid-specific phospholipases (IP-PLCs) and furthermore the βy dimer of Gi/o heterotrimers can also stimulate PLCβ2. IP-PLCs release two diffusible second messengers, inositol triphosphate (IP₃) and diacylglycerol (DAG). IP₃ modulates intracellular Ca²⁺ concentration, whereas DAG activates protein kinase Cs (PKCs) to phosphorylate many target proteins. The second messenger cascades allow signals generated by G-protein activation to have global effects on cellular physiology.

Activation of G-proteins frequently modulate ion conductance through plasma membrane ion channels. Although in some cases these effects are indirect, as a result of changes in second messengers, G-proteins can also couple directly to ion channels. This phenomenon is known as membrane delimited modulation. The opening of inwardly rectifying K channels by activated Gi/Go and of N and L type Ca channels by Gi/Go and Gq are commonly observed forms of membrane delimited modulation.

Heterotrimeric G-proteins appear to have other cellular roles, in addition to transducing the binding of extracellular ligands. Analysis of the intracellular localization of the various G-protein subunits combined with pharmacological studies suggest, for example, that G-proteins are involved in intracellular membrane trafficking. Indeed, some workers hypothesize that G-proteins evolved to control membrane trafficking and that their role in transducing extracellular signals evolved later. Studies implicate heterotrimeric G-proteins in the formation of vesicles from the trans-Golgi network, in transcytosis in polarized epithelial cells and in the control of secretion in many cells, including several model systems relevant to human disease: mast cells, chromaffin cells of the adrenal medulla and human airway epithelial cells. Nonetheless, the G-protein subunits involved in membrane trafficking and secretion have yet to be definitively established and the mechanisms by which they are activated and control membrane trafficking remains largely unknown.

It is well established that many medically significant biological processes are mediated by polypeptides participating in cellular signal transduction pathways that involve G-proteins and second messengers, e.g., cAMP, IP₃ and diacylglycerol (Lefkowitz, 1991). Thus, there is clearly a need for the identification and characterization of further proteins, their genes and their ligands, which can play a role in preventing, ameliorating or correcting dysfunctions or diseases related to cellular signaling.

### SUMMARY OF THE INVENTION

The present invention broadly relates to newly identified protein binding interactions, comprising a regulator of G-protein signaling protein (RGS) and a non G-protein binding partner. More particularly, the invention relates to a novel interaction between RGS9 and evectin polypeptides, the use of such polypeptides, as well as the production of such polypeptides. The invention relates also to identifying compounds which may be agonists, antagonists and/or inhibitors of the RGS-evectin interaction, and therefore potentially useful in therapy.

In particular embodiments, the invention is directed to an isolated human RGS9 polypeptide fragment comprising an evectin polypeptide binding domain, wherein the RGS9 polypeptide fragment comprises the amino acid sequence from amino acid 461 through amino acid 602 of SEQ ID NO:2. In another embodiment, an isolated human evectin polypeptide fragment is provided, comprising a RGS9 polypeptide binding domain, wherein the evectin polypeptide fragment comprises the amino acid sequence from amino acid 79 through amino acid 136 of SEQ ID NO:4. In particular embodiments, an isolated polynucleotide encoding the RGS9 polypeptide fragment is provided, comprising the evectin binding domain, wherein the polynucleotide comprises the nucleotide sequence of SEQ ID NO:1 and an isolated polynucleotide encoding the evectin polypeptide fragment is provided, comprising the RGS9 binding domain, wherein the polynucleotide comprises the nucleotide sequence of SEQ ID NO:3, or a degenerate variant thereof.

In another embodiment, the invention is directed to an isolated human RGS9-evectin polypeptide dimer. In a preferred embodiment, the dimer comprises a RGS9 polypeptide comprising the amino acid sequence of SEQ ID NO:2 and a evectin polypeptide comprising the amino acid sequence of SEQ ID NO:4. In yet another preferred embodiment, the RGS9 polypeptide of SEQ ID NO:2 is encoded by a polynucleotide comprising the nucleotide sequence of SEQ ID NO:1, or a degenerate variant thereof and the evectin polypeptide of SEQ ID NO:4 is encoded by a polynucleotide comprising the nucleotide sequence of SEQ ID NO:3, or a degenerate variant thereof.

In other embodiments, the invention is directed to an antibody specific for a RGS9-evectin dimer comprising a RGS9 polypeptide of SEQ ID NO:2 and a evectin polypeptide of SEQ ID NO:4. In another embodiment, an antibody specific for an RGS9 polypeptide fragment comprising the amino acid sequence from amino acid 461 through amino acid 602 of SEQ ID NO:2 is provided. In yet another embodiment, an antibody specific for an evectin polypeptide fragment comprising the amino acid sequence from amino acid 79 through amino acid 136 of SEQ ID NO:4 is provided.

The present invention further provides in certain embodiments, a transgenic animal whose genome comprises an engineered functional disruption in the polynucleotide encoding the endogenous RGS9 polypeptide, wherein the disruption occurs in the evectin binding domain of the RGS9 polypeptide. In particular embodiments, this animal is homozygous for the functional disruption. Provided also is a transgenic animal whose genome comprises an engineered functional disruption in the polynucleotide encoding the endogenous evectin polypeptide, wherein the disruption occurs in the RGS9 binding domain of the evectin polypeptide. In particular embodiments, this animal is homozygous for the functional disruption.

In certain embodiments, the invention is directed to a recombinant expression vector comprising a polynucleotide encoding a human RGS9 polypeptide fragment comprising an evectin polypeptide binding domain, wherein the RGS9 fragment comprises the amino acid sequence from amino acid 461 through amino acid 602 of SEQ ID NO:2. In certain other embodiments, the invention is directed to a recombinant expression vector comprising a polynucleotide encoding a human evectin polypeptide fragment comprising a RGS9 polypeptide binding domain, wherein the evectin fragment comprises the amino acid sequence from amino acid 79 through amino acid 136 of SEQ ID NO:4. Further provided is a genetically engineered host cell, transfected, transformed or infected with a one of the above recombinant expression vectors. In a preferred embodiment, the host cell is a bacterial cell. In other embodiments, the host cell is selected from the group consisting of a yeast cell, an insect cell, a plant cell and an animal cell.

In another embodiment of the invention, a DNA chip is provided comprising an array of polynucleotides, wherein at least one of the polynucleotides comprises a nucleotide sequence encoding an RGS9 polypeptide fragment comprising an evectin polypeptide binding domain, wherein the RGS9 polypeptide fragment comprises the amino acid sequence from amino acid 461 through amino acid 602 of SEQ ID NO:2 or a DNA chip comprising an array of polynucleotides, wherein at least one of the polynucleotides comprise a nucleotide sequence encoding an evectin polypeptide fragment comprising a RGS9 polypeptide binding domain, wherein the evectin polypeptide fragment comprises the amino acid sequence from amino acid 79 through amino acid 136 of SEQ ID NO:4.

In yet another embodiment, the invention is directed to a protein chip comprising an array of polypeptides, wherein at least one of the polypeptides comprises a RGS9 polypeptide fragment comprising an evectin polypeptide binding domain, wherein the RGS9 polypeptide fragment comprises the amino acid sequence from amino acid 461 through amino acid 602 of SEQ ID NO:2 or a protein chip comprising an array of polypeptides, wherein at least one of the polypeptides comprises an evectin polypeptide fragment comprising a RGS9 polypeptide binding domain, wherein the evectin polypeptide fragment comprises the amino acid sequence from amino acid 79 through amino acid 136 of SEQ ID NO:4.

The present invention further provides methods for assaying the effects of test compounds on the activity of RGS9-evectin polypeptide dimers. In one embodiment, the invention is directed to a method for assaying the effects of test compounds on the activity of a RGS9-evectin polypeptide dimer comprising the steps of providing recombinant cells comprising a RGS9 polypeptide having an amino acid sequence of SEQ ID NO:2 and an evectin polypeptide having an amino acid sequence of SEQ ID NO:4, contacting the cells with a test compound and determining the effect of the test compound on the activity of the RGS9-evectin dimer in the presence and absence of the test compound. In one embodiment, the RGS9 polypeptide comprises at least one mutation within the evectin binding domain, wherein the evectin binding domain comprises amino acid 461 through amino acid 602 of SEQ ID NO:2. In another embodiment, the evectin polypeptide comprises at least one mutation within the RGS9 binding domain, wherein the RGS9 binding domain comprises amino acid 79 through amino acid 136 of SEQ ID NO:4. In a preferred embodiment, the activity of the RGS9-evectin polypeptide is detected by a G-protein second messenger response selected from the group consisting of an inositol triphosphate/diacyl glycerol-protein kinase C system, an adenylate cyclase/cyclic AMP-dependent protein kinase system, a guanylate cyclase/cGMP dependent protein kinase system or an ion channel.

In another embodiment, a method is provided for assaying the effects of test compounds on the activity of a RGS9-evectin polypeptide dimer comprising the steps of providing a transgenic animal comprising a polynucleotide encoding a RGS9 polypeptide having an amino acid sequence of SEQ ID NO:2 and a polynucleotide encoding an evectin polypeptide having an amino acid sequence of SEQ ID NO:4, administering a test compound to the animal and determining the effects of the test compound on the activity of the RGS9-evectin polypeptide in the presence and absence of the test compound. In certain embodiments, the polynucleotide encoding the RGS9 polypeptide has at least one mutation within the evectin binding domain, wherein the evectin binding domain comprises amino acid 461 through amino acid 602 of SEQ ID NO:2. In certain other embodiments, the polynucleotide encoding the evectin polypeptide has at least one mutation within the RGS9 binding domain, wherein the RGS9 binding domain comprises amino acid 79 through amino acid 136 of SEQ ID NO:4.

In certain other embodiments of the invention, a method is provided for assaying the effects of test compounds on a transgenic animal with a genome comprising a functional disruption of the evectin binding domain in the RGS9 polypeptide, the method comprising providing a transgenic animal whose genome comprises a disruption of the endogenous polynucleotide encoding the RGS9 polypeptide, wherein the disruption occurs in the evectin binding domain, administering a test compound to the animal and determining the effects of the test compound on the activity of the RGS9 polypeptide in the presence and absence of the test compound.

In yet another embodiment, provided is a method for assaying the effects of test compounds on a transgenic animal with a genome comprising a functional disruption of the RGS9 binding domain in the evectin polypeptide, the method comprising providing a transgenic animal whose genome comprises a disruption of the endogenous polynucleotide encoding the evectin polypeptide, wherein the disruption occurs in the RGS9 binding domain, administering a test compound to the animal and determining the effects of the test compound on the activity of the evectin polypeptide in the presence and absence of the test compound.

Another embodiment provides a method for assaying the effects of test compounds on the binding interaction of RGS9 and evectin polypeptides comprising the steps of providing yeast cells for a yeast two-hybrid system comprising a RGS9 polypeptide having an amino acid sequence of SEQ ID NO:2 and an evectin polypeptide having an amino acid sequence of SEQ ID NO:4, contacting the cells with a test compound and determining the effect of the test compound on the binding interaction of the RGS9 and evectin polypeptides in the presence and absence of the test compound.

In one embodiment, the invention is directed to a method for producing a transgenic animal whose genome comprises a functional disruption of the evectin binding domain in a polynucleotide encoding a RGS9 polypeptide, the method comprising providing a polynucleotide encoding a RGS9 polypeptide having a functional disruption in the evectin binding domain, wherein the binding domain comprises the amino acid sequence of amino acid 461 through amino acid 602 of SEQ ID NO:2, introducing the disrupted polynucleotide into embryonic stem cells, selecting those embryonic stem cells that comprise the disrupted polynucleotide, introducing the embryonic stem cell into a blastocyst, transferring the blastocyst to a pseudopregnant animal and allowing the transferred blastocyst to develop into an animal chimeric for the disruption. In another embodiment, the method further comprises breeding the chimeric animal with a wild-type animal to obtain animals heterozygous for the disruption. In yet another embodiment, the method further comprises breeding the heterozygous animal to generate animal homozygous for the disruption.

In yet other embodiment, the invention is directed to a method for producing a transgenic animal whose genome comprises a functional disruption of the RGS9 binding domain in a polynucleotide encoding an evectin polypeptide, the method comprising providing a polynucleotide encoding an evectin polypeptide having a functional disruption in the RGS9 binding domain, wherein the binding domain comprises the amino acid sequence of amino acid 79 through amino acid 136 of SEQ ID NO:4, introducing the disrupted polynucleotide into embryonic stem cells, selecting those embryonic stem cells that comprise the disrupted polynucleotide, introducing the embryonic stem cell into a blastocyst, transferring the blastocyst to a pseudopregnant animal and allowing the transferred blastocyst to develop into an animal chimeric for the disruption. In another embodiment, the method further comprises breeding the chimeric animal with a wild-type animal to obtain animals heterozygous for the disruption. In yet another of the embodiments, the method further comprises breeding the heterozygous animal to generate animal homozygous for the disruption.

The invention is directed in other embodiments to a method for modulating G-protein activity in a subject comprising administering to the subject a therapeutically effective amount of a RGS9 polypeptide fragment comprising an evectin polypeptide binding domain, wherein the RGS9 polypeptide fragment comprises the amino acid sequence from amino acid 461 through amino acid 602 of SEQ ID NO:2, or administering to the subject a therapeutically effective amount of an evectin polypeptide fragment, comprising a RGS9 polypeptide binding domain, wherein the evectin polypeptide fragment comprises the amino acid sequence from amino acid 79 through amino acid 136 of SEQ ID NO:4

In certain other embodiments, the invention provides a method for modulating G-protein activity in a subject comprising administering to the subject a therapeutically effective amount of a polynucleotide antisense to a polynucleotide encoding the RGS9 polypeptide fragment comprising the evectin binding domain, wherein the polynucleotide comprises the nucleotide sequence of SEQ ID NO:1 or administering to the subject a therapeutically effective amount of a polynucleotide antisense to a polynucleotide encoding the evectin polypeptide fragment comprising the RGS9 binding domain, wherein the polynucleotide comprises the nucleotide sequence of SEQ ID NO:3.

In certain other embodiments, the invention is directed to a method for modulating G-protein activity in a subject comprising administering to the subject a therapeutically effective amount of an antibody specific for a RGS9-evectin dimer comprising a RGS9 polypeptide of SEQ ID NO:2 and a evectin polypeptide of SEQ ID NO:4, or administering to the subject a therapeutically effective amount of an antibody specific for an RGS9 polypeptide fragment comprising the amino acid sequence from amino acid 461 through amino acid 602 of SEQ ID NO:2, or administering to the subject a therapeutically effective amount of an antibody specific for an evectin polypeptide fragment comprising the amino acid sequence from amino acid 79 through amino acid 136 of SEQ ID NO:4.

In certain embodiments, the invention is directed to a method for modulating G-protein activity in a subject comprising administering to the subject a therapeutically effective amount of an expression vector comprising a polynucleotide encoding a human RGS9 polypeptide fragment comprising an evectin polypeptide binding domain, wherein the RGS9 fragment comprises the amino acid sequence from amino acid 461 through amino acid 602 of SEQ ID NO:2 or a recombinant expression vector comprising a polynucleotide encoding a human evectin polypeptide fragment comprising a RGS9 polypeptide binding domain, wherein the evectin fragment comprises the amino acid sequence from amino acid 79 through amino acid 136 of SEQ ID NO:4.

In a particular embodiment, a method is provided for the diagnosis of a disease or susceptibility to a disease in a subject related to the activity of a RGS9-evectin dimer, the method comprising obtaining a biological sample from the subject, contacting the sample with an oligonucleotide probe of a polynucleotide encoding an RGS9 polypeptide fragment and an oligonucleotide probe of a polynucleotide encoding an evectin polypeptide fragment under stringent hybridization conditions, isolating the hybrids from the sample and sequencing the hybrids; wherein a mutation in the RGS9 and/or evectin polynucleotide sequence indicates a disease or susceptibility to a disease related to the activity of a RGS9-evectin dimer. In a preferred embodiment, the disease is a neurological disorder.

Other embodiments are directed to a method for the diagnosis of a disease or susceptibility to a disease in a subject related to the activity of a RGS9-evectin dimer, the method comprising obtaining a biological sample from the subject, contacting the sample with an oligonucleotide primer of a polynucleotide encoding an RGS9 polypeptide fragment and an oligonucleotide primer of a polynucleotide encoding an evectin polypeptide fragment, in the presence of nucleotides and a polymerase enzyme under conditions permitting primer extension, isolating primer extension products in the sample and sequencing the primer extension products, wherein a mutation in the RGS9 and/or evectin polynucleotide sequence indicates a disease or susceptibility to a disease related to the activity of a RGS9-evectin dimer. In particular embodiments, the disease is a neurological disorder.

In still other embodiments, the invention is directed to a method for the diagnosis of disease or susceptibility to a disease in a subject related to the activity of a RGS9-evectin dimer, the method comprising obtaining a biological sample from the subject; contacting the sample with an antibody specific for a RGS9-evectin polypeptide dimer; detecting the presence of an antibody-RGS9-evectin polypeptide complex, isolating the antibody-RGS9-evectin polypeptide complex, separating the antibody from the RGS9-evectin polypeptide and assaying the activity of the RGS9-evectin polypeptide, wherein an increased activity or a decreased activity of the RGS9-evectin polypeptide dimer relative to a control RGS9-evectin polypeptide dimer, indicates a disease or susceptibility to a disease related to the activity of a RGS9-evectin dimer. In particular embodiments, the disease is a neurological disorder.

Still further embodiments are directed to a method for the treatment of a subject in need of enhanced RGS9-evectin dimer activity comprising administering to the subject a therapeutically effective amount of an agonist to the RGS9-evectin and/or administering to the subject a polynucleotide encoding a RGS9-evectin polypeptide, in a form so as to effect the production of the RGS9-evectin activity in vivo.

Further embodiments are directed to a method for the treatment of a subject in need of inhibiting RGS9-evectin dimer activity comprising administering to the subject a therapeutically effective amount of an antagonist to the RGS9-evectin and/or administering to the subject a polynucleotide that inhibits the expression of a polynucleotide encoding a RGS9-evectin polypeptide; and/or administering to the subject a therapeutically effective amount of a polypeptide that competes with RGS9-evectin for its ligand.

Other features and advantages of the invention will be apparent from the following detailed description, from the preferred embodiments thereof, and from the claims.

### BRIEF DESCRIPTION OF THE DRAWINGS

FIGS. 1A and 1B. FIG. 1A, A schematic representation of the domain structure of evectin. Indicated on the diagram are the deletion mutants and the putative RGS9 binding domain identified using them. Also noted are the PH domain and a putative C terminal transmembrane domain. FIG. 1B, Schematic representation of the proline rich domain of RGS9-2 indicating the deletion mutants which were used to identify the putative evectin binding domain.

### DETAILED DESCRIPTION

Recently, a growing family of proteins termed Regulators of G-protein Signaling (RGS) proteins has emerged (Hepler, 1999; Ross and Wilkie, 2000). These proteins are known to increase the GTPase activity of G-protein α-subunits and therefore reduce the flux through the signaling pathway. The RGS family of proteins are characterized by a 120 amino acid RGS domain which is necessary, and in many cases sufficient, to stimulate the GTPase activity of G-protein α-subunits. It is contemplated in the present invention, that novel compounds which modulate RGS activity, will be therapeutically useful to control defects in cellular signaling. RGS inhibitors would increase the signaling through G-protein linked pathways and thus overcome any deficits, conversely an enhancement of RGS activity would decrease cellular signaling. To date there are close to 30 proteins which contain an RGS domain. These proteins are very diverse in structure and distribution. Some RGS proteins are relatively small and contain little more than the RGS domain, while others are more complex and contain multiple protein modules which have their own specific functions and/or bind particular proteins, thus serving to diversify the biological role of RGS proteins. The identification and understanding of these secondary interactions will facilitate the design of compounds developed to target a specific RGS protein or a particular function of an RGS protein.

The expression patterns of RGS proteins can give an indication of their potential role in the pathophysiology of neuronal signaling. Many of the RGS proteins have a discrete localization, one example being RGS9, which is almost exclusively localized to retina and brain (see Cowan et al., 2001). An alternatively spliced form of RGS9 (RGS9-2), containing a unique C-terminal proline rich domain, is found in the brain (Rahman et al., 1999). RGS9 is highly enriched in the striatum, a brain region associated with neurological disorders such as Parkinson's disease and schizophrenia. The discrete localization of RGS9-2, and its crucial role in the control of cellular signaling, makes it an attractive target for therapeutic intervention in such disorders. In addition, the role of RGS9 in both neuronal and visual signal transduction makes it necessary to target the brain specific functions of RGS9, since an inhibitor of the RGS domain would undoubtedly have visual side effects. It is likely that the neuronal specific functions are mediated in part, through the brain specific C-terminal, proline-rich domain.

Thus, to further investigate the role of RGS9-2 in striatal function, a yeast two hybrid assay was used to identify proteins interacting with this region. On screening a fetal brain library, the present invention has identified a protein called evectin 1 (Krappa et al., 1999), which is also known as PHR1 (Xu et al., 1999). Evectin 1 contains an N-terminal pleckstrin homology domain, a protein module commonly found in proteins of signal transduction complexes and which is known to interact with G-protein βγ subunits. At least 2 alternatively spliced forms of evectin exist (e.g., a long and short form) and it has been determined in the present invention, that only the shorter form of evectin, lacking exon 2, interacts with RGS9. Using a series of C-terminal deletion mutants, a domain (amino acids 79-136) in evectin 1 was identified which is required for the interaction with RGS9. In the longer form, this putative interacting domain is disrupted by a 35 amino acid insert encoded by exon 2, and this may account for its lack of interaction in the yeast assay. The evectin binding site in the RGS9 protein is located between amino acids (461-602) of the proline rich domain. It is contemplated here that the interaction of evectin 1 with RGS9 might be involved in modulating the activity of RGS9, localization of RGS9 or may impart a striatal specific function to RGS9. Thus, the modulation of one or more of these functions would be useful in therapeutic approaches for the treatment of disorders arising from aberrant striatal signaling.

The present invention has identified novel protein binding interactions, comprising a regulator of G-protein signalling protein (RGS) and a non G-protein binding partner. More particularly, the invention has identified a novel interaction between RGS9 and evectin polypeptides. In certain embodiments, the present invention provides polynucleotides useful in the production of RGS9 and evectin polypeptides or fragments thereof. In particular embodiments, the RGS9 and evectin polypeptides produced are used in methods for assaying the effects of test compounds on the activity of RGS9-evectin dimers, methods for assaying the effects of test compounds on the activity of RGS9-evectin dimers comprised in transgenic animals encoding RGS9 and evectin, methods for diagnosis and treatment of diseases related to the activity of RGS9-evectin dimers and methods for modulating G-protein activity. Additionally, the present invention provides RGS9 polypeptide fragments comprising an evectin polypeptide binding domain, wherein the RGS9 polypeptide fragment comprises the amino acid sequence from amino acid 461 through amino acid 602 of SEQ ID NO:2. In other embodiments, provided are evectin polypeptide fragments comprising a RGS9 polypeptide binding domain, wherein the evectin polypeptide fragment comprises the amino acid sequence from amino acid 79 through amino acid 136 of SEQ ID NO:4.

Compositions and methods for use of the polynucleotides, polypeptides, antibodies, expression vectors, host cells and transgenic animals of the present invention are discussed in the following sections.

A. Isolated Polynucleotides that Encode RGS9 and Evectin Polypeptides

Isolated and purified RGS9 and evectin polynucleotides of the present invention are contemplated for use in the production of RGS9 and evectin polypeptide dimers, RGS9 polypeptide fragments and evectin polypeptide fragments. In particular embodiments, the RGS9 and evectin polypeptides and fragments thereof are used in methods for assaying the effects of test compounds on the activity of RGS9-evectin dimers, methods for assaying the effects of test compounds on the activity of RGS9-evectin dimers comprised in transgenic animals encoding RGS9 and evectin, methods for diagnosis and treatment of diseases related to the activity of RGS9-evectin dimers and methods for modulating G-protein activity. In other embodiments, antibodies are provided specific for RGS9 polypeptide fragments, evectin polypeptide fragments, RGS9-evectin dimers and fragments thereof, transgenic animals comprising functional disruptions in a RGS9 and/or evectin binding domain, recombinant expression vectors encoding RGS9 polypeptide fragments, recombinant expression vectors encoding evectin polypeptide fragments, recombinant expression vectors encoding RGS9-evectin dimers, and host cells comprising these vectors.

Thus, in one aspect, the present invention provides isolated and purified polynucleotides that encode RGS9 and evectin polypeptides. In particular embodiments, a polynucleotide of the present invention is a DNA molecule. In a preferred embodiment, a polynucleotide of the present invention encodes an isolated human RGS9 polypeptide fragment comprising an evectin polypeptide binding domain, wherein the RGS9 polypeptide fragment comprises the amino acid sequence from amino acid 461 through amino acid 602 of SEQ ID NO:2. In another embodiment, a polynucleotide encodes an isolated human evectin polypeptide fragment comprising a RGS9 polypeptide binding domain, wherein the evectin polypeptide fragment comprises the amino acid sequence from amino acid 79 through amino acid 136 of SEQ ID NO:4. In particular embodiments, an isolated polynucleotide encoding the RGS9 polypeptide fragment comprising the evectin binding domain comprises the nucleotide sequence of SEQ ID NO:1, or a degenerate variant thereof and an isolated polynucleotide encoding the evectin polypeptide fragment comprising the RGS9 binding domain comprises the nucleotide sequence of SEQ ID NO:3, or a degenerate variant thereof. In a preferred embodiment, an isolated human RGS9-evectin polypeptide dimer is provided, wherein the RGS9 polypeptide comprises the amino acid sequence of SEQ ID NO:2 and the evectin polypeptide comprises the amino acid sequence of SEQ ID NO:4.

As used herein, the term “polynucleotide” means a sequence of nucleotides connected by phosphodiester linkages. Polynucleotides are presented herein in the direction from the 5′ to the 3′ direction. A polynucleotide of the present invention can comprise from about 40 to about several hundred thousand base pairs. Preferably, a polynucleotide comprises from about 10 to about 3,000 base pairs. Preferred lengths of particular polynucleotides are set forth hereinafter.

A polynucleotide of the present invention can be a deoxyribonucleic acid (DNA) molecule, a ribonucleic acid (RNA) molecule, or analogs of the DNA or RNA generated using nucleotide analogs. The nucleic acid molecule can be single-stranded or double-stranded, but preferably is double-stranded DNA. Where a polynucleotide is a DNA molecule, that molecule can be a gene, a cDNA molecule or a genomic DNA molecule. Nucleotide bases are indicated herein by a single letter code: adenine (A), guanine (G), thymine (T), cytosine (C), inosine (I) and uracil (U).

“Isolated” means altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated,” as the term is employed herein.

Preferably, an “isolated” polynucleotide is free of sequences which naturally flank the nucleic acid (i.e., sequences located at the 5′ and 3′ ends of the nucleic acid) in the genomic DNA of the organism from which the nucleic acid is derived. For example, in various embodiments, the isolated RGS9 and/or evectin nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived (e.g., neuronal or placenta). However, the RGS9 and/or evectin nucleic acid molecule can be fused to other protein encoding or regulatory sequences and still be considered isolated.

Polynucleotides of the present invention may be obtained, using standard cloning and screening techniques, from a cDNA library derived from mRNA from human cells or from genomic DNA. Polynucleotides of the invention can also be synthesized using well known and commercially available techniques.

The invention further encompasses nucleic acid molecules that differ from the nucleotide sequence of SEQ ID NO:1 encoding the RGS9 polypeptide fragment comprising an evectin polypeptide binding domain, wherein the RGS9 fragment comprises the amino acid sequence from amino acid 461 through amino acid 602 of SEQ ID NO:2, due to degeneracy of the genetic code and thus encode the same RGS9 polypeptide as that encoded by the nucleotide sequence shown in SEQ ID NO:1. Similarly, the invention encompasses nucleic acid molecules that differ from the nucleotide sequence of SEQ ID NO:3, encoding the evectin polypeptide fragment comprising a RGS9 polypeptide binding domain, wherein the evectin polypeptide fragment comprises the amino acid sequence from amino acid 79 through amino acid 136 of SEQ ID NO:4, due to degeneracy of the genetic code and thus encode the same evectin polypeptide as that encoded by the nucleotide sequence shown in SEQ ID NO:3.

In another preferred embodiment, an isolated polynucleotide of the invention comprises a nucleic acid molecule which is a complement of the nucleotide sequence shown in SEQ ID NO:1 or SEQ ID NO:3, or a fragment of these nucleotide sequences. A nucleic acid molecule which is complementary to the nucleotide sequence shown in SEQ ID NO:1 or SEQ ID NO:3 is one which is sufficiently complementary to the nucleotide sequence SEQ ID NO:1 or SEQ ID NO:3, such that it can hybridize to the nucleotide sequence shown in SEQ ID NO:1 or SEQ ID NO:3, thereby forming a stable duplex.

Orthologues and allelic variants of the human RGS9 and evectin polynucleotides can readily be identified using methods well known in the art. Allelic variants and orthologues of the RGS9 and evectin will comprise a nucleotide sequence that is typically at least about 70-75%, more typically at least about 80-85%, and most typically at least about 90-95% or more homologous to the nucleotide sequence shown in SEQ ID NO:1 or SEQ ID NO:3, or a fragment of these nucleotide sequences. Such nucleic acid molecules can readily be identified as being able to hybridize, preferably under stringent conditions, to the nucleotide sequence shown in SEQ ID NO:1 or SEQ ID NO:3, or a fragment of these nucleotide sequences.

When the polynucleotides of the invention are used for the recombinant production of RGS9 and evectin polypeptides of the present invention, the polynucleotide may include the coding sequence for the mature polypeptide, by itself, or the coding sequence for the mature polypeptide in reading frame with other coding sequences, such as those encoding a leader or secretory sequence, a pre-, or pro- or prepro-polypeptide sequence, or other fusion peptide portions. For example, a marker sequence which facilitates purification of the fused polypeptide can be encoded (see Gentz et al., 1989, incorporated herein by reference). The polynucleotide may also contain non-coding 5′ and 3′ sequences, such as transcribed, non-translated sequences, splicing and polyadenylation signals, ribosome binding sites and sequences that stabilize mRNA.

In addition to the RGS9 and evectin nucleotide sequences shown in SEQ ID NO:1 and SEQ ID NO:3, it will be appreciated by those skilled in the art that DNA sequence polymorphisms that lead to changes in the amino acid sequences of RGS9 or evectin polypeptides may exist within a population (e.g., the human population). Such genetic polymorphism in the RGS9 or evectin gene or polynucleotide may exist among individuals within a population due to natural allelic variation. As used herein, the terms “gene” and “recombinant gene” refer to polynucleotides comprising an open reading frame encoding a RGS9 or evectin polypeptide, preferably a human RGS9 and evectin polypeptide. Such natural allelic variations can typically result in 1-5% variance in the nucleotide sequence of the RGS9 or evectin polynucleotide. Any and all such nucleotide variations and resulting amino acid polymorphisms in a RGS9 or evectin polynucleotide that are the result of natural allelic variation are intended to be within the scope of the invention. Such allelic variation includes both active allelic variants as well as non-active or reduced activity allelic variants, the latter two types typically giving rise to a pathological disorder.

Moreover, nucleic acid molecules encoding RGS9 or evectin polypeptides from other species, and thus which have a nucleotide sequence which differs from the human sequence of SEQ ID NO:1 or SEQ ID NO:3, are intended to be within the scope of the invention. Polynucleotides corresponding to natural allelic variants and non-human orthologues of the human RGS9 and evectin cDNA of the invention can be isolated based on their homology to the human RGS9 and evectin polynucleotides disclosed herein using the human cDNA, or a fragment thereof, as a hybridization probe according to standard hybridization techniques under stringent hybridization conditions.

Thus, a polynucleotide encoding a polypeptide of the present invention, including homologs and orthologs from species other than human, may be obtained by a process which comprises the steps of screening an appropriate library under stringent hybridization conditions with a labeled probe having the sequence of SEQ ID NO:1 or SEQ ID NO:3, or a fragment thereof; and isolating full-length cDNA and genomic clones containing the polynucleotide sequence. Such hybridization techniques are well known to the skilled artisan. The skilled artisan will appreciate that, in many cases, an isolated cDNA sequence will be incomplete, in that the region coding for the polypeptide is cut short at the 5′ end of the cDNA. This is a consequence of reverse transcriptase, an enzyme with inherently low “processivity” (a measure of the ability of the enzyme to remain attached to the template during the polymerization reaction), failing to complete a DNA copy of the mRNA template during 1st strand cDNA synthesis.

Thus, in certain embodiments, the polynucleotide sequence information provided by the present invention allows for the preparation of relatively short DNA (or RNA) oligonucleotide sequences having the ability to specifically hybridize to gene sequences of the selected polynucleotides disclosed herein. The term “oligonucleotide” as used herein is defined as a molecule comprised of two or more deoxyribonucleotides or ribonucleotides, usually more than three (3), and typically more than ten (10) and up to one hundred (100) or more (although preferably between twenty and thirty). The exact size will depend on many factors, which in turn depends on the ultimate function or use of the oligonucleotide. Thus, in particular embodiments of the invention, nucleic acid probes of an appropriate length are prepared based on a consideration of a selected nucleotide sequence, e.g., a sequence such as that shown in SEQ ID NO:1 or SEQ ID NO:3. The ability of such nucleic acid probes to specifically hybridize to a polynucleotide encoding a RGS9 or evectin polypeptide lends them particular utility in a variety of embodiments. Most importantly, the probes can be used in a variety of assays for detecting the presence of complementary sequences in a given sample.

In certain embodiments, it is advantageous to use oligonucleotide primers. These primers may be generated in any manner, including chemical synthesis, DNA replication, reverse transcription, or a combination thereof. The sequence of such primers is designed using a polynucleotide of the present invention for use in detecting, amplifying or mutating a defined segment of a gene or polynucleotide that encodes a RGS9 or evectin polypeptide from mammalian cells using polymerase chain reaction (PCR) technology.

In certain embodiments, it is advantageous to employ a polynucleotide of the present invention in combination with an appropriate label for detecting hybrid formation. A wide variety of appropriate labels are known in the art, including radioactive, enzymatic or other ligands, such as avidin/biotin, which are capable of giving a detectable signal.

Polynucleotides which are identical or sufficiently identical to a nucleotide sequence contained in SEQ ID NO:1 or SEQ ID NO:3, or a fragment thereof, may be used as hybridization probes for cDNA and genomnic DNA or as primers for a nucleic acid amplification (PCR) reaction, to isolate full-length cDNAs and genomic clones encoding polypeptides of the present invention and to isolate cDNA and genomic clones of other genes (including genes encoding homologs and orthologs from species other than human) that have a high sequence similarity to SEQ ID NO:1 or SEQ ID NO:3, or a fragment thereof. Typically these nucleotide sequences are from at least about 70% identical to at least about 95% identical to that of the reference polynucleotide sequence. The probes or primers will generally comprise at least 15 nucleotides, preferably, at least 30 nucleotides and may have at least 50 nucleotides. Particularly preferred probes will have between 30 and 50 nucleotides.

There are several methods available and well known to those skilled in the art to obtain full-length cDNAs, or extend short cDNAs, for example those based on the method of Rapid Amplification of cDNA ends (RACE) (see, Frohman et al., 1988). Recent modifications of the technique, exemplified by the Marathon™ technology (Clontech Laboratories Inc.) for example, have significantly simplified the search for longer cDNAs. In the Marathon™ technology, cDNAs have been prepared from mRNA extracted from a chosen tissue and an “adaptor” sequence ligated onto each end. Nucleic acid amplification (PCR) is then carried out to amplify the “missing” 5′ end of the cDNA using a combination of gene specific and adaptor specific oligonucleotide primers. The PCR reaction is then repeated using “nested” primers, that is, primers designed to anneal within the amplified product (typically an adaptor specific primer that anneals further 3′ in the adaptor sequence and a gene specific primer that anneals further 5′ in the known gene sequence). The products of this reaction can then be analyzed by DNA sequencing and a full-length cDNA constructed either by joining the product directly to the existing cDNA to give a complete sequence, or carrying out a separate full-length PCR using the new sequence information for the design of the 5′ primer.

To provide certain of the advantages in accordance with the present invention, a preferred nucleic acid sequence employed for hybridization studies or assays includes probe molecules that are complementary to at least a 10 to 70 or more long nucleotide stretch of a polynucleotide that encodes a RGS9 or evectin polypeptide, such as that shown in SEQ ID NO:2 or SEQ ID NO:4. A size of at least 10 nucleotides in length helps to ensure that the fragment will be of sufficient length to form a duplex molecule that is both stable and selective. Molecules having complementary sequences over stretches greater than 10 bases in length are generally preferred, though, in order to increase stability and selectivity of the hybrid, and thereby improve the quality and degree of specific hybrid molecules obtained. One will generally prefer to design nucleic acid molecules having gene-complementary stretches of 25 to 40 nucleotides, 55 to 70 nucleotides, or even longer where desired. Such fragments can be readily prepared by, for example, directly synthesizing the fragment by chemical means, by application of nucleic acid reproduction technology, such as the PCR technology of (U.S. Pat. No. 4,683,202, incorporated by reference herein in its entirety) or by excising selected DNA fragments from recombinant plasmids containing appropriate inserts and suitable restriction enzyme sites.

In another aspect, the present invention contemplates an isolated and purified polynucleotide comprising a base sequence that is identical or complementary to a segment of at least 10 contiguous bases of SEQ ID NO:1 or SEQ ID NO:3, wherein the polynucleotide hybridizes to a polynucleotide that encodes a RGS9 or evectin polypeptide. Preferably, the isolated and purified polynucleotide comprises a base sequence that is identical or complementary to a segment of at least 25 to 70 contiguous bases of SEQ ID NO: 1 or SEQ ID NO:3. For example, the polynucleotide of the invention can comprise a segment of bases identical or complementary to 40 or 55 contiguous bases of the disclosed nucleotide sequences.

Accordingly, a polynucleotide probe molecule of the invention can be used for its ability to selectively form duplex molecules with complementary stretches of the gene. Depending on the application envisioned, one will desire to employ varying conditions of hybridization to achieve varying degree of selectivity of the probe toward the target sequence. For applications requiring a high degree of selectivity, one will typically desire to employ relatively stringent conditions to form the hybrids (see Table 1 below).

Of course, for some applications, for example, where one desires to prepare mutants employing a mutant primer strand hybridized to an underlying template or where one seeks to isolate a RGS9 or evectin polynucleotide coding sequence from other cells, functional equivalents, or the like, less stringent hybridization conditions are typically needed to allow formation of the heteroduplex. Cross-hybridizing species can thereby be readily identified as positively hybridizing signals with respect to control hybridizations. In any case, it is generally appreciated that conditions can be rendered more stringent by the addition of increasing amounts of formamide, which serves to destabilize the hybrid duplex in the same manner as increased temperature. Thus, hybridization conditions can be readily manipulated, and thus will generally be a method of choice depending on the desired results.

The present invention also includes polynucleotides capable of hybridizing under reduced stringency conditions, more preferably stringent conditions, and most preferably highly stringent conditions, to polynucleotides described herein. Examples of stringency conditions are shown in Table 1 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 1
Stringency Conditions

| | Polynucleo- | Hybrid | Hybridization | Wash |
|---|---|---|---|---|
| Stringency | tide | Length | Temperature and | Temperature |
| Condition | Hybrid | (bp)^{I} | Buffer^{H} | and BufferH |
| A | DNA:DNA | >50 | 65° C.; 1xSSC -or- | 65° C.; |
| | | | 42° C.; 1xSSC, 50% | 0.3xSSC |
| | | | formamide | |
| B | DNA:DNA | <50 | T_{B}; 1xSSC | T_{B}; 1xSSC |
| C | DNA:RNA | >50 | 67° C.; 1xSSC -or- | 67° C.; |
| | | | 45° C.; 1xSSC, 50% | 0.3xSSC |
| | | | formamide | |
| D | DNA:RNA | <50 | T_{D}; 1xSSC | T_{D}; 1xSSC |
| E | RNA:RNA | >50 | 70° C.; 1xSSC -or- | 70° C.; |
| | | | 50° C.; 1xSSC, 50% | 0.3xSSC |
| | | | formamide | |
| F | RNA:RNA | <50 | T_{F}; 1xSSC | T_{F}; 1xSSC |
| G | DNA:DNA | >50 | 65° C.; 4xSSC -or- | 65° C.; 1xSSC |
| | | | 42° C.; 4xSSC, 50% | |
| | | | formamide | |
| H | DNA:DNA | <50 | T_{H}; 4xSSC | T_{H}; 4xSSC |
| I | DNA:RNA | >50 | 67° C.; 4xSSC -or- | 67° C.; 1xSSC |
| | | | 45° C.; 4xSSC, 50% | |
| | | | formamide | |
| J | DNA:RNA | <50 | T_{J}; 4xSSC | T_{J}; 4xSSC |
| K | RNA:RNA | >50 | 70° C.; 4xSSC -or- | 67° C.; 1xSSC |
| | | | 50° C.; 4xSSC, 50% | |
| | | | formamide | |
| L | RNA:RNA | <50 | T_{L}; 2xSSC | T_{L}; 2xSSC |
| M | DNA:DNA | >50 | 50° C.; 4xSSC -or- | 50° C.; 2xSSC |
| | | | 40° C.; 6xSSC, 50% | |
| | | | formamide | |
| N | DNA:DNA | <50 | T_{N}; 6xSSC | T_{N}; 6xSSC |
| O | DNA:RNA | >50 | 55° C.; 4xSSC -or- | 55° C.; 2xSSC |
| | | | 42° C.; 6xSSC, 50% | |
| | | | formamide | |
| P | DNA:RNA | <50 | T_{P}; 6xSSC | T_{P}; 6xSSC |
| Q | RNA:RNA | >50 | 60° C.; 4xSSC -or- | 60° C.; 2xSSC |
| | | | 45° C.; 6xSSC, 50% | |
| | | | formamide | |
| R | RNA:RNA | <50 | T_{R}; 4xSSC | T_{R}; 4xSSC |
| (bp)^{I}: The hybrid length is that anticipated for the hybridized region(s) of the hybridizing polynucleotides. When hybridizing a polynucleotide to a target polynucleotide of unknown sequence, the hybrid length is assumed to be that of the | | | | |
| #hybridizing polynucleotide. When polynucleotides of known sequence are hybridized, the hybrid length can be determined by aligning the sequences of the polynucleotides and identifying the region or regions of optimal sequence complementarity. | | | | |
| Buffer^{H}: SSPE (1xSSPE is 0.15 M NaCl, 10 mM NaH₂PO₄, and 1.25 mM EDTA, pH 7.4) can be substituted for SSC (1xSSC is 0.15 M NaCl and 15 mM sodium citrate) in the hybridization and wash buffers; washes are performed for 15 minutes after hybridization is complete. | | | | |
| T_{B} through T_{R}: The hybridization temperature for hybrids anticipated to be less than 50 base pairs in length should be 5-10° C. less than the melting temperature (Tₘ) of the hybrid, where Tₘ is determined according to the following equations. For | | | | |
| #hybrids less than 18 base pairs in length, Tₘ(° C.) = 2(# of A + T bases) + 4(# of G + C bases). For hybrids between 18 and 49 base pairs in length, Tₘ(° C.) = 81.5 + 16.6(log₁₀[Na⁺]) + 0.41(% G + C) −(600/N), | | | | |
| #where N is the number of bases in the hybrid, and [Na⁺] is the concentration of sodium ions in the hybridization buffer ([Na⁺] for 1xSSC = 0.165 M). | | | | |

Additional examples of stringency conditions for polynucleotide hybridization are provided in Sambrook et al., 1989, Molecular Cloning: A Laboratory Manual, Cold Spring Harbor Laboratory Press, Cold Spring Harbor, N.Y., chapters 9 and 11, and Ausubel et al., 1995, Current Protocols in Molecular Biology, eds., John Wiley & Sons, Inc., sections 2.10 and 6.3-6.4, incorporated herein by reference.

In addition to the nucleic acid molecules encoding RGS9 and evectin polypeptides described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire RGS9 or evectin coding strand, or to only a fragment thereof. In one embodiment, an antisense nucleic acid molecule is antisense to a “coding region” of the coding strand of a nucleotide sequence encoding a RGS9 or evectin polypeptide.

The term “coding region” refers to the region of the nucleotide sequence comprising codons which are translated into amino acid residues, e.g., the entire coding region of SEQ ID NO:1 or SEQ ID NO:3. In another embodiment, the antisense nucleic acid molecule is antisense to a “noncoding region” of the coding strand of a nucleotide sequence encoding a RGS9 or evectin polypeptide. The term “noncoding region” refers to 5′ and 3′ sequences which flank the coding region that are not translated into amino acids (i.e., also referred to as 5′ and 3′ untranslated regions).

Given the coding strand sequence encoding the RGS9 or evectin polypeptide disclosed herein (e.g., SEQ ID NO:1 or SEQ ID NO:3), antisense nucleic acids of the invention can be designed according to the rules of Watson and Crick base pairing. The antisense nucleic acid molecule can be complementary to the entire coding region of RGS9 or evectin mRNA, but more preferably is an oligonucleotide which is antisense to only a fragment of the coding or noncoding region of RGS9 or evectin mRNA. For example, the antisense oligonucleotide can be complementary to the region surrounding the translation start site of RGS9 or evectin mRNA.

An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, I-methylguanine, I-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. In addition, backbone modifications such as peptide nucleic acids (PNAs) are contemplated for use in the invention (see U.S. Pat. No. 6,201,103).

Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a RGS9 or evectin polypeptide to thereby inhibit expression of the polypeptide, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of an antisense nucleic acid molecule of the invention includes direct injection at a tissue site. Alternatively, an antisense nucleic acid molecule can be modified to target selected cells and then administered systemically. For example, for systemic administration, an antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using the vectors described herein.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual γ-units, the strands run parallel to each other (Gaultier et al., 1987). The antisense nucleic acid molecule can also comprise a 2′-o-methylribonucleotide (Inoue et al., 1987(a)) or a chimeric RNA-DNA analogue (Inoue et al., 1987(b)).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach, 1988)) can be used to catalytically cleave RGS9 or evectin mRNA transcripts to thereby inhibit translation of RGS9 or evectin mRNA. A ribozyme having specificity for a RGS9 or evectin-encoding nucleic acid can be designed based upon the nucleotide sequence of a RGS9 or evectin cDNA disclosed herein (i.e., SEQ ID NO:1 or SEQ ID NO:3). For example, a derivative of a Tetrahymena L-19 IVS RNA can be constructed in which the nucleotide sequence of the active site is complementary to the nucleotide sequence to be cleaved in a RGS9 or evectin-encoding mRNA. See, e.g., Cech et al. U.S. Pat. No. 4,987,071 and Cech et al. U.S. Pat. No. 5,116,742 both of which are incorporated by reference herein in its entirety. Alternatively, RGS9 or evectin mRNA can be used to select a catalytic RNA having a specific ribonuclease activity from a pool of RNA molecules. See, e.g., Bartel and Szostak, 1993).

Alternatively RGS9 or evectin gene expression can be inhibited by targeting nucleotide sequences complementary to the regulatory region of the RGS9 or evectin gene (e.g. the RGS9 or evectin gene promoter and/or enhancers) to form triple helical structures that prevent transcription of the RGS9 or evectin gene in target cells. See generally, Helene, 1991; Helene et al., 1992; and Maher, 1992).

RGS9 or evectin gene expression can also be inhibited using RNA interference (RNAi). This is a technique for post-transcriptional gene silencing (PTGS), in which target gene activity is specifically abolished with cognate double-stranded RNA (dsRNA). RNAi resembles in many aspects PTGS in plants and has been detected in many invertebrates including trypanosome, hydra, planaria, nematode and fruit fly (Drosophila melangnoster). It may be involved in the modulation of transposable element mobilization and antiviral state formation. RNAi in mammalian systems is disclosed in International Application No. WO 00/63364 which is incorporated by reference herein in its entirety. Basically, dsRNA of at least about 600 nucleotides, homologous to the target (RGS9 or evectin) is introduced into the cell and a sequence specific reduction in gene activity is observed.

B. RGS9 and Evectin Polypeptides

In particular embodiments, the present invention provides isolated and purified RGS9 or evectin polypeptides and fragments thereof. Preferably, a RGS9 or evectin polypeptide of the invention is a recombinant polypeptide. Typically, a RGS9 or evectin is produced by recombinant expression in a non-human cell. In certain embodiments, a RGS9 polypeptide of the present invention comprises the amino acid sequence of SEQ ID NO:2, a variant thereof or a fragment thereof. In certain other embodiments, an isolated RGS9 polypeptide is a fragment comprising an evectin polypeptide binding domain, wherein the RGS9 polypeptide fragment comprises the amino acid sequence from amino acid 461 through amino acid 602 of SEQ ID NO:2. In another embodiment, an evectin polypeptide of the present invention comprises the amino acid sequence of SEQ ID NO:4, a variant thereof or a fragment thereof. In another embodiment, an isolated evectin polypeptide is a fragment comprising a RGS9 polypeptide binding domain, wherein the evectin polypeptide fragment comprises the amino acid sequence from amino acid 79 through amino acid 136 of SEQ ID NO:4.

A RGS9 or evectin polypeptide according to the present invention encompasses a polypeptide that comprises: 1) the amino acid sequence shown in SEQ ID NO:2 or SEQ ID NO:4; 2) functional and non-functional naturally occurring allelic variants of human RGS9 or evectin polypeptides; 3) recombinantly produced variants of human RGS9 or evectin polypeptides; and 4) RGS9 or evectin polypeptides isolated from organisms other than humans (orthologues of human RGS9 or evectin polypeptides.)

An allelic variant of human RGS9 or evectin polypeptides according to the present invention encompasses 1) a polypeptide isolated from human cells or tissues; 2) a polypeptide encoded by the same genetic locus as that encoding the human RGS9 or evectin polypeptide; and 3) a polypeptide that contains substantially homology to a human RGS9 or evectin.

Allelic variants of human RGS9 or evectin include both functional and non-functional RGS9 or evectin polypeptides. Functional allelic variants are naturally occurring amino acid sequence variants of the human RGS9 or evectin polypeptide that maintain the ability to bind a RGS9 or evectin ligand and transduce a signal within a cell. Functional allelic variants will typically contain only a conservative substitution of one or more amino acids of SEQ ID NO:2 or SEQ ID NO:4 or a substitution, deletion or insertion of non-critical residues in non-critical regions of the polypeptide.

Non-functional allelic variants are naturally occurring amino acid sequence variants of human RGS9 or evectin polypeptides that do not have the ability to either bind ligand and/or transduce a signal within a cell. Non-functional allelic variants will typically contain a non-conservative substitution, a deletion, or insertion or premature truncation of the amino acid sequence of SEQ ID NO:2 or SEQ ID NO:4, or a substitution, insertion or deletion in critical residues or critical regions.

The present invention further provides non-human orthologues of human RGS9 or evectin polypeptides. Orthologues of human RGS9 or evectin polypeptide are polypeptides that are isolated from non-human organisms and possess the same ligand binding and signaling capabilities of the human RGS9 or evectin polypeptide. Orthologues of the human RGS9 or evectin polypeptide can readily be identified as comprising an amino acid sequence that is substantially homologous to SEQ ID NO:2 or SEQ ID NO:4.

Modifications and changes can be made in the structure of a polypeptide of the present invention and still obtain a molecule having RGS9 or evectin-like characteristics. For example, certain amino acids can be substituted for other amino acids in a sequence without appreciable loss of receptor activity. Because it is the interactive capacity and nature of a polypeptide that defines that polypeptide's biological functional activity, certain amino acid sequence substitutions can be made in a polypeptide sequence (or, of course, its underlying DNA coding sequence) and nevertheless obtain a polypeptide with like properties.

In making such changes, the hydropathic index of amino acids can be considered. The importance of the hydropathic amino acid index in conferring interactive biologic function on a polypeptide is generally understood in the art (Kyte & Doolittle, 1982). It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid residue determines the secondary and tertiary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within +/−2 is preferred, those which are within +/−1 are particularly preferred, and those within +/−0.5 are even more particularly preferred.

Substitution of like amino acids can also be made on the basis of hydrophilicity, particularly where the biological functional equivalent polypeptide or peptide thereby created is intended for use in immunological embodiments. U.S. Pat. No. 4,554,101, incorporated reference herein in its entirety, states that the greatest local average hydrophilicity of a polypeptide, as governed by the hydrophilicity of its adjacent amino acids, correlates with its immunogenicity and antigenicity, i.e. with a biological property of the polypeptide.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those which are within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine (see Table 2, below). The present invention thus contemplates functional or biological equivalents of a RGS9 or evectin polypeptide as set forth above.

TABLE 2

| Original | Exemplary Residue |
|---|---|
| Residue | Substitution |
| Ala | Gly; Ser |
| Arg | Lys |
| Asn | Gln; His |
| Asp | Glu |
| Cys | Ser |
| Gln | Asn |
| Glu | Asp |
| Gly | Ala |
| His | Asn; Gln |
| Ile | Leu; Val |
| Leu | Ile; Val |
| Lys | Arg |
| Met | Leu; Tyr |
| Ser | Thr |
| Thr | Ser |
| Trp | Tyr |
| Tyr | Trp; Phe |
| Val | Ile; Leu |

Biological or functional equivalents of a polypeptide can also be prepared using site-specific mutagenesis. Site-specific mutagenesis is a technique useful in the preparation of second generation polypeptides, or biologically functional equivalent polypeptides or peptides, derived from the sequences thereof, through specific mutagenesis of the underlying DNA. As noted above, such changes can be desirable where amino acid substitutions are desirable. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

In general, the technique of site-specific mutagenesis is well known in the art. As will be appreciated, the technique typically employs a phage vector which can exist in both a single stranded and double stranded form. Typically, site-directed mutagenesis in accordance herewith is performed by first obtaining a single-stranded vector which includes within its sequence a DNA sequence which encodes all or a portion of the RGS9 or evectin polypeptide sequence selected. An oligonucleotide primer bearing the desired mutated sequence is prepared (e.g., synthetically). This primer is then annealed to the single-stranded vector, and extended by the use of enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells such as E. coli cells and clones are selected which include recombinant vectors bearing the mutation. Commercially available kits come with all the reagents necessary, except the oligonucleotide primers.

The RGS9 or evectin polypeptide is a RGS9 or evectin that participates in signaling pathways within cells. As used herein, a signaling pathway refers to the modulation (e.g., stimulated or inhibited) of a cellular function/activity upon the binding of a ligand to the RGS9 or evectin (RGS9 or evectin polypeptide). Examples of such functions include mobilization of intracellular molecules that participate in a signal transduction pathway, e.g., phosphatidylinositol 4,5-bisphosphate (PIP₂), inositol 1,4,5-triphosphate ON or adenylate cyclase; polarization of the plasma membrane; production or secretion of molecules; alteration in the structure of a cellular component; cell proliferation, e.g., synthesis of DNA; cell migration; cell differentiation; and cell survival. As the RGS9 polypeptide identified is expressed substantially in the brain, examples of cells participating in a RGS9 signaling pathway are contemplated in the present invention and include neural cells, e.g. peripheral nervous system and central nervous system cells such as brain cells, e.g., limbic system cells, hypothalamus cells, hippocampus cells, substantia nigra cells, cortex cells, brain stem cells, neocortex cells, basal ganglion cells, caudate putamen cells, olfactory tubercle cells, and superior colliculi cells.

Depending on the type of cell, the response mediated by the RGS9 or evectin polypeptide/ligand binding may be different. For example, in some cells, binding of a ligand to a RGS9 or evectin polypeptide may stimulate an activity such as adhesion, migration, differentiation, etc. through phosphatidylinositol or cyclic AMP metabolism and turnover while in other cells, the binding of the ligand to the RGS9 or evectin polypeptide will produce a different result. Regardless of the cellular activity modulated by RGS9 or evectin, it is universal that the RGS9 or evectin polypeptide is a RGS9 or evectin and interacts directly or indirectly with a “G-polypeptide” to produce one or more secondary signals in a variety of intracellular signal transduction pathways, e.g., through phosphatidylinositol or cyclic AMP metabolism and turnover, in a cell. G-polypeptides represent a family of heterotrimeric polypeptides composed of α, β and γ subunits, which bind guanine nucleotides. These polypeptides are usually linked to cell surface receptors, e.g., receptors containing seven transmembrane domains, such as the ligand receptors. Following ligand binding to the receptor, a conformational change is transmitted to the G-polypeptide, which causes the α-subunit to exchange a bound GDP molecule for a GTP molecule and to dissociate from the N-subunits. The GTP-bound form of the α-subunit typically functions as an effector-modulating moiety, leading to the production of second messengers, such as cyclic AMP (e.g., by activation of adenylate cyclase), diacylglycerol or inositol phosphates. Greater than 20 different types of α-subunits are known in man, which associate with a smaller pool of β and γ subunits.

As used herein, “phosphatidylinositol turnover and metabolism” refers to the molecules involved in the turnover and metabolism of phosphatidylinositol 4,5-bisphosphate (PIP₂) as well as to the activities of these molecules. PIP₂ is a phospholipid found in the cytosolic leaflet of the plasma membrane. Binding of a ligand to the RGS9 or evectin activates, in some cells, the plasma-membrane enzyme phospholipase C that in turn can hydrolyze PIP₂ to produce 1,2-diacylglycerol (DAG) and inositol 1,4,5-triphosphate IP₃). Once formed, IP₃ can diffuse to the endoplasmic reticulum surface where it can bind an IP₃ receptor, e.g., a calcium channel polypeptide containing an IP₃ binding site. IP₃ binding can induce opening of the channel, allowing calcium ions to be released into the cytoplasm. IP₃ can also be phosphorylated by a specific kinase to form inositol 1,3,4,5-tetraphosphate, a molecule which can cause calcium entry into the cytoplasm from the extracellular medium. IP₃ and IN can subsequently be hydrolyzed very rapidly to the inactive products inositol 1,4-biphosphate ON and inositol 1,3,4-triphosphate, respectively. These inactive products can be recycled by the cell to synthesize PIP₂. The other second messenger produced by the hydrolysis of PIP₂, namely 1,2-diacylglycerol (DAG), remains in the cell membrane where it can serve to activate the enzyme polypeptide kinase C. Polypeptide kinase C is usually found soluble in the cytoplasm of the cell, but upon an increase in the intracellular calcium concentration, this enzyme can move to the plasma membrane where it can be activated by DAG. The activation of polypeptide kinase C in different cells results in various cellular responses such as the phosphorylation of glycogen synthase, or the phosphorylation of various transcription factors, e.g., NF-kB. The language “phosphatidylinositol activity,” as used herein, refers to an activity of PIP₂ or one of its metabolites.

Another signaling pathway in which the RGS9 or evectin polypeptide may participate is the cAMP turnover pathway. As used herein, “cyclic AMP turnover and metabolism” refers to the molecules involved in the turnover and metabolism of cyclic AMP (cAMP) as well as to the activities of these molecules. Cyclic AMP is a second messenger produced in response to ligand induced stimulation of certain G-polypeptide coupled receptors. In the ligand signaling pathway, binding of ligand to a ligand receptor can lead to the activation of the enzyme adenylyl cyclase, which catalyzes the synthesis of cAMP. The newly synthesized cAMP can in turn activate a cAMP-dependent polypeptide kinase. This activated kinase can phosphorylate a voltage-gated potassium channel polypeptide, or an associated polypeptide, and lead to the inability of the potassium channel to open during an action potential. The inability of the potassium channel to open results in a decrease in the outward flow of potassium, which normally repolarizes the membrane of a neuron, leading to prolonged membrane depolarization.

A RGS9 or evectin polypeptide of the present invention is understood to be any RGS9 or evectin polypeptide comprising substantial sequence similarity, structural similarity and/or functional similarity to a RGS9 or evectin polypeptide comprising the amino acid sequence selected from the group consisting of SEQ ID NO:2 or SEQ ID NO:4. In addition, a RGS9 or evectin polypeptide of the invention is not limited to a particular source. Thus, the invention provides for the general detection and isolation of the genus of RGS9 or evectin polypeptides from a variety of sources. Where there is a difference between species, identification of those differences is well within the skill of an artisan. Thus, the present invention contemplates a RGS9 or evectin polypeptide from any mammal, wherein the preferred mammal is a human.

It is contemplated in the present invention, that a RGS9 or evectin may advantageously be cleaved into fragments for use in further structural or functional analysis, or in the generation of reagents such as RGS9 or evectin-related polypeptides and RGS9 or evectin-specific antibodies. This can be accomplished by treating purified or unpurified RGS9 or evectin with a peptidase such as endopolypeptidease glu-C (Boehringer, Indianapolis, Ind.). Treatment with CNBr is another method by which RGS9 or evectin fragments may be produced from natural RGS9 or evectin. Recombinant techniques also can be used to produce specific fragments of RGS9 or evectin

In addition, it also is contemplated that compounds sterically similar to a RGS9 or evectin may be formulated to mimic the key portions of the peptide structure, called peptidomimetics. Mimetics are peptide-containing molecules which mimic elements of polypeptide secondary structure. See, for example, Johnson et al. (1993). The underlying rationale behind the use of peptide mimetics is that the peptide backbone of polypeptides exists chiefly to orient amino acid side chains in such a way as to facilitate molecular interactions, such as those of receptor and ligand.

Successful applications of the peptide mimetic concept have thus far focused on mimetics of β-turns within polypeptides. Likely β-turn structures within RGS9 or evectin can be predicted by computer-based algorithms as discussed above. Once the component amino acids of the turn are determined, mimetics can be constructed to achieve a similar spatial orientation of the essential elements of the amino acid side chains, as discussed in Johnson et al. (1993).

“Fusion polypeptide” refers to a polypeptide encoded by two, often unrelated, fused genes or fragments thereof For example, fusion polypeptides comprising various portions of constant region of immunoglobulin molecules together with another human polypeptide or part thereof have been described. In many cases, employing an immunoglobulin Fc region as a part of a fusion polypeptide is advantageous for use in therapy and diagnosis resulting in, for example, improved pharmacokinetic properties (see, e.g., International Application No. EP-A 0232 2621). On the other hand, for some uses it would be desirable to be able to delete the Fc part after the fusion polypeptide has been expressed, detected and purified.

C. Vectors, Host Cells and Recombinant RGS9 and Evectin Polypeptides

In an alternate embodiment, the present invention provides expression vectors comprising polynucleotides that encode RGS9 polypeptide fragments, evectin polypeptide fragments, or RGS9-evectin dimers or fragments thereof. Preferably, the expression vectors of the present invention comprise polynucleotides that encode polypeptides comprising the amino acid residue sequence of SEQ ID NO:2 or SEQ ID NO:4. More preferably, the expression vectors of the present invention comprise polynucleotides comprising the nucleotide base sequence of SEQ ID NO:1 or SEQ ID:3. Even more preferably, the expression vectors of the invention comprise polynucleotides operatively linked to an enhancer-promoter. In certain embodiments, the expression vectors of the invention comprise polynucleotides operatively linked to a prokaryotic promoter. Alternatively, the expression vectors of the present invention comprise polynucleotides operatively linked to an enhancer-promoter that is a eukaryotic promoter, and the expression vectors further comprise a polyadenylation signal that is positioned 3′ of the carboxy-terminal amino acid and within a transcriptional unit of the encoded polypeptide.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

Typical fusion expression vectors include pGEX (Pharmacia Biotech Inc; Smith and Johnson,1988), pMAL (New England Biolabs, Beverly; Mass.) and pRIT5 (Pharmacia, Piscataway, N.J.) which fuse glutathione S-transferase (GST), maltose E binding protein, or protein A, respectively, to the target recombinant protein.

In one embodiment, the coding sequence of the RGS9 or evectin gene is cloned into a pGEX expression vector to create a vector encoding a fusion protein comprising, from the N-terminus to the C-terminus, GST-thrombin cleavage site-RGS9 or -evectin polypeptide. The fusion protein can be purified by affinity chromatography using glutathione-agarose resin. Recombinant RGS9 or evectin polypeptide unfused to GST can be recovered by cleavage of the fusion protein with thrombin.

Examples of suitable inducible non-fusion E. coli expression vectors include pTrc (Amann et al., 1988) and pET I I d (Studier et al., 1990). Target gene expression from the pTrc vector relies on host RNA polymerase transcription from a hybrid trp-lac fusion promoter. Target gene expression from the pET I I d vector relies on transcription from a T7 gn1 0-lac fusion promoter mediated by a coexpressed viral RNA polymerase J7 gnl. This viral polymerase is supplied by host strains BL21 (DE3) or HMS I 74(DE3) from a resident prophage harboring a T7 gnI gene under the transcriptional control of the lacUV 5 promoter.

One strategy to maximize recombinant protein expression in E. coli is to express the protein in a host bacteria with an impaired capacity to proteolytically cleave the recombinant protein. Another strategy is to alter the nucleic acid sequence of the nucleic acid to be inserted into an expression vector so that the individual codons for each amino acid are those preferentially utilized in E. coli. Such alteration of nucleic acid sequences of the invention can be carried out by standard DNA mutagenesis or synthesis techniques.

In another embodiment, the RGS9 or evectin polynucleotide expression vector is a yeast expression vector. Examples of vectors for expression in yeast S. cerivisae include pYepSec I (Baldari, et al., 1987), pMFa (Kujan and Herskowitz, 1982), pJRY88 (Schultz et al., 1987), and pYES2 (Invitrogen Corporation, San Diego, Calif.).

Alternatively, a RGS9 or evectin polynucleotide can be expressed in insect cells using, for example, baculovirus expression vectors. Baculovirus vectors available for expression of proteins in cultured insect cells (e.g., Sf9 cells) include the pAc series (Smith et al., 1983) and the pVL series (Lucklow and Summers, 1989).

In yet another embodiment, a polynucleotide of the invention is expressed in mammalian cells using a mammalian expression vector. Examples of mammalian expression vectors include pCDM8 (Seed, 1987), pCDNA3-1 (Invitrogen) and pMT2PC (Kaufman et al., 1987). When used in mammalian cells, the expression vector's control functions are often provided by viral regulatory elements.

For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., “Molecular Cloning: A Laboratory Manual” 2nd, ed, Cold Spring Harbor Laboratory, Cold Spring Harbor Laboratory Press, Cold Spring Harbor, N.Y., 1989, incorporated by reference herein in its entirety.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., 1987), lymphoid-specific promoters (Calame and Eaton, 1988), in particular promoters of T cell receptors (Winoto and Baltimore, 1989) and immunoglobulins (Banerji et al., 1983, Queen and Baltimore, 1983), neuron-specific promoters (e.g., the neurofilament promoter; Byrne and Ruddle, 1989), pancreas-specific promoters (Edlund et al., 1985), and mammary gland-specific promoters (e.g., milk whey promoter; U.S. Pat. No. 4,873,316 and International Application No. EP 264,166). Developmentally-regulated promoters are also encompassed, for example the murine hox promoters (Kessel and Gruss, 1990) and the α-fetoprotein promoter (Campes and Tilghman, 1989).

The invention further provides a recombinant expression vector comprising a DNA molecule encoding a RGS9 or evectin polypeptide cloned into the expression vector in an antisense orientation. That is, the DNA molecule is operatively linked to a regulatory sequence in a manner which allows for expression (by transcription of the DNA molecule) of an RNA molecule which is antisense to RGS9 or evectin mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. For example, RGS9 or evectin polypeptide can be expressed in bacterial cells such as E coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation, infection or transfection techniques. As used herein, the terms “transformation” and “transfection” are intended to refer to a variety of art-recognized techniques for introducing foreign nucleic acid (e.g. DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (“Molecular Cloning: A Laboratory Manual” 2nd. Ed. Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding the RGS9 or evectin polypeptide or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) RGS9 or evectin polypeptides. Accordingly, the invention further provides methods for producing RGS9 or evectin polypeptides using the host cells of the invention. In one embodiment, the method comprises culturing the host cell of invention (into which a recombinant expression vector encoding a RGS9 or evectin polypeptide has been introduced) in a suitable medium until the RGS9 or evectin polypeptide is produced. In another embodiment, the method further comprises isolating the RGS9 or evectin polypeptide from the medium or the host cell.

A promoter is a region of a DNA molecule typically within about 100 nucleotide pairs in front of (upstream of) the point at which transcription begins (i.e., a transcription start site). That region typically contains several types of DNA sequence elements that are located in similar relative positions in different genes. As used herein, the term “promoter” includes what is referred to in the art as an upstream promoter region, a promoter region or a promoter of a generalized eukaryotic RNA Polymerase II transcription unit.

Another type of discrete transcription regulatory sequence element is an enhancer. An enhancer provides specificity of time, location and expression level for a particular encoding region (e.g., gene). A major function of an enhancer is to increase the level of transcription of a coding sequence in a cell that contains one or more transcription factors that bind to that enhancer. Unlike a promoter, an enhancer can function when located at variable distances from transcription start sites so long as a promoter is present.

As used herein, the phrase “enhancer-promoter” means a composite unit that contains both enhancer and promoter elements. An enhancer-promoter is operatively linked to a coding sequence that encodes at least one gene product. As used herein, the phrase “operatively linked” means that an enhancer-promoter is connected to a coding sequence in such a way that the transcription of that coding sequence is controlled and regulated by that enhancer-promoter. Means for operatively linking an enhancer-promoter to a coding sequence are well known in the art. As is also well known in the art, the precise orientation and location relative to a coding sequence whose transcription is controlled, is dependent inter alia upon the specific nature of the enhancer-promoter. Thus, a TATA box minimal promoter is typically located from about 25 to about 30 base pairs upstream of a transcription initiation site and an upstream promoter element is typically located from about 100 to about 200 base pairs upstream of a transcription initiation site. In contrast, an enhancer can be located downstream from the initiation site and can be at a considerable distance from that site.

An enhancer-promoter used in a vector construct of the present invention can be any enhancer-promoter that drives expression in a cell to be transfected. By employing an enhancer-promoter with well-known properties, the level and pattern of gene product expression can be optimized.

A coding sequence of an expression vector is operatively linked to a transcription terminating region. RNA polymerase transcribes an encoding DNA sequence through a site where polyadenylation occurs. Typically, DNA sequences located a few hundred base pairs downstream of the polyadenylation site serve to terminate transcription. Those DNA sequences are referred to herein as transcription-termination regions. Those regions are required for efficient polyadenylation of transcribed messenger RNA (mRNA). Transcription-terminating regions are well known in the art. A preferred transcription-terminating region used in an adenovirus vector construct of the present invention comprises a polyadenylation signal of SV40 or the protamine gene.

An expression vector comprises a polynucleotide that encodes a RGS9 or evectin polypeptide. Such a polypeptide is meant to include a sequence of nucleotide bases encoding a RGS9 or evectin polypeptide sufficient in length to distinguish said segment from a polynucleotide segment encoding a non-RGS9 or -evectin polypeptide. A polypeptide of the invention can also encode biologically functional polypeptides or peptides which have variant amino acid sequences, such as with changes selected based on considerations such as the relative hydropathic score of the amino acids being exchanged. These variant sequences are those isolated from natural sources or induced in the sequences disclosed herein using a mutagenic procedure such as site-directed mutagenesis.

Preferably, the expression vectors of the present invention comprise polynucleotides that encode polypeptides comprising the amino acid residue sequence of SEQ ID NO:2 or SEQ ID NO:4. An expression vector can include a RGS9 or evectin polypeptide coding region itself of any of the RGS9 or evectin polypeptides noted above or it can contain coding regions bearing selected alterations or modifications in the basic coding region of such a RGS9 or evectin polypeptide. Alternatively, such vectors or fragments can code larger polypeptides or polypeptides which nevertheless include the basic coding region. In any event, it should be appreciated that due to codon redundancy as well as biological functional equivalence, this aspect of the invention is not limited to the particular DNA molecules corresponding to the polypeptide sequences noted above.

Exemplary vectors include the mammalian expression vectors of the pCMV family including pCMV6b and pCMV6c (Chiron Corp., Emeryville Calif.). In certain cases, and specifically in the case of these individual mammalian expression vectors, the resulting constructs can require co-transfection with a vector containing a selectable marker such as pSV2neo. Via co-transfection into a dihydrofolate reductase-deficient Chinese hamster ovary cell line, such as DG44, clones expressing RGS9 or evectin polypeptides by virtue of DNA incorporated into such expression vectors can be detected.

A DNA molecule, gene or polynucleotide of the present invention can be incorporated into a vector by a number of techniques which are well known in the art. For instance, the vector pUC18 has been demonstrated to be of particular value Likewise, the related vectors M13 mp18 and M13 mp19 can be used in certain embodiments of the invention, in particular, in performing dideoxy sequencing.

An expression vector of the present invention is useful both as a means for preparing quantities of the RGS9 or evectin polypeptide-encoding DNA itself, and as a means for preparing the encoded polypeptide and peptides. It is contemplated that where RGS9 or evectin polypeptides of the invention are made by recombinant means, one can employ either prokaryotic or eukaryotic expression vectors as shuttle systems. However, in that prokaryotic systems are usually incapable of correctly processing precursor polypeptides and, in particular, such systems are incapable of correctly processing membrane associated eukaryotic polypeptides, and since eukaryotic RGS9 or evectin polypeptides are anticipated using the teaching of the disclosed invention, one likely expresses such sequences in eukaryotic hosts. However, even where the DNA segment encodes a eukaryotic RGS9 or evectin polypeptide, it is contemplated that prokaryotic expression can have some additional applicability. Therefore, the invention can be used in combination with vectors which can shuttle between the eukaryotic and prokaryotic cells. Such a system is described herein which allows the use of bacterial host cells as well as eukaryotic host cells.

Where expression of recombinant RGS9 or evectin polypeptides is desired and a eukaryotic host is contemplated, it is most desirable to employ a vector such as a plasmid, that incorporates a eukaryotic origin of replication. Additionally, for the purposes of expression in eukaryotic systems, one desires to position the RGS9 or evectin encoding sequence adjacent to and under the control of an effective eukaryotic promoter such as promoters used in combination with Chinese hamster ovary cells. To bring a coding sequence under control of a promoter, whether it is eukaryotic or prokaryotic, what is generally needed is to position the 5′ end of the translation initiation side of the proper translational reading frame of the polypeptide between about 1 and about 50 nucleotides 3′ of or downstream with respect to the promoter chosen. Furthermore, where eukaryotic expression is anticipated, one would typically desire to incorporate into the transcriptional unit which includes the RGS9 or evectin polypeptide, an appropriate polyadenylation site.

The pCMV plasmids are a series of mammalian expression vectors of particular utility in the present invention. The vectors are designed for use in essentially all cultured cells and work extremely well in SV40-transformed simian COS cell lines. The pCMV1, 2, 3, and 5 vectors differ from each other in certain unique restriction sites in the polylinker region of each plasmid. The pCMV4 vector differs from these 4 plasmids in containing a translation enhancer in the sequence prior to the polylinker. While they are not directly derived from the pCMV1-5 series of vectors, the functionally similar pCMV6b and c vectors are available from the Chiron Corp. (Emeryville, Calif.) and are identical except for the orientation of the polylinker region which is reversed in one relative to the other.

The universal components of the pCMV plasmids are as follows. The vector backbone is pTZ18R (Pharmacia), and contains a bacteriophage f1 origin of replication for production of single stranded DNA and an ampicillin-resistance gene. The CMV region consists of nucleotides −760 to +3 of the powerful promoter-regulatory region of the human cytomegalovirus (Towne stain) major immediate early gene (Thomsen et al., 1984; Boshart et al., 1985). The human growth hormone fragment (hGH) contains transcription termination and poly-adenylation signals representing sequences 1533 to 2157 of this gene (Seeburg, 1982). There is an Alu middle repetitive DNA sequence in this fragment. Finally, the SV40 origin of replication and early region promoter-enhancer derived from the pcD-X plasmid (HindII to PstI fragment) described in (Okayama et al., 1983). The promoter in this fragment is oriented such that transcription proceeds away from the CMV/hGH expression cassette.

The pCMV plasmids are distinguishable from each other by differences in the polylinker region and by the presence or absence of the translation enhancer. The starting pCMV1 plasmid has been progressively modified to render an increasing number of unique restriction sites in the polylinker region. To create pCMV2, one of two EcoRI sites in pCMV1 were destroyed. To create pCMV3, pCMV1 was modified by deleting a short segment from the SV40 region (StuI to EcoRI), and in so doing made unique the PstI, SalI, and BamHI sites in the polylinker. To create pCMV4, a synthetic fragment of DNA corresponding to the 5′-untranslated region of a mRNA transcribed from the CMV promoter was added C. The sequence acts as a translational enhancer by decreasing the requirements for initiation factors in polypeptide synthesis (Jobling et al., 1987; Browning et al., 1988). To create pCMV5, a segment of DNA (HpaI to EcoRI) was deleted from the SV40 origin region of pCMV1 to render unique all sites in the starting polylinker.

The pCMV vectors have been successfully expressed in simian COS cells, mouse L cells, CHO cells, and HeLa cells. In several side by side comparisons they have yielded 5- to 10-fold higher expression levels in COS cells than SV40-based vectors. The pCMV vectors have been used to express the LDL receptor, nuclear factor 1, GS alpha polypeptide, polypeptide phosphatase, synaptophysin, synapsin, insulin receptor, influenza hemmagglutinin, androgen receptor, sterol 26-hydroxylase, steroid 17- and 21-hydroxylase, cytochrome P-450 oxidoreductase, beta-adrenergic receptor, folate receptor, cholesterol side chain cleavage enzyme, and a host of other cDNAs. It should be noted that the SV40 promoter in these plasmids can be used to express other genes such as dominant selectable markers. Finally, there is an ATG sequence in the polylinker between the HindIII and PstI sites in pCMU that can cause spurious translation initiation. This codon should be avoided if possible in expression plasmids. A paper describing the construction and use of the parenteral pCMV1 and pCMV4 vectors has been published (Anderson et al., 1989b).

In yet another embodiment, the present invention provides recombinant host cells transformed, infected or transfected with polynucleotides that encode RGS9 or evectin polypeptides, as well as transgenic cells derived from those transformed or transfected cells. Preferably, the recombinant host cells of the present invention are transfected with a polynucleotide of SEQ ID NO:1 or SEQ ID NO:3. Means of transforming or transfecting cells with exogenous polynucleotide such as DNA molecules are well known in the art and include techniques such as calcium-phosphate- or DEAE-dextran-mediated transfection, protoplast fusion, electroporation, liposome mediated transfection, direct microinjection and adenovirus infection (Sambrook, Fritsch and Maniatis, 1989).

The most widely used method is transfection mediated by either calcium phosphate or DEAE-dextran. Although the mechanism remains obscure, it is believed that the transfected DNA enters the cytoplasm of the cell by endocytosis and is transported to the nucleus. Depending on the cell type, up to 90% of a population of cultured cells can be transfected at any one time. Because of its high efficiency, transfection mediated by calcium phosphate or DEAE-dextran is the method of choice for experiments that require transient expression of the foreign DNA in large numbers of cells. Calcium phosphate-mediated transfection is also used to establish cell lines that integrate copies of the foreign DNA, which are usually arranged in head-to-tail tandem arrays into the host cell genome.

In the protoplast fusion method, protoplasts derived from bacteria carrying high numbers of copies of a plasmid of interest are mixed directly with cultured mammalian cells. After fusion of the cell membranes (usually with polyethylene glycol), the contents of the bacteria are delivered into the cytoplasm of the mammalian cells and the plasmid DNA is transported to the nucleus. Protoplast fusion is not as efficient as transfection for many of the cell lines that are commonly used for transient expression assays, but it is useful for cell lines in which endocytosis of DNA occurs inefficiently. Protoplast fusion frequently yields multiple copies of the plasmid DNA tandemly integrated into the host chromosome.

The application of brief, high-voltage electric pulses to a variety of mammalian and plant cells leads to the formation of nanometer-sized pores in the plasma membrane. DNA is taken directly into the cell cytoplasm either through these pores or as a consequence of the redistribution of membrane components that accompanies closure of the pores. Electroporation can be extremely efficient and can be used both for transient expression of cloned genes and for establishment of cell lines that carry integrated copies of the gene of interest. Electroporation, in contrast to calcium phosphate-mediated transfection and protoplast fusion, frequently gives rise to cell lines that carry one, or at most a few, integrated copies of the foreign DNA.

Liposome transfection involves encapsulation of DNA and RNA within liposomes, followed by fusion of the liposomes with the cell membrane. The mechanism of how DNA is delivered into the cell is unclear but transfection efficiencies can be as high as 90%.

Direct microinjection of a DNA molecule into nuclei has the advantage of not exposing DNA to cellular compartments such as low-pH endosomes. Microinjection is therefore used primarily as a method to establish lines of cells that carry integrated copies of the DNA of interest.

The use of adenovirus as a vector for cell transfection is well known in the art. Adenovirus vector-mediated cell transfection has been reported for various cells (Stratford-Perricaudet, et al. 1992).

A transfected cell can be prokaryotic or eukaryotic. Preferably, the host cells of the invention are eukaryotic host cells. The recombinant host cells of the invention may be COS-1 cells. Where it is of interest to produce a human polypeptide, cultured mammalian or human cells are of particular interest.

In another aspect, the recombinant host cells of the present invention are prokaryotic host cells. Preferably, the recombinant host cells of the invention are bacterial cells of the DH5 a strain of Escherichia coli. In general, prokaryotes are preferred for the initial cloning of DNA sequences and constructing the vectors useful in the invention. For example, E. coli K12 strains can be particularly useful. Other microbial strains which can be used include E. coli B, and E. coli_{X}1976 (ATCC No. 31537). These examples are, of course, intended to be illustrative rather than limiting.

Prokaryotes can also be used for expression. The aforementioned strains, as well as E. coli W3110 (ATCC No. 273325), bacilli such as Bacillus subtilis, or other enterobacteriaceae such as Salmonella typhimurium or Serratia marcesans, and various Pseudomonas species can be used.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli can be transformed using pBR322, a plasmid derived from an E. coli species (Bolivar, et al. 1977). pBR322 contains genes for ampicillin and tetracycline resistance and thus provides easy means for identifying transformed cells. The pBR plasmid, or other microbial plasmid or phage must also contain, or be modified to contain, promoters which can be used by the microbial organism for expression of its own polypeptides.

Those promoters most commonly used in recombinant DNA construction include the β-lactamase (penicillinase) and lactose promoter systems (Chang, et al. 1978; Itakura., et al. 1977, Goeddel, et al. 1979; Goeddel, et al. 1980) and a tryptophan (TRP) promoter system (International Application No. EP 0036776; Siebwenlist et al. 1980). While these are the most commonly used, other microbial promoters have been discovered and utilized, and details concerning their nucleotide sequences have been published, enabling a skilled worker to introduce functional promoters into plasmid vectors (Siebwenlist, et al. 1980).

In addition to prokaryotes, eukaryotic microbes such as yeast can also be used. Saccharomyces cerevisiase or common baker's yeast is the most commonly used among eukaryotic microorganisms, although a number of other strains are commonly available. For expression in Saccharomyces, the plasmid YRp7, for example, is commonly used (Stinchcomb, et al. 1979; Kingsman, et al. 1979; Tschemper, et al. 1980). This plasmid already contains the trpl gene which provides a selection marker for a mutant strain of yeast lacking the ability to grow in tryptophan, for example ATCC No. 44076 or PEP4-1 (Jones, 1977). The presence of the trp1 lesion as a characteristic of the yeast host cell genome then provides an effective environment for detecting transformation by growth in the absence of tryptophan.

Suitable promoter sequences in yeast vectors include the promoters for 3-phosphoglycerate kinase (Hitzeman., et al. 1980) or other glycolytic enzymes (Hess, et al. 1968; Holland, et al. 1978) such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also introduced into the expression vector downstream from the sequences to be expressed to provide polyadenylation of the mRNA and termination. Other promoters, which have the additional advantage of transcription controlled by growth conditions are the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Any plasmid vector containing a yeast-compatible promoter, origin or replication and termination sequences is suitable.

In addition to microorganisms, cultures of cells derived from multicellular organisms can also be used as hosts. In principle, any such cell culture is workable, whether from vertebrate or invertebrate culture. However, interest has been greatest in vertebrate cells, and propagation of vertebrate cells in culture (tissue culture) has become a routine procedure in recent years. Examples of such useful host cell lines are AtT-20, VERO and HeLa cells, Chinese hamster ovary (CHO) cell lines, and W138, BHK, COSM6, COS-7, 293 and MDCK cell lines. Expression vectors for such cells ordinarily include (if necessary) an origin of replication, a promoter located upstream of the gene to be expressed, along with any necessary ribosome binding sites, RNA splice sites, polyadenylation site, and transcriptional terminator sequences.

For use in mammalian cells, the control functions on the expression vectors are often derived from viral material. For example, commonly used promoters are derived from polyoma, Adenovirus 2, Cytomegalovirus and most frequently Simian Virus 40 (SV40). The early and late promoters of SV40 virus are particularly useful because both are obtained easily from the virus as a fragment which also contains the SV40 viral origin of replication (Fiers, et al. 1978). Smaller or larger SV40 fragments can also be used, provided there is included the approximately 250 bp sequence extending from the HindlIl site toward the BglI site located in the viral origin of replication. Further, it is also possible, and often desirable, to utilize promoter or control sequences normally associated with the desired gene sequence, provided such control sequences are compatible with the host cell systems.

An origin of replication can be provided with by construction of the vector to include an exogenous origin, such as can be derived from SV40 or other viral (e.g., Polyoma, Adeno, VSV, BPV, CMV) source, or can be provided by the host cell chromosomal replication mechanism. If the vector is integrated into the host cell chromosome, the latter is often sufficient.

In yet another embodiment, the present invention contemplates a process or method of preparing polypeptides comprising transfecting cells with polynucleotide that encode RGS9 or evectin polypeptides to produce transformed host cells; and maintaining the transformed host cells under biological conditions sufficient for expression of the polypeptide. Preferably, the transformed host cells are eukaryotic cells. Alternatively, the host cells are prokaryotic cells. More preferably, the prokaryotic cells are bacterial cells of the DH5-α strain of Escherichia coli. Even more preferably, the polynucleotide transfected into the transformed cells comprise the nucleic acid sequence of SEQ ID NO:1 or SEQ ID NO:3. Additionally, transfection is accomplished using an expression vector disclosed above.

A host cell used in the process is capable of expressing a functional, recombinant RGS9 or evectin polypeptide. A preferred host cell is a Chinese hamster ovary cell. However, a variety of cells are amenable to a process of the invention, for instance, yeast cells, human cell lines, and other eukaryotic cell lines known well to those of skill in the art.

Following transfection, the cell is maintained under culture conditions for a period of time sufficient for expression of a RGS9 or evectin polypeptide. Culture conditions are well known in the art and include ionic composition and concentration, temperature, pH and the like. Typically, transfected cells are maintained under culture conditions in a culture medium. Suitable medium for various cell types are well known in the art. In a preferred embodiment, temperature is from about 20° C. to about 50° C., more preferably from about 30° C. to about 40° C. and, even more preferably about 37° C.

pH is preferably from about a value of 6.0 to a value of about 8.0, more preferably from about a value of about 6.8 to a value of about 7.8 and, most preferably about 7.4. Osmolality is preferably from about 200 milliosmols per liter (mosm/L) to about 400 mosm/l and, more preferably from about 290 mosm/L to about 310 mosm/L. Other biological conditions needed for transfection and expression of an encoded polypeptide are well known in the art.

Transfected cells are maintained for a period of time sufficient for expression of a RGS9 or evectin polypeptide. A suitable time depends inter alia upon the cell type used and is readily determinable by a skilled artisan. Typically, maintenance time is from about 2 to about 14 days.

Recombinant RGS9 or evectin polypeptide is recovered or collected either from the transfected cells or the medium in which those cells are cultured. Recovery comprises isolating and purifying the RGS9 or evectin polypeptide. Isolation and purification techniques for polypeptides are well known in the art and include such procedures as precipitation, filtration, chromatography, electrophoresis and the like.

D. RGS9 and Evectin Antibodies

In another embodiment, the present invention provides antibodies immunoreactive with an RGS9 polypeptide or an evectin polypeptide. In other embodiments, the invention provides antibodies immunoreactive with RGS9-evectin dimers. Preferably, the antibodies of the invention are monoclonal antibodies. Additionally, the RGS9 or evectin polypeptides comprise the amino acid residue sequence of SEQ ID NO:2 or SEQ ID NO:4. Means for preparing and characterizing antibodies are well known in the art (see, e.g., Antibodies “A Laboratory Manual, E. Howell and D. Lane, Cold Spring Harbor Laboratory, 1988). In yet other embodiments, the present invention provides antibodies immunoreactive with RGS9 or evectin polynucleotides.

Briefly, a polyclonal antibody is prepared by immunizing an animal with an immunogen comprising a polypeptide or polynucleotide of the present invention, and collecting antisera from that immunized animal. A wide range of animal species can be used for the production of antisera. Typically an animal used for production of anti-antisera is a rabbit, a mouse, a rat, a hamster or a guinea pig. Because of the relatively large blood volume of rabbits, a rabbit is a preferred choice for production of polyclonal antibodies.

As is well known in the art, a given polypeptide or polynucleotide may vary in its immunogenicity. It is often necessary therefore to couple the immunogen (e g., a polypeptide or polynucleotide) of the present invention with a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers.

Means for conjugating a polypeptide or a polynucleotide to a carrier polypeptide are well known in the art and include glutaraldehyde, m-maleimidobencoyl-N-hydroxysuccinimide ester, carbodiimide and bis-biazotized benzidine.

As is also well known in the art, immunogencity to a particular immunogen can be enhanced by the use of non-specific stimulators of the immune response known as adjuvants. Exemplary and preferred adjuvants include complete Freund's adjuvant, incomplete Freund's adjuvants and aluminum hydroxide adjuvant.

The amount of immunogen used of the production of polyclonal antibodies varies inter alia, upon the nature of the immunogen as well as the animal used for immunization. A variety of routes can be used to administer the immunogen (subcutaneous, intramuscular, intradermal, intravenous and intraperitoneal. The production of polyclonal antibodies is monitored by sampling blood of the immunized animal at various points following immunization. When a desired level of immunogenicity is obtained, the immunized animal can be bled and the serum isolated and stored.

In another aspect, the present invention contemplates a process of producing an antibody immunoreactive with a RGS9 or evectin polypeptide comprising the steps of (a) transfecting recombinant host cells with a polynucleotide that encodes a RGS9 or evectin polypeptide; (b) culturing the host cells under conditions sufficient for expression of the polypeptide; (c) recovering the polypeptides; and (d) preparing the antibodies to the polypeptides. Preferably, the host cell is transfected with the polynucleotide of SEQ ID NO:1 or SEQ ID NO:3. Even more preferably, the present invention provides antibodies prepared according to the process described above.

A monoclonal antibody of the present invention can be readily prepared through use of well-known techniques such as those exemplified in U.S. Pat. No. 4,196,265, herein incorporated by reference. Typically, a technique involves first immunizing a suitable animal with a selected antigen (e.g., a polypeptide or polynucleotide of the present invention) in a manner sufficient to provide an immune response. Rodents such as mice and rats are preferred animals. Spleen cells from the immunized animal are then fused with cells of an immortal myeloma cell. Where the immunized animal is a mouse, a preferred myeloma cell is a murine NS-1 mycloma cell.

The fused spleen/myeloma cells are cultured in a selective medium to select fused spleen/myeloma cells from the parental cells. Fused cells are separated from the mixture of non-fused parental cells, e.g., by the addition of agents that block the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides. Where azaserine is used, the media is supplemented with hypoxanthine.

This culturing provides a population of hybridomas from which specific hybridomas are selected. Typically, selection of hybridomas is performed by culturing the cells by single-clone dilution in microtiter plates, followed by testing the individual clonal supernatants for reactivity with an antigen-polypeptide. The selected clones can then be propagated indefinitely to provide the monoclonal antibody.

By way of specific example, to produce an antibody of the present invention, mice are injected intraperitoneally with between about 1-200 μg of an antigen comprising a polypeptide of the present invention. B lymphocyte cells are stimulated to grow by injecting the antigen in association with an adjuvant such as complete Freund's adjuvant (a non-specific stimulator of the immune response containing killed Mycobacterium tuberculosis). At some time (e.g., at least two weeks) after the first injection, mice are boosted by injection with a second dose of the antigen mixed with incomplete Freund's adjuvant.

A few weeks after the second injection, mice are tail bled and the sera titered by immunoprecipitation against radiolabeled antigen. Preferably, the process of boosting and titering is repeated until a suitable titer is achieved. The spleen of the mouse with the highest titer is removed and the spleen lymphocytes are obtained by homogenizing the spleen with a syringe. Typically, a spleen from an immunized mouse contains approximately 5×10⁷ to 2×10⁸ lymphocytes.

Mutant lymphocyte cells known as myeloma cells are obtained from laboratory animals in which such cells have been induced to grow by a variety of well-known methods. Myeloma cells lack the salvage pathway of nucleotide biosynthesis. Because myeloma cells are tumor cells, they can be propagated indefinitely in tissue culture, and are thus denominated immortal. Numerous cultured cell lines of myeloma cells from mice and rats, such as murine NS-1 myeloma cells, have been established.

Myeloma cells are combined under conditions appropriate to foster fusion with the normal antibody-producing cells from the spleen of the mouse or rat injected with the antigen/polypeptide of the present invention. Fusion conditions include, for example, the presence of polyethylene glycol. The resulting fused cells are hybridoma cells. Like mycloma cells, hybridoma cells grow indefinitely in culture.

Hybridoma cells are separated from unfused myeloma cells by culturing in a selection medium such as HAT media (hypoxanthine, aminopterin, thymidine). Unfused myeloma cells lack the enzymes necessary to synthesize nucleotides from the salvage pathway because they are killed in the presence of aminopterin, methotrexate, or azaserine. Unfused lymphocytes also do not continue to grow in tissue culture. Thus, only cells that have successfully fused (hybridoma cells) can grow in the selection media.

Each of the surviving hybridoma cells produces a single antibody. These cells are then screened for the production of the specific antibody immunoreactive with an antigen/polypeptide of the present invention. Single cell hybridomas are isolated by limiting dilutions of the hybridomas. The hybridomas are serially diluted many times and, after the dilutions are allowed to grow, the supernatant is tested for the presence of the monoclonal antibody. The clones producing that antibody are then cultured in large amounts to produce an antibody of the present invention in convenient quantity.

By use of a monoclonal antibody of the present invention, specific polypeptides and polynucleotide of the invention can be recognized as antigens, and thus identified. Once identified, those polypeptides and polynucleotide can be isolated and purified by techniques such as antibody-affinity chromatography. In antibody-affinity chromatography, a monoclonal antibody is bound to a solid substrate and exposed to a solution containing the desired antigen. The antigen is removed from the solution through an immunospecific reaction with the bound antibody. The polypeptide or polynucleotide is then easily removed from the substrate and purified.

Additionally, examples of methods and reagents particularly amenable for use in generating and screening antibody display library can be found in, for example, U.S. Pat. No. 5,223,409; International Application No. WO 92/18619; International Application No. WO 91/17271; International Application No. WO 92/20791; International Application No. WO 92/15679; International Application No. WO 93/01288; International Application No. WO 92/01047; International Application No. WO 92/09690; International Application No. WO 90/02809.

Additionally, recombinant anti-RGS9 or -evectin antibodies, such as chimeric and humanized monoclonal antibodies, comprising both human and non-human fragments, which can be made using standard recombinant DNA techniques, are within the scope of the invention. Such chimeric and humanized monoclonal antibodies can be produced by recombinant DNA techniques known in the art, for example using methods described in PCT/US86/02269; International Application Nos. EP 184,187; EP 171,496; EP 173,494; International Application No. WO 86/01533; U.S. Pat. No. 4,816,567; and International Application No. EP 125,023.

An anti-RGS9 or -evectin antibody (e.g., monoclonal antibody) can be used to isolate RGS9 or evectin polypeptides, respectively, by standard techniques, such as affinity chromatography or immunoprecipitation. An anti-RGS9 or -evectin antibody can facilitate the purification of a natural RGS9 or evectin polypeptides from cells and recombinantly produced RGS9 or evectin polypeptide expressed in host cells. Moreover, an anti-RGS9 or -evectin antibody can be used to detect RGS9 or evectin polypeptide (e.g., in a cellular lysate or cell supernatant) in order to evaluate the abundance and pattern of expression of the RGS9 or evectin polypeptide. The detection of circulating fragments of a RGS9 or evectin polypeptide can be used to identify RGS9 or evectin polypeptide turnover in a subject. Anti-RGS9 or -evectin antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, P-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylarnine fluorescein, dansyl chloride or phycoerythrin; an example of a luminescent material includes luminol; examples of bioluminescent materials include luciferase, luciferin, and acquorin, and examples of suitable radioactive material include ¹²⁵I, ¹³¹I, ¹⁵S, ³H.

E. Transgenic Animals

In certain preferred embodiments, the invention pertains to nonhuman animals with somatic and germ cells having a functional disruption of at least one, and more preferably both, alleles of an endogenous RGS9 or evectin gene of the present invention. Accordingly, the invention provides viable animals having a mutated RGS9 or evectin gene, and thus lacking RGS9 or evectin activity. These animals will produce substantially reduced amounts of a RGS9 or evectin in response to stimuli that produce normal amounts of a RGS9 or evectin in wild type control animals. The animals of the invention are useful, for example, as standard controls by which to evaluate RGS9 or evectin inhibitors, as recipients of a normal human RGS9 or evectin gene to thereby create a model system for screening human RGS9 or evectin inhibitors in vivo, and to identify disease states for treatment with RGS9 or evectin inhibitors. The animals are also useful as controls for studying the effect of ligands on the RGS9 or evectin.

In the transgenic nonhuman animal of the invention, the RGS9 or evectin gene preferably is disrupted by homologous recombination between the endogenous allele and a mutant RGS9 or evectin polynucleotide, or portion thereof, that has been introduced into an embryonic stem cell precursor of the animal. The embryonic stem cell precursor is then allowed to develop, resulting in an animal having a functionally disrupted RGS9 or evectin gene. As used herein, a “transgenic animal” is a non-human animal, preferably a mammal, more preferably a rodent such as a rat or mouse, in which one or more of the cells of the animal include a transgene. Other examples of transgenic animals include non-human primates, sheep, dogs, cows, goats, chickens, amphibians, and the like. The animal may have one RGS9 or evectin gene allele functionally disrupted (i.e., the animal may be heterozygous for the mutation), or more preferably, the animal has both RGS9 or evectin gene alleles functionally disrupted (i.e., the animal can be homozygous for the mutation).

In one embodiment of the invention, functional disruption of both RGS9 or evectin gene alleles produces animals in which expression of the RGS9 or evectin gene product in cells of the animal is substantially absent relative to non-mutant animals. In another embodiment, the RGS9 or evectin gene alleles can be disrupted such that an altered (i.e., mutant) RGS9 or evectin gene product is produced in cells of the animal. A preferred nonhuman animal of the invention having a functionally disrupted RGS9 or evectin gene is a mouse. Given the essentially complete inactivation of RGS9 or evectin function in the homozygous animals of the invention and the about 50% inhibition of RGS9 or evectin function in the heterozygous animals of the invention, these animals are useful as positive controls against which to evaluate the effectiveness of RGS9 or evectin inhibitors. For example, a stimulus that normally induces production or activity of RGS9 or evectin can be administered to a wild type animal (i.e., an animal having a non-mutant RGS9 or evectin gene) in the presence of a RGS9 or evectin inhibitor to be tested and production or activity of RGS9 or evectin by the animal can be measured. The RGS9 or evectin response in the wild type animal can then be compared to the RGS9 or evectin response in the heterozygous and homozygous animals of the invention, similarly administered the RGS9 or evectin stimulus, to determine the percent of maximal RGS9 or evectin inhibition of the test inhibitor.

Additionally, the animals of the invention are useful for determining whether a particular disease condition involves the action of RGS9 or evectin and thus can be treated by a RGS9 or evectin inhibitor. For example, an attempt can be made to induce a disease condition in an animal of the invention having a functionally disrupted RGS9 or evectin gene. Subsequently, the susceptibility or resistance of the animal to the disease condition can be determined. A disease condition that is treatable with a RGS9 or evectin inhibitor can be identified based upon resistance of an animal of the invention to the disease condition. Another aspect of the invention pertains to a transgenic nonhuman animal having a functionally disrupted endogenous RGS9 or evectin gene but which also carries in its genome, and expresses, a transgene encoding a heterologous RGS9 or evectin (i.e., a RGS9 or evectin from another species). Preferably, the animal is a mouse and the heterologous RGS9 or evectin is a human RGS9 or evectin. An animal of the invention which has been reconstituted with human RGS9 or evectin can be used to identify agents that inhibit human RGS9 or evectin in vivo. For example, a stimulus that induces production and/or activity of RGS9 or evectin can be administered to the animal in the presence and absence of an agent to be tested and the RGS9 or evectin response in the animal can be measured. An agent that inhibits human RGS9 or evectin in vivo can be identified based upon a decreased RGS9 or evectin response in the presence of the agent compared to the RGS9 or evectin response in the absence of the agent. As used herein, a “transgene” is exogenous DNA which is integrated into the genome of a cell from which a transgenic animal develops and which remains in the genome of the mature animal, thereby directing the expression of an encoded gene product in one or more cell types or tissues of the transgenic animal.

Yet another aspect of the invention pertains to a polynucleotide construct for functionally disrupting a RGS9 or evectin gene in a host cell. The nucleic acid construct comprises: a) a nonhomologous replacement portion; b) a first homology region located upstream of the nonhomologous replacement portion, the first homology region having a nucleotide sequence with substantial identity to a first RGS9 or evectin gene sequence; and c) a second homology region located downstream of the nonhomologous replacement portion, the second homology region having a nucleotide sequence with substantial identity to a second RGS9 or evectin gene sequence, the second RGS9 or evectin gene sequence having a location downstream of the first RGS9 or evectin gene sequence in a naturally occurring endogenous RGS9 or evectin gene. Additionally, the first and second homology regions are of sufficient length for homologous recombination between the nucleic acid construct and an endogenous RGS9 or evectin gene in a host cell when the nucleic acid molecule is introduced into the host cell. As used herein, a “homologous recombinant animal” is a non-human animal, preferably a mammal, more preferably a mouse, in which an endogenous RGS9 or evectin gene has been altered by homologous recombination between the endogenous gene and an exogenous DNA molecule introduced into a cell of the animal, e.g., an embryonic cell of the animal, prior to development of the animal.

In a preferred embodiment, the nonhomologous replacement portion comprises a positive selection expression cassette, preferably including a neomycin phosphotransferase gene operatively linked to a regulatory element(s). In another preferred embodiment, the nucleic acid construct also includes a negative selection expression cassette distal to either the upstream or downstream homology regions. A preferred negative selection cassette includes a herpes simplex virus thymidine kinase gene operatively linked to a regulatory element(s). Another aspect of the invention pertains to recombinant vectors into which the nucleic acid construct of the invention has been incorporated.

Yet another aspect of the invention pertains to host cells into which the nucleic acid construct of the invention has been introduced to thereby allow homologous recombination between the nucleic acid construct and an endogenous RGS9 or evectin gene of the host cell, resulting in functional disruption of the endogenous RGS9 or evectin gene. The host cell can be a mammalian cell that normally expresses RGS9 or evectin, such as a human neuron, or a pluripotent cell, such as a mouse embryonic stem cell. Further development of an embryonic stem cell into which the nucleic acid construct has been introduced and homologously recombined with the endogenous RGS9 or evectin gene produces a transgenic nonhuman animal having cells that are descendant from the embryonic stem cell and thus carry the RGS9 or evectin gene disruption in their genome. Animals that carry the RGS9 or evectin gene disruption in their germline can then be selected and bred to produce animals having the RGS9 or evectin gene disruption in all somatic and germ cells. Such mice can then be bred to homozygosity for the RGS9 or evectin gene disruption.

It is contemplated that in some instances the genome of a transgenic animal of the present invention will have been altered through the stable introduction of one or more of the RGS9 or evectin polynucleotide compositions described herein, either native, synthetically modified or mutated. As described herein, a “transgenic animal” refers to any animal, preferably a non-human mammal (e.g. mouse, rat, rabbit, squirrel, hamster, rabbits, guinea pigs, pigs, micro-pigs, prairie, baboons, squirrel monkeys and chimpanzees, etc), bird or an amphibian, in which one or more cells contain heterologous nucleic acid introduced by way of human intervention, such as by transgenic techniques well known in the art. The nucleic acid is introduced into the cell, directly or indirectly, by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA.

The host cells of the invention can also be used to produce non-human transgenic animals. The non-human transgenic animals can be used in screening assays designed to identify agents or compounds, e.g., drugs, pharmaceuticals, etc., which are capable of ameliorating detrimental symptoms of selected disorders such as nervous system disorders, e.g., psychiatric disorders or disorders affecting circadian rhythms and the sleep-wake cycle. For example, in one embodiment, a host cell of the invention is a fertilized oocyte or an embryonic stem cell into which RGS9 or evectin polypeptide-coding sequences have been introduced. Such host cells can then be used to create non-human transgenic animals in which exogenous RGS9 or evectin gene sequences have been introduced into their genome or homologous recombinant animals in which endogenous RGS9 or evectin gene sequences have been altered. Such animals are useful for studying the function and/or activity of a RGS9 or evectin polypeptide and for identifying and/or evaluating modulators of RGS9 or evectin polypeptide activity.

A transgenic animal of the invention can be created by introducing RGS9 or evectin polypeptide encoding nucleic acid into the male pronuclei of a fertilized oocyte, e.g., by microinjection, retroviral infection, and allowing the oocyte to develop in a pseudopregnant female foster animal. The human RGS9 or evectin cDNA sequence of SEQ ID NO:1 or SEQ ID NO:3, respectively, can be introduced as a transgene into the genome of a non-human animal.

Moreover, a non-human homologue of the human RGS9 or evectin gene, such as a mouse RGS9 or evectin gene, can be isolated based on hybridization to the human RGS9 or evectin cDNA (described above) and used as a transgene. Intronic sequences and polyadenylation signals can also be included in the transgene to increase the efficiency of expression of the transgene. A tissue-specific regulatory sequence(s) can be operably linked to the RGS9 or evectin transgene to direct expression of a RGS9 or evectin polypeptide to particular cells. Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, 1986. Similar methods are used for production of other transgenic animals. A transgenic founder animal can be identified based upon the presence of the RGS9 or evectin transgene in its genome and/or expression of RGS9 or evectin mRNA in tissues or cells of the animals. A transgenic founder animal can then be used to breed additional animals carrying the transgene. Moreover, transgenic animals carrying a transgene encoding a RGS9 or evectin polypeptide can further be bred to other transgenic animals carrying other transgenes.

To create a homologous recombinant animal, a vector is prepared which contains at least a fragment of a RGS9 or evectin gene into which a deletion, addition or substitution has been introduced to thereby alter, e.g., functionally disrupt, the RGS9 or evectin gene. The RGS9 or evectin gene can be a human gene (e.g. from a human genomic clone isolated from a human genomic library screened with the cDNA of SEQ ID NO:1 or SEQ ID NO:3), but more preferably is a non-human homologue of a human RGS9 or evectin gene. For example, a mouse RGS9 or evectin gene can be isolated from a mouse genomic DNA library using the RGS9 or evectin cDNA of SEQ ID NO: 1 or SEQ ID NO:3, respectively, as a probe. The mouse RGS9 or evectin gene then can be used to construct a homologous recombination vector suitable for altering an endogenous RGS9 or evectin gene in the mouse genome. In a preferred embodiment, the vector is designed such that, upon homologous recombination, the endogenous RGS9 or evectin gene is functionally disrupted (i.e., no longer encodes a functional protein; also referred to as a “knock out” vector.

Alternatively, the vector can be designed such that, upon homologous recombination, the endogenous RGS9 or evectin gene is mutated or otherwise altered but still encodes functional protein (e.g., the upstream regulatory region can be altered to thereby alter the expression of the endogenous RGS9 or evectin polypeptide). In the homologous recombination vector, the altered fragment of the RGS9 or evectin gene is flanked at its 5′ and 3′ ends by additional nucleic acid of the RGS9 or evectin to allow for homologous recombination to occur between the exogenous RGS9 or evectin gene carried by the vector and an endogenous RGS9 or evectin gene in an embryonic stem cell. The additional flanking RGS9 or evectin nucleic acid is of sufficient length for successful homologous recombination with the endogenous gene.

Typically, several kilobases of flanking DNA (both at the 5′ and 3′ ends) are included in the vector (see e.g., Thomas and Capecchi, 1987, for a description of homologous recombination vectors). The vector is introduced into an embryonic stem cell line (e.g., by electroporation) and cells in which the introduced RGS9 or evectin gene has homologously recombined with the endogenous RGS9 or evectin gene are selected (see e.g., Li et al., 1992). The selected cells are then injected into a blastocyst of an animal (e.g., a mouse) to form aggregation chimeras (see e.g., Bradley, 1987, pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, 1991; and in PCT International Publication Nos. WO 90/11354; WO 91/01140; WO 92/0968; and WO 93/04169.

In another embodiment, transgenic non-human animals can be produced which contain selected systems which allow for regulated expression of the transgene. One example of such a system is the cre/loxP recombinase system of bacteriophage PL. For a description of the cre/loxP recombinase system, see, e.g., Lakso et al., 1992. Another example of a recombinase system is the FLP recombinase system of Saccharomyces cerevisiae (O'Gon-nan et al., 1991). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al., 1997, and PCT International Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyst and then transferred to pseudopregnant female foster animal. The offspring borne of this female foster animal will be a clone of the animal from which the cell, e.g., the somatic cell, is isolated.

F. Uses and Methods of the Invention

The nucleic acid molecules, polypeptides, polypeptide homologues, modulators, antibodies, vectors and host cells described herein can be used in one or more of the following methods: a) drug screening assays; b) diagnostic assays particularly in disease identification, allelic screening and pharmocogenetic testing; c) methods of treatment; d) pharmacogenomics; and e) monitoring of effects during clinical trials. A polypeptide of the invention can be used as a drug target for developing agents to modulate the activity of a RGS9-evectin polypeptide dimer. The isolated nucleic acid molecules of the invention can be used to express RGS9 and evectin polypeptide (e.g., via a recombinant expression vector in a host cell or in gene therapy applications), to detect RGS9 and evectin mRNA (e.g., in a biological sample) or a naturally occurring or recombinantly generated genetic mutation in a RGS9 or evectin gene, and to modulate RGS9 or evectin polypeptide activity, as described further below. In addition, the RGS9 and evectin polypeptides can be used to screen drugs or compounds which modulate polypeptide activity. Moreover, the anti-RGS9 or evectin antibodies of the invention can be used to detect and isolate a RGS9 or evectin polypeptide, particularly fragments of a RGS9 and evectin polypeptides present in a biological sample, and to modulate RGS9 and evectin polypeptide activity.

Drug Screening Assays

The invention provides methods for identifying compounds or agents that can be used to treat disorders characterized by (or associated with) aberrant or abnormal RGS9-evectin nucleic acid expression and/or abnormal RGS9-evectin polypeptide activity. These methods are also referred to herein as drug screening assays and typically include the step of screening a candidate/test compound or agent to identify compounds that are an agonist or antagonist of a RGS9 or evectin polypeptide, and specifically for the ability to interact with (e.g., bind to) a RGS9 or evectin polypeptide, to modulate the interaction of a RGS9 or evectin polypeptide and a target molecule, and/or to modulate RGS9 or evectin nucleic acid expression and/or RGS9 or evectin polypeptide activity. Candidate/test compounds or agents which have one or more of these abilities can be used as drugs to treat disorders characterized by aberrant or abnormal RGS9 or evectin nucleic acid expression and/or RGS9 or evectin polypeptide activity. Candidate/test compounds include, for example, 1) peptides such as soluble peptides, including Ig-tailed fusion peptides and members of random peptide libraries and combinatorial chemistry-derived molecular libraries made of D- and/or L-configuration amino acids; 2) phosphopeptides (e.g., members of random and partially degenerate, directed phosphopeptide libraries, see, e.g., Songyang et al., 1993; 3) antibodies (e.g., polyclonal, monoclonal, humanized, anti-idiotypic, chimeric, and single chain antibodies as well as Fab, F(ab′)2, Fab expression library fragments, and epitope-binding fragments of antibodies); and 4) small organic and inorganic molecules (e.g., molecules obtained from combinatorial and natural product libraries). In one embodiment, the invention provides assays for screening candidate/test compounds which interact with (e.g., bind to) a RGS9 or evectin polypeptide. Typically, the assays are recombinant cell based or cell-free assays which include the steps of combining a cell expressing a RGS9 polypeptide fragment, an evectin polypeptide fragment or an RGS9-evectin dimer polypeptide or a bioactive fragment thereof, or an isolated RGS9 polypeptide fragment, an evectin polypeptide fragment or an RGS9-evectin dimer polypeptide, and a candidate/test compound, e.g., under conditions which allow for interaction of (e.g., binding of) the candidate/test compound to the RGS9 polypeptide fragment, an evectin polypeptide fragment or an RGS9-evectin dimer polypeptide or fragment thereof to form a complex, and detecting the formation of a complex, in which the ability of the candidate compound to interact with (e.g., bind to) the RGS9 polypeptide fragment, an evectin polypeptide fragment or an RGS9-evectin dimer polypeptide or fragment thereof is indicated by the presence of the candidate compound in the complex. Formation of complexes between the RGS9 polypeptide fragment, an evectin polypeptide fragment or an RGS9-evectin dimer polypeptide and the candidate compound can be detected using competition binding assays, and can be quantitated, for example, using standard immunoassays.

In another embodiment, the invention provides screening assays to identify candidate/test compounds which modulate (e.g., stimulate or inhibit) the interaction (and most likely polypeptide activity as well) between a RGS9 polypeptide fragment, an evectin polypeptide fragment or an RGS9-evectin dimer polypeptide and a molecule (target molecule) with which the RGS9 polypeptide fragment, an evectin polypeptide fragment or an RGS9-evectin dimer polypeptide normally interacts. Examples of such target molecules include proteins in the same signaling path as the RGS9 polypeptide fragment, an evectin polypeptide fragment or an RGS9-evectin dimer polypeptide, e.g., proteins which may function upstream (including both stimulators and inhibitors of activity) or downstream of the RGS9 polypeptide fragment, an evectin polypeptide fragment or an RGS9-evectin dimer polypeptide in, for example, a cognitive function signaling pathway or in a pathway involving RGS9 polypeptide fragment, an evectin polypeptide fragment or an RGS9-evectin dimer polypeptide activity, e.g., a G protein or other interactor involved in cAMP or phosphatidylinositol turnover, and/or adenylyl cyclase or phospholipase C activation or ion channel modulation. Typically, the assays are recombinant cell based assays which include the steps of combining a cell expressing a RGS9 polypeptide fragment, an evectin polypeptide fragment or an RGS9-evectin dimer polypeptide, or a bioactive fragment thereof, a RGS9 polypeptide fragment, an evectin polypeptide fragment or an RGS9-evectin dimer polypeptide target molecule (e.g., a RGS9 polypeptide fragment, an evectin polypeptide fragment or an RGS9-evectin dimer ligand) and a candidate/test compound, e.g., under conditions wherein but for the presence of the candidate compound, the RGS9 polypeptide fragment, an evectin polypeptide fragment or an RGS9-evectin dimer polypeptide or biologically active fragment thereof interacts with (e.g., binds to) the target molecule, and detecting the formation of a complex which includes the RGS9 polypeptide fragment, an evectin polypeptide fragment or an RGS9-evectin dimer polypeptide and the target molecule or detecting the interaction/reaction of the RGS9 polypeptide fragment, an evectin polypeptide fragment or an RGS9-evectin dimer polypeptide and the target molecule.

Detection of complex formation can include direct quantitation of the complex by, for example, measuring inductive effects of the RGS9 polypeptide fragment, an evectin polypeptide fragment or an RGS9-evectin dimer polypeptide. A statistically significant change, such as a decrease, in the interaction of the RGS9 polypeptide fragment, an evectin polypeptide fragment or an RGS9-evectin dimer polypeptide and target molecule (e.g., in the formation of a complex between the RGS9 polypeptide fragment, an evectin polypeptide fragment or an RGS9-evectin dimer polypeptide and the target molecule) in the presence of a candidate compound (relative to what is detected in the absence of the candidate compound) is indicative of a modulation (e.g., stimulation or inhibition) of the interaction between the RGS9 polypeptide fragment, an evectin polypeptide fragment or an RGS9-evectin dimer polypeptide and the target molecule. Modulation of the formation of complexes between the RGS9 polypeptide fragment, an evectin polypeptide fragment or an RGS9-evectin dimer polypeptide and the target molecule can be quantitated using, for example, an immunoassay.

To perform cell free drug screening assays, it is desirable to immobilize either the RGS9 polypeptide fragment, an evectin polypeptide fragment or an RGS9-evectin dimer polypeptide or its target molecule to facilitate separation of complexes from uncomplexed forms of one or both of the proteins, as well as to accommodate automation of the assay. Interaction (e.g., binding of) of the RGS9 polypeptide fragment, an evectin polypeptide fragment or an RGS9-evectin dimer polypeptide to a target molecule, in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows the protein to be bound to a matrix. For example, glutathione-S-transferase/RGS9 polypeptide fragment, an evectin polypeptide fragment or an RGS9-evectin dimer fusion proteins can be adsorbed onto glutathione sepharose beads (Sigma Chemical, St. Louis, Mo.) or glutathione derivatized microtitre plates, which are then combined with the cell lysates (e.g., ³⁵S labeled) and the candidate compound, and the mixture incubated under conditions conducive to complex formation (e.g., at physiological conditions for salt and pH). Following incubation, the beads are washed to remove any unbound label, and the matrix immobilized and radiolabel determined directly, or in the supernatant after the complexes are dissociated. Alternatively, the complexes can be dissociated from the matrix, separated by SDS-PAGE, and the level of RGS9 polypeptide fragment, an evectin polypeptide fragment or an RGS9-evectin dimer-binding protein found in the bead fraction quantitated from the gel using standard electrophoretic techniques.

Other techniques for immobilizing proteins on matrices can also be used in the drug screening assays of the invention. For example, either the RGS9 polypeptide fragment, an evectin polypeptide fragment or an RGS9-evectin dimer polypeptide or its target molecule can be immobilized utilizing conjugation of biotin and streptavidin. Biotinylated RGS9 polypeptide fragment, an evectin polypeptide fragment or an RGS9-evectin dimer polypeptide molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with a RGS9 polypeptide fragment, an evectin polypeptide fragment or an RGS9-evectin dimer polypeptide but which do not interfere with binding of the protein to its target molecule can be derivatized to the wells of the plate, and RGS9 polypeptide fragment, an evectin polypeptide fragment or an RGS9-evectin dimer polypeptide trapped in the wells by antibody conjugation. As described above, preparations of a RGS9 polypeptide fragment, an evectin polypeptide fragment or an RGS9-evectin dimer-binding protein and a candidate compound are incubated in the RGS9 polypeptide fragment, an evectin polypeptide fragment or an RGS9-evectin dimer polypeptide-presenting wells of the plate, and the amount of complex trapped in the well can be quantitated. Methods for detecting such complexes, in addition to those described above for the GST-immobilized complexes, include immunodetection of complexes using antibodies reactive with the RGS9 polypeptide fragment, an evectin polypeptide fragment or an RGS9-evectin dimer polypeptide target molecule, or which are reactive with RGS9 polypeptide fragment, an evectin polypeptide fragment or an RGS9-evectin dimer polypeptide and compete with the target molecule; as well as enzyme-linked assays which rely on detecting an enzymatic activity associated with the target molecule.

In yet another embodiment, the invention provides a method for identifying a compound (e.g., a screening assay) capable of use in the treatment of a disorder characterized by (or associated with) aberrant or abnormal RGS9 or evectin nucleic acid expression or RGS9 or evectin polypeptide activity. This method typically includes the step of assaying the ability of the compound or agent to modulate the expression of the RGS9 or evectin nucleic acid or the activity of the RGS9 or evectin polypeptide thereby identifying a compound for treating a disorder characterized by aberrant or abnormal RGS9 or evectin nucleic acid expression or RGS9 or evectin polypeptide activity. Methods for assaying the ability of the compound or agent to modulate the expression of the RGS9 or evectin nucleic acid or activity of the RGS9 or evectin polypeptide are typically cell-based assays. For example, cells which are sensitive to ligands which transduce signals via a pathway involving a RGS9 or evectin polypeptide can be induced to overexpress a RGS9 or evectin polypeptide in the presence and absence of a candidate compound.

Candidate compounds which produce a statistically significant change in RGS9 or evectin polypeptide-dependent responses (either stimulation or inhibition) can be identified. In one embodiment, expression of the RGS9 or evectin nucleic acid or activity of a RGS9 or evectin polypeptide is modulated in cells and the effects of candidate compounds on the readout of interest (such as cAMP or phosphatidylinositol turnover) are measured. For example, the expression of genes which are up- or down-regulated in response to a RGS9 or evectin polypeptide-dependent signal cascade can be assayed. In preferred embodiments, the regulatory regions of such genes, e.g., the 5′ flanking promoter and enhancer regions, are operably linked to a detectable marker (such as luciferase) which encodes a gene product that can be readily detected. Phosphorylation of a RGS9 or evectin polypeptide or RGS9 or evectin polypeptide target molecules can also be measured, for example, by immunoblotting.

Alternatively, modulators of RGS9 or evectin gene expression (e.g., compounds which can be used to treat a disorder characterized by aberrant or abnormal RGS9 or evectin nucleic acid expression or RGS9 or evectin polypeptide activity) can be identified in a method wherein a cell is contacted with a candidate compound and the expression of RGS9 or evectin mRNA or protein in the cell is determined. The level of expression of RGS9 or evectin mRNA or protein in the presence of the candidate compound is compared to the level of expression of RGS9 or evectin mRNA or protein in the absence of the candidate compound. The candidate compound can then be identified as a modulator of RGS9 or evectin nucleic acid expression based on this comparison and be used to treat a disorder characterized by aberrant RGS9 or evectin nucleic acid expression. For example, when expression of RGS9 or evectin mRNA or protein is greater (statistically significantly greater) in the presence of the candidate compound than in its absence, the candidate compound is identified as a stimulator of RGS9 or evectin nucleic acid expression. Alternatively, when RGS9 or evectin nucleic acid expression is less (statistically significantly less) in the presence of the candidate compound than in its absence, the candidate compound is identified as an inhibitor of RGS9 or evectin nucleic acid expression. The level of RGS9 or evectin nucleic acid expression in the cells can be determined by methods described herein for detecting RGS9 or evectin mRNA or protein.

In certain aspects of the invention, RGS9 or evectin polypeptides or portions thereof can be used as “bait proteins” in a two-hybrid assay or three-hybrid assay (see, e.g., U.S. Pat. No. 5,283,317; U.S. Statutory Invention Registration No. H1,892; Zervos et al., 1993; Madura et al., 1993; Bartel et al., 1993(b); Iwabuchi et al., 1993; International Application No. WO94/10300), to identify other proteins, which bind to or interact with RGS9 and/or evectin and are involved in RGS9 and/or evectin activity. Such RGS9 or evectin-binding proteins are also likely to be involved in the propagation of signals by the RGS9 or evectin polypeptides or RGS9 or evectin targets as, for example, downstream elements of a G-protein-mediated signaling pathway. Alternatively, such RGS9 or evectin-binding proteins may be RGS9 or evectin inhibitors.

Thus, in certain embodiments, the invention contemplates determining protein:protein interactions. The yeast two-hybrid system is extremely useful for studying protein:protein interactions. Variations of the system are available for screening yeast phagemid (Harper et al., 1993; Elledge et al., 1991) or plasmid (Bartel et al., 1993(b), Bartel 1993(a); Finley and Brent, 1994) cDNA libraries to clone interacting proteins, as well as for studying known protein pairs. Recently, a two-hybrid method for high volume screening for specific inhibitors of protein:protein interactions and a two-hybrid screen that identifies many different interactions between protein pairs at once have been described (see, U.S. Statutory Invention Registration No. H1,892).

The success of the two-hybrid system relies upon the fact that the DNA binding and polymerase activation domains of many transcription factors, such as GAL4, can be separated and then rejoined to restore functionality (Morin et al., 1993). Briefly, the assay utilizes two different DNA constructs. In one construct, the gene that codes for a RGS9 or evectin polypeptide is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a RGS9 or evectin dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the RGS9 or evectin polypeptide.

Modulators of RGS9 or evectin polypeptide activity and/or RGS9 or evectin nucleic acid expression identified according to these drug screening assays can be used to treat, for example, nervous system disorders. These methods of treatment include the steps of administering the modulators of RGS9 or evectin polypeptide activity and/or nucleic acid expression, e.g., in a pharmaceutical composition as described herein, to a subject in need of such treatment, e.g., a subject with a disorder described herein.

Diagnostic Assays

The invention further provides a method for detecting the presence of a RGS9 or evectin polypeptide or RGS9 or evectin nucleic acid molecule, or fragment thereof, in a biological sample. The method involves contacting the biological sample with a compound or an agent capable of detecting RGS9 or evectin polypeptide or mRNA such that the presence of RGS9 or evectin polypeptide/encoding nucleic acid molecule is detected in the biological sample. A preferred agent for detecting RGS9 or evectin mRNA is a labeled or labelable nucleic acid probe capable of hybridizing to RGS9 or evectin mRNA. The nucleic acid probe can be, for example, the full-length RGS9 or evectin cDNA of SEQ ID NO: 1 or SEQ ID NO: 3 or a fragment thereof, such as an oligonucleotide of at least 15, 30, 50, 100, 250 or 500 nucleotides in length and sufficient to specifically hybridize under stringent conditions to RGS9 or evectin mRNA. A preferred agent for detecting RGS9 or evectin polypeptide is a labeled or labelable antibody capable of binding to RGS9 or evectin polypeptide or dimer of RGS9-evectin. Antibodies can be polyclonal, or more preferably, monoclonal. An intact antibody, or a fragment thereof (e.g., Fab or F(ab′)2) can be used. The term “labeled or labelable,” with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect RGS9 or evectin mRNA or protein in a biological sample in vitro as well as in vivo. For example, in vitro techniques for detection of RGS9 or evectin mRNA include Northern hybridizations and in situ hybridizations. In vitro techniques for detection of RGS9 or evectin polypeptide include enzyme linked immunosorbent assays (ELISAs), Western blots, immunoprecipitations and immunofluorescence. Alternatively, RGS9 or evectin polypeptide can be detected in vivo in a subject by introducing into the subject a labeled anti-RGS9 or evectin antibody. For example, the antibody can be labeled with a radioactive marker whose presence and location in a subject can be detected by standard imaging techniques. Particularly useful are methods which detect the allelic variant of a RGS9 or evectin polypeptide expressed in a subject and methods which detect fragments of a RGS9 or evectin polypeptide in a sample.

The invention also encompasses kits for detecting the presence of a RGS9 or evectin polypeptide in a biological sample. For example, the kit can comprise reagents such as a labeled or labelable compound or agent capable of detecting RGS9 or evectin polypeptide or mRNA in a biological sample; means for determining the amount of RGS9 or evectin polypeptide in the sample; and means for comparing the amount of RGS9 or evectin polypeptide in the sample with a standard. The compound or agent can be packaged in a suitable container. The kit can further comprise instructions for using the kit to detect RGS9 or evectin mRNA or protein.

The methods of the invention can also be used to detect naturally occurring genetic mutations in a RGS9 or evectin gene, thereby determining if a subject with the mutated gene is at risk for a disorder characterized by aberrant or abnormal RGS9 or evectin nucleic acid expression or RGS9 or evectin polypeptide activity as described herein. In preferred embodiments, the methods include detecting, in a sample of cells from the subject, the presence or absence of a genetic mutation characterized by at least one of an alteration affecting the integrity of a gene encoding a RGS9 or evectin polypeptide, or the misexpression of the RGS9 or evectin gene. For example, such genetic mutations can be detected by ascertaining the existence of at least one of 1) a deletion of one or more nucleotides from a RGS9 or evectin gene; 2) an addition of one or more nucleotides to a RGS9 or evectin gene; 3) a substitution of one or more nucleotides of a RGS9 or evectin gene, 4) a chromosomal rearrangement of a RGS9 or evectin gene; 5) an alteration in the level of a messenger RNA transcript of a RGS9 or evectin gene, 6) aberrant modification of a RGS9 or evectin gene, such as of the methylation pattern of the genomic DNA, 7) the presence of a non-wild type splicing pattern of a messenger RNA transcript of a RGS9 or evectin gene, 8) a non-wild type level of a RGS9 or evectin-protein, 9) allelic loss of a RGS9 or evectin gene, and 10) inappropriate post-translational modification of a RGS9 or evectin-protein. As described herein, there are a large number of assay techniques known in the art that can be used for detecting mutations in a RGS9 or evectin gene.

In certain embodiments, detection of the mutation involves the use of a probe/primer in a polymerase chain reaction (PCR) (see, e.g. U.S. Pat. No. 4,683,195 and U.S. Pat. No. 4,683,202), such as anchor PCR or RACE PCR, or, alternatively, in a ligation chain reaction (LCR), the latter of which can be particularly useful for detecting point mutations in the RGS9 or evectin-gene (see Abravaya et al., 1995). This method can include the steps of collecting a sample of cells from a patient, isolating nucleic acid (e.g., genomic, mRNA or both) from the cells of the sample, contacting the nucleic acid sample with one or more primers which specifically hybridize to a RGS9 or evectin gene under conditions such that hybridization and amplification of the RGS9 or evectin-gene (if present) occurs, and detecting the presence or absence of an amplification product, or detecting the size of the amplification product and comparing the length to a control sample.

In an alternative embodiment, mutations in a RGS9 or evectin gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see U.S. Pat. No. 5,498,531 hereby incorporated by reference in its entirety) can be used to score for the presence of specific mutations by development or loss of a ribozyme cleavage site. In yet another embodiment, any of a variety of sequencing reactions known in the art can be used to directly sequence the RGS9 or evectin gene and detect mutations by comparing the sequence of the sample RGS9 or evectin gene with the corresponding wild-type (control) sequence. Examples of sequencing reactions include those based on techniques developed by Maxim and Gilbert (1977) or Sanger (1977). A variety of automated sequencing procedures can be utilized when performing the diagnostic assays, including sequencing by mass spectrometry (see, e.g., International Application No. WO 94/1610 1; Cohen et al., 1996; and Griffin et al. 1993).

Other methods for detecting mutations in the RGS9 or evectin gene include methods in which protection from cleavage agents is used to detect mismatched bases in RNA/RNA or RNA/DNA duplexes (Myers et al., 1985 (b); Cotton et al., 1988; Saleeba et al., 1992), electrophoretic mobility of mutant and wild type nucleic acid is compared (Orita et al., 1989; Cotton, 1993; and Hayashi, 1992), and movement of mutant or wild-type fragments in polyacrylamide gels containing a gradient of denaturant is assayed using denaturing gradient gel electrophoresis (Myers et al., 1985(a)). Examples of other techniques for detecting point mutations include, selective oligonucleotide hybridization, selective amplification, and selective primer extension.

Methods of Treatment

Another aspect of the invention pertains to methods for treating a subject, e.g., a human, having a disease or disorder characterized by (or associated with) aberrant or abnormal RGS9 or evectin nucleic acid expression and/or RGS9 or evectin polypeptide activity. These methods include the step of administering a RGS9 or evectin polypeptide/gene modulator (agonist or antagonist) to the subject such that treatment occurs. The language “aberrant or abnormal RGS9 or evectin polypeptide expression” refers to expression of a non-wild-type RGS9 or evectin polypeptide or a non-wild-type level of expression of a RGS9 or evectin polypeptide. Aberrant or abnormal RGS9 or evectin polypeptide activity refers to a non-wild-type RGS9 or evectin polypeptide activity or a non-wild-type level of RGS9 or evectin polypeptide activity. As the RGS9 or evectin polypeptide is involved in a pathway involving signaling within cells, aberrant or abnormal RGS9 or evectin polypeptide activity or expression interferes with the normal regulation of functions mediated by RGS9 or evectin polypeptide signaling, and in particular brain cells. The terms “treating” or “treatment,” as used herein, refer to reduction or alleviation of at least one adverse effect or symptom of a disorder or disease, e.g., a disorder or disease characterized by or associated with abnormal or aberrant RGS9 or evectin polypeptide activity or RGS9 or evectin nucleic acid expression.

As used herein, a RGS9 or evectin polypeptide/gene modulator is a molecule which can modulate RGS9 or evectin nucleic acid expression and/or RGS9 or evectin polypeptide activity. For example, a RGS9 or evectin gene or protein modulator can modulate, e.g., upregulate (activate/agonize) or downregulate (suppress/antagonize), RGS9 or evectin nucleic acid expression. In another example, a RGS9 or evectin polypeptide/gene modulator can modulate (e.g., stimulate/agonize or inhibit/antagonize) RGS9 or evectin polypeptide activity. If it is desirable to treat a disorder or disease characterized by (or associated with) aberrant or abnormal (non-wild-type) RGS9 or evectin nucleic acid expression and/or RGS9 or evectin polypeptide activity by inhibiting RGS9 or evectin nucleic acid expression, a RGS9 or evectin modulator can be an antisense molecule, e.g., a ribozyme, as described herein. Examples of antisense molecules which can be used to inhibit RGS9 or evectin nucleic acid expression include antisense molecules which are complementary to a fragment of the 5′ untranslated region of SEQ ID NO: 1 or SEQ ID NO: 3, which also includes the start codon and antisense molecules which are complementary to a fragment of a 3′ untranslated region of SEQ ID NO: 1 or SEQ ID NO: 3.

A RGS9 or evectin modulator that inhibits RGS9 or evectin nucleic acid expression can also be a small molecule or other drug, e.g., a small molecule or drug identified using the screening assays described herein, which inhibits RGS9 or evectin nucleic acid expression. If it is desirable to treat a disease or disorder characterized by (or associated with) aberrant or abnormal (non-wild-type) RGS9 or evectin nucleic acid expression and/or RGS9 or evectin polypeptide activity by stimulating RGS9 or evectin nucleic acid expression, a RGS9 or evectin modulator can be, for example, a nucleic acid molecule encoding a RGS9 or evectin polypeptide (e.g., a nucleic acid molecule comprising a nucleotide sequence homologous to the nucleotide sequence of SEQ ID NO: 1 or SEQ ID NO: 3, or a small molecule or other drug, e.g., a small molecule (peptide) or drug identified using the screening assays described herein, which stimulates RGS9 or evectin nucleic acid expression.

Alternatively, if it is desirable to treat a disease or disorder characterized by (or associated with) aberrant or abnormal (non-wild-type) RGS9 or evectin nucleic acid expression and/or RGS9 or evectin polypeptide activity by inhibiting RGS9 or evectin polypeptide activity, a RGS9 or evectin modulator can be an anti-RGS9 or evectin antibody or a small molecule or other drug, e.g., a small molecule or drug identified using the screening assays described herein, which inhibits RGS9 or evectin polypeptide activity. If it is desirable to treat a disease or disorder characterized by (or associated with) aberrant or abnormal (non-wild-type) RGS9 or evectin nucleic acid expression and/or RGS9 or evectin polypeptide activity by stimulating RGS9 or evectin polypeptide activity, a RGS9 or evectin modulator can be an active RGS9 or evectin polypeptide or fragment thereof (e.g., a RGS9 or evectin polypeptide or fragment thereof having an amino acid sequence which is homologous to the amino acid sequence of SEQ ID NO:2 or SEQ ID NO: 4, or a fragment thereof) or a small molecule or other drug, e.g., a small molecule or drug identified using the screening assays described herein, which stimulates RGS9 or evectin polypeptide activity.

Other aspects of the invention pertain to methods for modulating a RGS9 or evectin polypeptide mediated cell activity. These methods include contacting the cell with an agent (or a composition which includes an effective amount of an agent) which modulates RGS9 or evectin polypeptide activity or RGS9 or evectin nucleic acid expression such that a RGS9 or evectin polypeptide mediated cell activity is altered relative to normal levels (for example, cAMP or phosphatidylinositol metabolism). As used herein, “a RGS9 or evectin polypeptide mediated cell activity” refers to a normal or abnormal activity or function of a cell. Examples of RGS9 or evectin polypeptide mediated cell activities include phosphatidylinositol turnover, cAMP turnover, production or secretion of molecules, such as proteins, contraction, proliferation, migration, differentiation, and cell survival. In a preferred embodiment, the cell is neural cell of the brain, e.g., a hippocampal cell. The term “altered” as used herein refers to a change, e.g., an increase or decrease, of a cell associated activity particularly cAMP or phosphatidylinositol turnover, and adenylyl cyclase or phospholipase C activation.

In one embodiment, the agent stimulates RGS9 or evectin polypeptide activity or RGS9 or evectin nucleic acid expression. In another embodiment, the agent inhibits RGS9 or evectin polypeptide activity or RGS9 or evectin nucleic acid expression. These modulatory methods can be performed in vitro (e.g., by culturing the cell with the agent) or, alternatively, in vivo (e.g., by administering the agent to a subject). In a preferred embodiment, the modulatory methods are performed in vivo, i.e., the cell is present within a subject, e.g., a mammal, e.g., a human, and the subject has a disorder or disease characterized by or associated with abnormal or aberrant RGS9 or evectin polypeptide activity or RGS9 or evectin nucleic acid expression.

A nucleic acid molecule, a protein, a RGS9 or evectin modulator, a compound etc. used in the methods of treatment can be incorporated into an appropriate pharmaceutical composition described below and administered to the subject through a route which allows the molecule, protein, modulator, or compound etc. to perform its intended function.

Disorders involving the brain include, but are limited to, disorders involving neurons, and disorders involving glia, such as astrocytes, oligodendrocytes, ependymal cells, and microglia; cerebral edema, raised intracranial pressure and herniation, and hydrocephalus; malformations and developmental diseases, such as neural tube defects, forebrain anomalies, posterior fossa anomalies, and syringomyelia and hydromyelia; perinatal brain injury; cerebrovascular diseases, such as those related to hypoxia, ischemia, and infarction, including hypotension, hypoperfusion, and low-flow states—global cerebral ischemia and focal cerebral ischemia—infarction from obstruction of local blood supply, intracranial hemorrhage, including intracerebral (intraparenchymal) hemorrhage, subarachnoid hemorrhage and ruptured berry aneurysms, and vascular malformations, hypertensive cerebrovascular disease, including lacunar infarcts, slit hemorrhages, and hypertensive encephalopathy; infections, such as acute meningitis, including acute pyogenic (bacterial) meningitis and acute aseptic (viral) meningitis, acute focal suppurative infections, including brain abscess, subdural empyema, and extradural abscess, chronic bacterial meningoencephalitis, including tuberculosis and mycobacterioses, neurosyphilis, and neuroborreliosis (Lyme disease), viral meningoencephalitis, including arthropod-borne (Arbo) viral encephalitis, Herpes simplex virus Type 1, Herpes simplex virus Type 2, Varicalla-zoster virus (Herpes zoster), cytornegalovirus, poliomyelitis, rabies, and human immunodeficiency virus 1, including FHV-I meningoencephalitis (subacute encephalitis), vacuolar myelopathy, AIDS-associated myopathy, peripheral neuropathy, and AIDS in children, progressive multifocal leukoencephalopathy, subacute sclerosing panencephalitis, fungal meningoencephalitis, other infectious diseases of the nervous system; transmissible spongiform encephalopathies (prion diseases); demyelinating diseases, including multiple sclerosis, multiple sclerosis variants, acute disseminated encephalomyelitis and acute necrotizing hemorrhagic encephalomyelitis, and other diseases with demyelination; degenerative diseases, such as degenerative diseases affecting the cerebral cortex, including Alzheimer disease and Pick disease, degenerative diseases of basal ganglia and brain stem, including Parkinsonism, idiopathic Parkinson disease paralysis agitans), progressive supranuclear palsy, corticobasal degeneration, multiple system atrophy, including striatonigral degenration, Shy-Drager syndrome, and olivopontocerebellar atrophy, and Huntington disease; spinocerebellar degenerations, including spinocerebellar ataxias, including Friedreich ataxia, and ataxia-telanglectasia, degenerative diseases affecting motor neurons, including amyotrophic lateral sclerosis (motor neuron disease), bulbospinal atrophy (Kennedy syndrome), and spinal muscular atrophy; inborn errors of metabolism, such as leukodystrophies, including Krabbe disease, metachromatic leukodystrophy, adrenoleukodystrophy, elizaeus-Merzbacher disease, and Canavan disease, mitochondrial encephalomyopathies, including Leigh disease and other mitochondrial encephalomyopathies; toxic and acquired metabolic diseases, including vitamin deficiencies such as thiamine (vitamin BI) deficiency and vitamin B12 deficiency, neurologic sequelae of metabolic disturbances, including hypoglycemia, hyperglycemia, and hepatic encephatopathy, toxic disorders, including carbon monoxide, methanol, ethanol, and radiation, including combined methotrexate and radiation-induced injury, tumors, such as gliomas, including astrocytoma, including fibrillary (diffuse) astrocytoma and glioblastoma multiforme, pilocytic astrocytoma, pleomorphic xanthoastrocytoma, and brain stem glioma, oligodendroglioma, and ependymoma and related paraventricular mass lesions, neuronal tumors, poorly differentiated neoplasms, including medulloblastoma, other parenchymal tumors, including primary brain lymphoma, germ cell tumors, and pineal parenchymal tumors, meningiomas, metastatic tumors, paraneoplastic syndromes, peripheral nerve sheath tumors, including schwannoma, neurofibroma, and malignant peripheral nerve sheath tumor (malignant schwannoma), and neurocutaneous syndromes (phakomatoses), including neurofibromotosis, including Type I neurofibromatosis (NFI) and TYPE 2 neurofibromatosis (NF2), tuberous sclerosis, and Von Hippel-Lindau disease.

Pharmacogenomics

Test/candidate compounds, or modulators which have a stimulatory or inhibitory effect on RGS9 or evectin polypeptide activity (e.g., RGS9 or evectin gene expression) as identified by a screening assay described herein can be administered to individuals to treat (prophylactically or therapeutically) disorders (e.g., neurological disorders) associated with aberrant RGS9 or evectin polypeptide activity. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permit the selection of effective compounds (e.g., drugs) for prophylactic or therapeutic treatments based on a consideration of the individual's genotype. Such pharmacogenomics can further be used to determine appropriate dosages and therapeutic regimens. Accordingly, the activity of RGS9 or evectin polypeptide, expression of RGS9 or evectin nucleic acid, or mutation content of RGS9 or evectin genes in an individual can be determined to thereby select appropriate compound(s) for therapeutic or prophylactic treatment of the individual.

Pharmacogenomics deal with clinically significant hereditary variations in the response to drugs due to altered drug disposition and abnormal action in affected persons. See, e.g., Eichelbaum, 1996 and Linder, 1997. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase deficiency (GOD) is a common inherited enzymopathy in which the main clinical complication is haemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2136 and CYP2C 19) has provided an explanation as to why some patients do not obtain the expected drug effects or show exaggerated drug response and serious toxicity after taking the standard and safe dose of a drug.

These polymorphisms are expressed in two phenotypes in the population, the extensive metabolizer (EM) and poor metabolizer (PM). The prevalence of PM is different among different populations. For example, the gene coding for CYP2136 is highly polymorphic and several mutations have been identified in PM, which all lead to the absence of functional CYP2D6. Poor metabolizers of CYP2136 and CYP2C 19 quite frequently experience exaggerated drug response and side effects when they receive standard doses.

If a metabolite is the active therapeutic moiety, PM show no therapeutic response, as demonstrated for the analgesic effect of codeine mediated by its CYP2136-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of RGS9 or evectin polypeptide, expression of RGS9 or evectin nucleic acid, or mutation content of RGS9 or evectin genes in an individual can be determined to thereby select appropriate agent(s) for therapeutic or prophylactic treatment of a subject. In addition, pharnacogenetic studies can be used to apply genotyping of polymorphic alleles encoding drug-metabolizing enzymes to the identification of a subject's drug responsiveness phenotype. This knowledge, when applied to dosing or drug selection, can avoid adverse reactions or therapeutic failure and thus enhance therapeutic or prophylactic efficiency when treating a subject with a RGS9 or evectin modulator, such as a modulator identified by one of the exemplary screening assays described herein.

Monitoring of Effects During Clinical Trials

Monitoring the influence of compounds (e.g., drugs) on the expression or activity of RGS9 or evectin polypeptide/gene can be applied not only in basic drug screening, but also in clinical trials. For example, the effectiveness of an agent determined by a screening assay, as described herein, to increase RGS9 or evectin gene expression, protein levels, or up-regulate RGS9 or evectin activity, can be monitored in clinical trials of subjects exhibiting decreased RGS9 or evectin gene expression, protein levels, or down-regulated RGS9 or evectin polypeptide activity. Alternatively, the effectiveness of an agent, determined by a screening assay, to decrease RGS9 or evectin gene expression, protein levels, or down-regulate RGS9 or evectin polypeptide activity, can be monitored in clinical trials of subjects exhibiting increased RGS9 or evectin gene expression, protein levels, or up-regulated RGS9 or evectin polypeptide activity. In such clinical trials, the expression or activity of a RGS9 or evectin polypeptide and, preferably, other genes which have been implicated in, for example, a nervous system related disorder can be used as a “read out” or markers of the ligand responsiveness of a particular cell.

For example, and not by way of limitation, genes, including a RGS9 or evectin gene, which are modulated in cells by treatment with a compound (e.g., drug or small molecule) which modulates RGS9 or evectin polypeptide/gene activity (e.g., identified in a screening assay as described herein) can be identified. Thus, to study the effect of compounds on CNS disorders, for example, in a clinical trial, cells can be isolated and RNA prepared and analyzed for the levels of expression of a RGS9 or evectin gene and other genes implicated in the disorder. The levels of gene expression (i.e., a gene expression pattern) can be quantified by Northern blot analysis or RT-PCR, as described herein, or alternatively by measuring the amount of protein produced, by one of the methods described herein, or by measuring the levels of activity of a RGS9 or evectin polypeptide or other genes. In this way, the gene expression pattern can serve as an marker, indicative of the physiological response of the cells to the compound. Accordingly, this response state may be determined before, and at various points during, treatment of the individual with the compound.

In a preferred embodiment, the present invention provides a method for monitoring the effectiveness of treatment of a subject with a compound (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the compound; (ii) detecting the level of expression of a RGS9 or evectin polypeptide, mRNA, or genomic DNA in the preadministration sample; (iii) obtaining one or more post-administration samples from the subject; (iv) detecting the level of expression or activity of the RGS9 or evectin polypeptide, mRNA, or genomic DNA in the post-administration samples; (v) comparing the level of expression or activity of the RGS9 or evectin polypeptide, mRNA, or genomic DNA in the pre-administration sample with the RGS9 or evectin polypeptide, mRNA, or genomic DNA in the post administration sample or samples; and (vi) altering the administration of the compound to the subject accordingly. For example, increased administration of the compound may be desirable to increase the expression or activity of a RGS9 or evectin polypeptide/gene to higher levels than detected, i.e., to increase the effectiveness of the agent.

Alternatively, decreased administration of the agent may be desirable to decrease expression or activity of RGS9 or evectin to lower levels than detected, i.e. to decrease the effectiveness of the compound.

Pharmaceutical Compositions

The RGS9 or evectin nucleic acid molecules, RGS9 or evectin polypeptides (particularly fragments of RGS9 or evectin), modulators of a RGS9 or evectin polypeptide, and anti-RGS9 or evectin antibodies (also referred to herein as “active compounds”) of the invention can be incorporated into pharmaceutical compositions suitable for administration to a subject, e.g., a human. Such compositions typically comprise the nucleic acid molecule, protein, modulator, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a RGS9 or evectin polypeptide or anti-RGS9 or evectin antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems.

Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811 which is incorporated herein by reference.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al., 1994). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system. The pharmaceutical compositions can be included in a container, pack, or dispenser together with instructions for administration.

### EXAMPLES

The following examples are carried out using standard techniques, which are well known and routine to those of skill in the art, except where otherwise described in detail. The following examples are presented for illustrative purpose, and should not be construed in any way limiting the scope of this invention.

### Example 1

### Identification of RGS9 Interacting Proteins

Yeast Two-Hybrid Assay

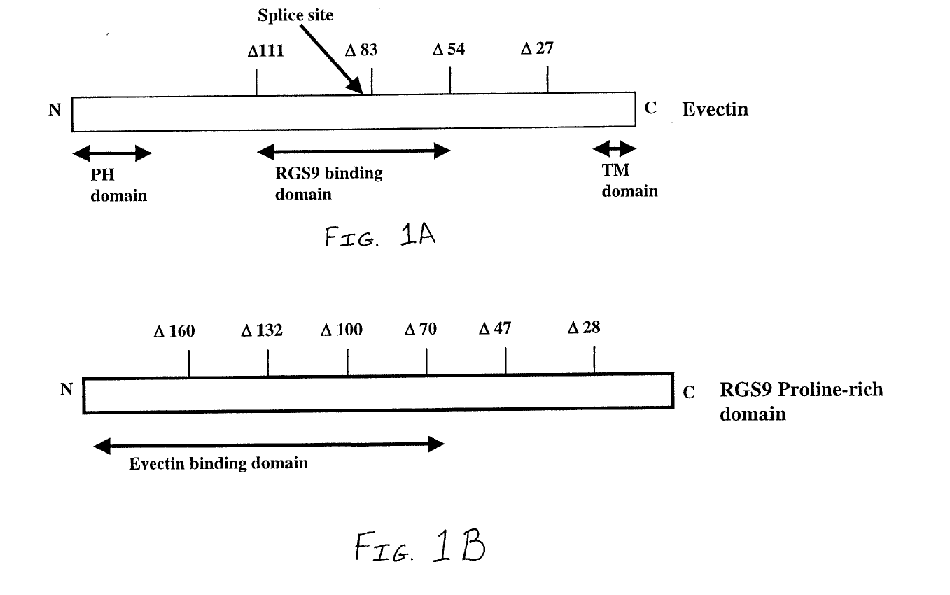

To identify interactors of the proline rich domain of RGS9, amino acids 460-672 of human RGS9 (FIG. 1A), was used as “bait” to screen a pretransformed human fetal brain library (Clontech). The RGS9-2 cDNA used was cloned by PCR from a human brain cDNA library using the primers 5′ primer: GCAAGCTTCCACCATGACAATCCGACACCAAGGCCAGCAG, 3′ primer: GCTCTAGATTACAGGCTCTCCCAGGGGCAGATGACC. The DNA encoding amino acids 460-672 of RGS9-2, was amplified by PCR using the primer sequences 5′ GGTCATATGACTGTGGACATCACCCAGCCGGGC and 3′ GGATCCTTACAGGCTCTCCCAGGGGCA and ligated into the NdeI and BamH 1 sites of pAS1 (Clontech). The pAS1-RGS9CT plasmid was used to transform the yeast strain PJ69-2A (Clontech) using lithium acetate. The library was screened using a yeast mating protocol by incubating the library (MATα) with the bait strain (MATα) overnight at 30° C. according to the manufacturer's protocols. The mating mixture was plated on SD/-His/-Leu/-Trp and incubated at 30° C. until colonies appeared. Colonies were picked and miniprep DNA prepared. The insert in the library plasmid was amplified using the LD-Insert Screening amplimer set of primers (Clontech) and the amplicon sequenced on an ABI 3700 capillary sequencer. The sequences obtained were used as queries against the GENBANK database using BLAST. The 6 most common hits were selected for further study and were replated on SD/-His/-Leu/-Trp containing 0-40 mM 3 aminotetrazole. The specificity of the protein-protein interactions were confirmed using unrelated baits (SNF, RAT Growth hormone receptor and N terminus of Kv4.3) and also unloaded DNA binding domain vector (pAS). Deletion mutants were made using PCR to introduce stop codons at specific points in the sequence and their ability to interact investigated using the yeast 2 hybrid assay.

Results

Using the amino acids 460-672 of human RGS9-2 (FIG. 1A), as bait, more than 1000 colonies were positive for histidine hypertrophy by plating on SD/-His/-Leu/-Trp. Miniprep DNA from 360 of these was prepared and sequenced. 207 high quality sequences were obtained and used to BLAST the genbank database. Of these, 22 were identified as a protein called evectin (Krappa et al 1999), also known in the literature as PHR1 (Xu et al 1999). This protein was of immediate interest because it contains an N-terminal pleckstrin homology domain, a protein motif commonly found in proteins of signal transduction pathways. Furthermore PHR1 has been reported to interact with G-protein βγ subunits. At least 2 alternatively spliced forms exist and interestingly only a short form, lacking exon 2 encoding 35 amino acids (FIG. 1B), interacts with RGS9. PCR analysis indicates that this shorter form of evectin is more highly and widely expressed than the longer form. Control experiments with unrelated baits indicate that in yeast, this interaction is specific, since evectin was shown not to interact with other baits such as SNF, the rat growth hormone receptor or the N terminus of the ion channel Kv4.3N, in the yeast 2 hybrid assay. However, in the yeast strain expressing just pAS-evectin and pACT (the empty DNA binding domain vector) limited growth was seen and this is consistent with other reports of minor constitutive activity when baits are co-expressed with empty vectors.

The clone discovered using the yeast two-hybrid assay corresponds to the latter half of the evectin protein (amino acids 101-190) suggesting that the interaction motif is in the C terminal region of evectin. To further investigate and to more accurately define the region of interaction between the RGS9-2, and evectin, a series of deletion mutants were made at the C-termini of both proteins. The ability of the mutants to interact was investigated using the yeast two-hybrid assay. This study identified a domain (amino acids 79-136, see FIG. 1B) in evectin which is required for the interaction. Interestingly, the longer form would have an additional 35 amino acids at position 113 of this domain which is consistent with the lack of interaction of the longer splice variant of evectin in the yeast assay. The evectin binding site in the RGS9 is found between amino acids (461-602) of the proline rich domain.

The observation that evectin binds RGS9-2, and its reported ability to bind G protein βγ subunits suggests that it may have important roles in the scaffolding of protein components of signal transduction cascades. This may allow these molecules to be bought together such that intracellular signaling is both specific and efficient. Since the different binding capabilities of evectin are mediated through distinct motifs/domains, it is contemplated to therapeutically modulate the RGS9-evectin interaction without affecting the ability of evectin to interact with G protein βγ dimers.

TABLE 3
Table of interactions of C terminal evectin
and RGS 9. The C terminal mutants were
prepared using PCR and their ability to
interact with RGS9 for evectin mutants or
evectin for the RGS9 mutants was investigated
using the yeast 2 hybrid assay. Also shown are
the results for the control assay using yeast
strains transformed with the empty pAS vector.

| | w/pAS | w/RGS9 |
|---|---|---|
| EVT ΔC27 | — | + |
| EVT ΔC54 | — | + |
| EVT ΔC83 | — | — |
| EVT ΔC111 | — | — |
| | w/pACT | w/EVT600 |
| RGS9 ΔC28 | — | — |
| RGS9 ΔC47 | — | + |
| RGS9 ΔC70 | — | + |
| RGS9 ΔC100 | — | +/− |
| RGS9 ΔC132 | — | — |
| RGS9 ΔC160 | — | +/− |

### Example 2

### Expression of Recombinant RGS9 and evectin Polypeptide in Bacterial Cells

In this example, RGS9 and/or evectin is expressed as a recombinant glutathione-S-transferase (GST) fusion polypeptide in E. coli and the fusion polypeptide is isolated and characterized. Specifically, RGS9 and/or evectin is fused to GST and this fusion polypeptide is expressed in E. coli, e.g., strain PEB 199. As the human polypeptide of SEQ ID NO:2 and SEQ ID NO:4, are predicted to be approximately 77 kDa and 21.2 kDa, respectively; and GST is predicted to be 26 kDa, the fusion protein is predicted to be approximately 103 kDa and 47.2 kDa, in molecular weight, respectively. Expression of the GST-RGS9 and/or evectin fusion polypeptide in PEB199 is induced with IPTG. The recombinant fusion polypeptide is purified from crude bacterial lysates of the induced PEB 199 strain by affinity chromatography on glutathione beads. Using polyacrylamide gel electrophoretic analysis of the polypeptide purified from the bacterial lysates, the molecular weight of the resultant fusion protein is determined.

### Example 3

### Expression of Recombinant RGS9 and Evectin Polypeptide in COS Cells

To express the RGS9 and/or evectin in COS cells, the pcDNA/Amp vector by Invitrogen Corporation (San Diego, Calif.) will be used. This vector contains an SV40 origin of replication, an ampicillin resistance gene, an E. coli replication origin, a CMV promoter followed by a polylinker region, and an SV40 intron and polyadenylation site. A DNA fragment encoding the entire RGS9 and evectin protein and a HA tag (Wilson et al., 1984) fused in-frame to its 3′ end of the fragment is cloned into the polylinker region of the vector, thereby placing the expression of the recombinant protein under the control of the CMV promoter.

To construct the plasmid, the RGS9 and/or evectin DNA sequence is amplified by PCR using two primers. The 5′ primer contains the restriction site of interest followed by approximately twenty nucleotides of the RGS9 and/or evectin coding sequence starting from the initiation codon; the 3′ end sequence contains complementary sequences to the other restriction site of interest, a translation stop codon, the HA tag and the last 20 nucleotides of the RGS9 and/or evectin coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the RGS9 and/or evectin gene is inserted in the correct orientation. The ligation mixture is transformed into E. coli cells (strains HB101, DH5a, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the RGS9 and/or evectin-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook et al., “Molecular Cloning: A Laboratory Manual,” 2nd, ed, Cold Spring Harbor Laboratory, Cold Spring Harbor Laboratory Press, Cold Spring Harbor, N.Y., 1989. The expression of the RGS9 and/or evectin polypeptide is detected by radiolabelling (³⁵S-methionine or ³⁵S-cysteine available from NEN, Boston, Mass., can be used) and immunoprecipitation (Harlow and Lane, “Antibodies: A Laboratory Manual,” Cold Spring Harbor Laboratory Press, Cold Spring Harbor, N.Y., 1988) using an HA specific monoclonal antibody. Briefly, the cells are labelled for 8 hours with ³⁵S-methionine (or ³⁵S-cysteine). The culture media are then collected and the cells are lysed using detergents (RIPA buffer, 150 mM NaCl, 1%NP-40, 0.1% SDS, 0.5% DOC, 50 mM Tris, pH 7.5). Both the cell lysate and the culture media are precipitated with an HA specific monoclonal antibody. Precipitated proteins are then analyzed by SDS-PAGE.

Alternatively, DNA containing the RGS9 and/or evectin coding sequence is cloned directly into the polylinker of the pCDNA/Amp vector using the appropriate restriction sites. The resulting plasmid is transfected into COS cells in the manner described above, and the expression of the RGS9 and/or evectin polypeptide is detected by radiolabelling and immunoprecipitation using an RGS9 and/or evectin specific monoclonal antibody.

### Example 4

### Cell Line Generation

This example describes how one would generate a cell line comprising the open reading frame polynucleotide sequence of SEQ ID NO:1 or SEQ ID NO:3. The RGS9 and/or evectin polynucleotide sequence of SEQ ID NO:1 or SEQ ID NO:3 is ligated into the mammalian expression vector pCDNA3.1+zeo (Invitrogen, 1600 Faraday Avenue, Carlsbad, Calif. 92008). HEK 293 cells stabley expressing a suitable G-protein coupled receptor are transfected with the plasmid and selected with 500 ug/ml zeocin. Zeocin resistant clones are tested for expression of RGS9 and/or evectin by RT-PCR and western blotting. Subsequently, the effects of RGS9 and/or evectin expression on receptor signaling is investigated.

### Example 5

### Construction of RGS9 and Evectin Gene Targeting Vector

A partial murine cDNA clone can be isolated from a mouse macrophage cDNA library (e.g., obtained commercially from Stratagene) using the full length human RGS9 and/or evectin coding sequence as a probe by standard techniques. The murine RGS9 and/or evectin cDNA is then used as a probe to screen a genomic DNA library made from the 129 SJ strain of mouse, again using standard techniques. The isolated murine RGS9 and/or evectin genomic clones are then subcloned into a plasmid vector, pBluescript (obtained commercially from Stratagene), for restriction mapping, partial DNA sequencing, and construction of the targeting vector. To functionally disrupt the RGS9 and/or evectin gene, a targeting vector would be prepared in which non-homologous DNA is inserted within a selected exon sequence, deleting a portion of RGS9 and/or evectin coding sequence in the process and rendering the remaining downstream RGS9 and/or evectin coding sequences out of frame with respect to the start of translation. The RGS9 and/or evectin targeting vector is constructed using the plasmid RGS9 and/or evectin. This plasmid will carry the neomycin phosphotransferase (neo) gene under the control of the phosphoglycerokinase promoter and the herpes simplex thymidine kinase (HSV tk) gene under the control of the same promoter. The positive selection neo gene is located within exon the selected exon sequence and in the same orientation as the gene, whereas the negative selection HSV tk gene is at the 3′ end of the construct. This configuration allows for the use of the positive and negative selection approach for homologous recombination (Mansour et al., 1988). Prior to transfection into embryonal stem cells, the plasmid is linearized by digestion.

### Example 6

### Transfection and Analysis Of Embryonal Stem Cells

D3 embryonal stem cells (Doestschman, 1985) are cultured on a neomycin resistant embryonal fibroblast feeder layer grown in Dulbecco's Modified Eagles medium supplemented with 15% Fetal Calf Serum, 2 mM glutamine, penicillin (50 u/ml)/streptomycin (50 u/ml), non-essential amino acids, 100 uM 2-mercaptoethanol and 500 u/ml leukemia inhibitory factor. Medium is changed daily and D3 cells are subcultured every three days and are then transfected with linearized plasmid by electroporation (25 uF capacitance and 400 Volts). The transfected cells are cultured for the first 5 days in gancyclovir and neomycin and for the last 3 days in neomycin alone. After expanding the clones, an aliquot of cells is frozen in liquid nitrogen. DNA is prepared from the remainder of cells for genomic DNA analysis to identify clones in which homologous recombination had occurred between the endogenous RGS9 and/or evectin gene and the targeting construct. To prepare genomic DNA, ES cell clones are lysed in 100 mM Tris-HCl, pH 8.5, 5 mM EDTA, 0.2% SDS, 200 mM NaCl and 100 ug of proteinase K/ml. DNA is recovered by isopropanol precipitation, solubilized in 10 mM Tris-HCl, pH 8.0/0.1 mM EDTA. To identify homologous recombinant clones, genomic DNA isolated from the clones is digested with restriction enzymes. After restriction digestion, the DNA can be resolved on a 0.8% agarose gel, blotted onto a Hybond-N membrane and hybridized at 65° C., with probes that bind a region of the RGS9 and/or evectin gene proximal to the 5′ end of the targeting vector and probes that bind a region of the RGS9 and/or evectin gene distal to the 3′ end of the targeting vector. The locations of the two probes within the mouse RGS9 and/or evectin gene are illustrated in FIG. Q. After standard hybridization, the blots are washed with 40 mM NaPO4 (pH 7.2), 1 mM EDTA and 1% SDS at 65° C., and exposed to X-ray film. Hybridization of the 5′ probe to the wild type RGS9 and/or evectin allele results in a fragment readily discernible by autoradiography from the mutant RGS9 and/or evectin allele having the neo insertion.

### Example 7

### Generation of RGS9 and/or Evectin Deficient Mice

Female and male mice are mated and blastocysts are isolated at 3.5 days of gestation. 10 to 12 cells from the clone described in Example 5 are injected per blastocyst and 7 or 8 blastocysts are implanted in the uterus of a pseudopregnant female. Pups are delivered by cesarean section on the 18th day of gestation and placed with a foster BALB/c mother. Male and female chimeras are mated with female and male C57/B6 mice, respectively, and germline transmission is determined by the agouti coat color. Mendelian genetics predicts that 50% of the offspring with agouti coat color derived from mating chimeras with C57/B6 mice will be heterozygous for the RGS9 and/or evectin null mutation. These heterozygous animals are mated and, again Mendelian genetics predicts that approximately 25% of the offspring will be homozygous for the RGS9 and/or evectin null mutation. Genotyping of the animals is accomplished by obtaining tail genomic DNA and analysing as described for the ES cells in Example 5.

To confirm that the RGS9 and/or evectin −/− mice do not express full-length RGS9 and/or evectin mRNA transcripts, RNA is isolated from various tissues and analyzed by standard Northern hybridizations with an RGS9 and/or evectin cDNA probe or by reverse transcriptase-polymerase chain reaction (RT-PCR). RNA is extracted from various organs of the mice using 4M Guanidinium thiocyanate followed by centrifugation through 5.7 M CsCl as described in Sambrook et al. (Molecular Cloning: A Laboratory Manual, 2nd Edition, Cold Spring Harbor Laboratory press, 1989). Primers specific for the neomycin gene will detect a transcript in RGS9 and/or evectin +/− and −/− but not +/+ animals. Northern and RT-PCT analyses are used to confirm that homozygous disruption of the RGS9 and/or evectin gene results in the absence of detectable full-length RGS9 and/or evectin mRNA transcripts in the RGS9 and/or evectin −/− mice.

To examine RGS9 and/or evectin protein expression in the RGS9 and/or evectin deficient mice, Western blot analyses are performed on macrophage cell lysates. 8 week old mice are injected i.p. with thioglycollate medium (commercially obtained from Sigma Chemical Co., St. Louis, Mo.). Peritoneal exudate cells (PECs) are harvested 4-5 days later. Macrophages are purified from the PECs by adherence to plastic in RPMI 1640 without serum for 2 hr at 37 C. Macrophage cell lysates are separated on 10% SDS-polyacrylamide gels, then transferred to nitrocellulose filters (commercially obtained from Amersham). Filters are probed with a rabbit antibody to human RGS9 and/or evectin protein. Detection is carried out using a secondary, horse radish peroxidase-linked, anti-rabbit antibody (from Amersham) and the Amersham ECL system according to the manufacturer's instructions. These results will confirm that homozygous disruption of the RGS9 and/or evectin gene results in an absence of detectable RGS9 and/or evectin protein in the −/− mice.

### Example 8

### Inhibition of RGS9 and/or Evectin Production

Design of RNA Molecules as Compositions of the Invention

All RNA molecules in this experiment are approximately 600 nts in length, and all RNA molecules are designed to be incapable of producing functional RGS9 and/or evectin protein. The molecules have no cap and no poly-A sequence; the native initiation codon is not present, and the RNA does not encode the full-length product. The following RNA molecules are designed:

(1) a single-stranded (ss) sense RNA polynucleotide sequence homologous to a portion of RGS9 and/or evectin murine messenger RNA (mRNA);

(2) a ss anti-sense RNA polynucleotide sequence complementary to a portion of RGS9 and/or evectin murine mRNA,

(3) a double-stranded (ds) RNA molecule comprised of both sense and anti-sense a portion of RGS9 and/or evectin murine mRNA polynucleotide sequences,

(4) a ss sense RNA polynucleotide sequence homologous to a portion of RGS9 and/or evectin murine heterogeneous RNA (hnRNA),

(5) a ss anti-sense RNA polynucleotide sequence complementary to a portion of RGS9 and/or evectin murine hnRNA,

(6) a ds RNA molecule comprised of the sense and anti-sense RGS9 and/or evectin murine hnRNA polynucleotide sequences,

(7) a ss murine RNA polynucleotide sequence homologous to the top strand of the a portion of RGS9 and/or evectin promoter,

(8) a ss murine RNA polynucleotide sequence homologous to the bottom strand of the a portion of RGS9 and/or evectin promoter, and

(9) a ds RNA molecule comprised of murine RNA polynucleotide sequences homologous to the top and bottom strands of the RGS9 and/or evectin promoter.

The various RNA molecules of (1)-(9) above may be generated through T7 RNA polymerase transcription of PCR products bearing a T7 promoter at one end. In the instance where a sense RNA is desired, a T7 promoter is located at the 5′ end of the forward PCR primer. In the instance where an antisense RNA is desired, the T7 promoter is located at the 5′ end of the reverse PCR primer. When dsRNA is desired both types of PCR products may be included in the T7 transcription reaction. Alternatively, sense and anti-sense RNA may be mixed together after transcription.

Construction of Expression Plasmid Encoding a Fold-Back Type of RNA

Expression plasmid encoding an inverted repeat of a portion of the RGS9 and/or evectin gene may be constructed using the information disclosed in this application. Two RGS9 and/or evectin gene fragments of approximately at least 600 nucleotides in length, almost identical in sequence to each other, may be prepared by PCR amplification and introduced into suitable restriction of a vector which includes the elements required for transcription of the RGS9 and/or evectin fragment in an opposite orientation. CHO cells transfected with the construct will produce only fold-back RNA in which complementary target gene sequences form a double helix. The genomic and PCR primer coordinates are based on the sequence of SEQ ID NO:1 or SEQ ID NO:3.

Assay

Balb/c mice (5 mice/group) may be injected intramuscularly or intraperitoneally with the murine RGS9 and/or evectin chain specific RNAs described above or with controls at doses ranging between 10 μg and 500 μg. Sera is collected from the mice every four days for a period of three weeks and assayed for RGS9 and/or evectin levels using the antibodies as disclosed herein.

### Example 9

### Method of the Invention in the Prophylaxis of Disease In Vivo Assay

Using the RGS9 and/or evectin specific RNA molecules described in Example 8, which do not have the ability to make RGS9 and/or evectin protein and RGS9 and/or evectin specific RNA molecules as controls, mice may be evaluated for protection from RGS9 and/or evectin related disease through the use of the injected RGS9 and/or evectin specific RNA molecules of the invention. Balb/c mice (5 mice/group) may be immunized by intercranial injection with the described RNA molecules at doses ranging between 10 and 500 μg RNA. At days 1, 2, 4 and 7 following RNA injection, the mice may be observed for signs of RGS9 and/or evectin related phenotypic change.

According to the present invention, because the mice that receive dsRNA molecules of the present invention which contain the RGS9 and/or evectin sequence may be shown to be protected against RGS9 and/or evectin related disease. The mice receiving the control RNA molecules may not be protected. Mice receiving the ss RNA molecules which contain the RGS9 and/or evectin sequence may be expected to be minimally, if at all, protected, unless these molecules have the ability to become at least partially double stranded in vivo.

According to this invention, because the dsRNA molecules of the invention do not have the ability to make RGS9 and/or evectin protein, the protection provided by delivery of the RNA molecules to the animal is due to a non-immune mediated mechanism that is gene specific.

### Example 10

### RNA Interference in Drosophila and Chinese Hamster Cultured Cells

To observe the effects of RNA interference, either cell lines naturally expressing RGS9 and/or evectin can be identified and used or cell lines which express RGS9 and/or evectin as a transgene can be constructed by well known methods (and as outlined herein). As examples, the use of Drosophila and CHO cells are described. Drosophila S2 cells and Chinese hamster CHO-K1 cells, respectively, may be cultured in Schneider medium (Gibco BRL) at 25° C. and in Dulbecco's modified Eagle's medium (Gibco BRL) at 37° C. Both media may be supplemented with 10% heat-inactivated fetal bovine serum (Mitsubishi Kasei) and antibiotics (10 units/ml of penicillin (Meiji) and 50 μg/ml of streptomycin (Meiji)).

Transfection and RNAi Activity Assay

S2 and CHO-K1 cells, respectively, are inoculated at 1×10⁶ and 3×10⁵ cells/ml in each well of 24-well plate. After 1 day, using the calcium phosphate precipitation method, cells are transfected with RGS9 and/or evectin dsRNA (80 pg to 3 μg). Cells may be harvested 20 h after transfection and RGS9 and/or evectin gene expression measured.

### Example 11

### Antisense Inhibition In Vertebrate Cell Lines

Antisense can be performed using standard techniques including the use of kits such as those of Sequitur Inc. (Natick, Mass.). The following procedure utilizes phosphorothioate oligodeoxynucleotides and cationic lipids. The oligomers are selected to be complementary to the 5′ end of the mRNA so that the translation start site is encompassed.

1) Prior to plating the cells, the walls of the plate are gelatin coated to promote adhesion by incubating 0.2% sterile filtered gelatin for 30 minutes and then washing once with PBS. Cells are grown to 40-80% confluence. Hela cells can be used as a positive control.

2) the cells are washed with serum free media (such as Opti-MEMA from Gibco-BRL).

3) Suitable cationic lipids (such as Oligofectibn A from Sequitur, Inc.) are mixed and added to serum free media without antibiotics in a polystyrene tube. The concentration of the lipids can be varied depending on their source. Add oligomers to the tubes containing serum free media/cationic lipids to a final concentration of approximately 200 nM (50-400 nM range) from a 100 μM stock (2 μl per ml) and mix by inverting.

4) The oligomer/media/cationic lipid solution is added to the cells (approximately 0.5 mls for each well of a 24 well plate) and incubated at 37° C. for 4 hours.

5) The cells are gently washed with media and complete growth media is added. The cells are grown for 24 hours. A certain percentage of the cells may lift off the plate or become lysed. Cells are harvested and RGS9 and/or evectin gene expression is measured.

### Example 12

### Identification of RGS9 and/or Evectin Binding Proteins and Agonists/Antagonists

Yeast strains, bacterial strains and media for yeast and bacterial selections and growth are well known in the art (see e.g., Klein et al., 1989(a), 1989b; Bartel et al., 1993(b)), as are plating procedures (Rose et al., 1990). A RGS9 and/or evectin polypeptide of the invention is expressed as a fusion protein (‘bait’) in the binding domain portion of the GAL4 protein in the pAS2-1 vector. A human brain library is then expressed in the form of fusions (prey) to the activation domain portion of the GAL4 protein in the pACT II vector. Functional interaction of RGS9 and/or evectin with a library protein will drive the expression of the reporter gene activity. The reporter phenotypes to be utilized are histidine prototrophy and beta-galactosidase activity. The ______ used as bait will be the human cDNA from the start codon to stop codon of SEQ ID NO:1 or SEQ ID NO:3. Protein interactions identified as described above, may further be screened with ligands, wherein the ligand may attenuate the protein-protein interaction, or alternatively, the ligand may induce a protein-protein interaction, not detected in the absence of the ligand.

### Example 13

### Assays

Cells expressing receptor, RGS 9 and evectin, produced as in example 4, can be used to screen for compounds which increase (agonists) or decrease (antagonists) the effects of the RGS9-evectin polypeptide dimer. The effects of test compound can be screened in functional assays in which the dimer modulates the signaling of a G protein coupled receptor which can be detected by binding assays, ligand binding assays, cAMP assays, inositol phosphate determination assays, functional assays in Xenopus Oocytes or microphysiometric assays.

Inositol Phosphate Assays

Cells expressing receptors which couple to inositol phosphate production via the Gq G α subunit are cultured overnight in the presence of [³H]-myo D-inositol. Cells are washed in cell culture media and then incubated at 37° C. in media containing 10 mM LiCl, which inhibits the inositol monophosphatase enzyme resulting in an accumulation of inositol monophosphate when the formation of inositol phoshates is stimulated by receptor activation. The cells are then stimulated with agonist in the presence of LiCl for 30 minutes. The assay is terminated with 5% percholoric acid. Levels of inositol phosphates are determined by ion exchange chromatography and liquid scintillation counting.

Cyclase Assays

4×10⁵ cells are plated into 96 well Biocoat cell culture plates (Becton Dickinson, 1 Becton Drive, Franklin Lakes, N.J. 07417-1886) 24 hours prior to assay. The cells are then incubated in Krebs-bicarbonate buffer at 37° C. for 15 minutes. A 5 minute pretreatment with 500 uM isobutylmethyl xanthine (IBMX) precedes a 12 minute stimulation with varying concentrations of dopamine in the presence of 1 uM forskolin. cAMP levels are determined using the SPA assay (Amersham Pharmacia Biotech, 800 Centennial Avenue, Pistcataway, N.J. 08855).

Ligand Binding Assays

Ligand binding assays provide a direct method for ascertaining receptor pharmacology and are adaptable to a high throughput format. The purified ligand for a receptor is radiolabeled to high specific activity (50-2000 Ci/mmol) for binding studies. A determination is then made that the process of radiolabeling does not diminish the activity of the ligand towards its receptor. Assay conditions for buffers, ions, pH and other modulators such as nucleotides are optimized to establish a workable signal to noise ratio for both membrane and whole cell receptor sources. For these assays, specific receptor binding is defined as total associated radioactivity minus the radioactivity measured in the presence of an excess of unlabeled competing ligand. Where possible, more than one competing ligand is used to define residual nonspecific binding. The effects of RGS9 and/or evectin on the binding kinetics can further be determined.

### Example 14

### Functional Assay in Xenopus Oocytes

Capped RNA transcripts from linearized plasmid templates encoding a receptor cDNA and cDNAs of RGS9 and/or evectin are synthesized in vitro with RNA polymerases in accordance with standard procedures. In vitro transcripts are suspended in water at a final concentration of 0.2 mg/ml. Ovarian lobes are removed from adult female toads, Stage V defolliculated oocytes are obtained, and RNA transcripts (10 ng/oocyte) are injected in a 50 nl bolus using a microinjection apparatus. Two electrode voltage clamps are used to measure the currents from individual Xenopus oocytes in response to agonist exposure. Recordings are made in Ca2+ free Barth's medium at room temperature. The Xenopus system can be used to screen known ligands and tissue/cell extracts for activating ligands.

### Example 15

### Microphysiometric Assays

Activation of a wide variety of secondary messenger systems results in extrusion of small amounts of acid from a cell. The acid formed is largely as a result of the increased metabolic activity required to fuel the intracellular signaling process. The pH changes in the media surrounding the cell are very small but are detectable by the CYTOSENSOR microphysiometer (Molecular Devices Ltd., Menlo Park, Calif.). The CYTOSENSOR is thus capable of detecting the activation of a receptor which is coupled to an energy utilizing intracellular signaling pathway and effects of modulating proteins such as RGS9 and evectin can be determined.

### Example 16

### Calcium Functional Assays

Receptors coupled to Gq when expressed in HEK 293 cells have been shown to be coupled functionally to activation of PLC and calcium mobilization. HEK 293 cells expressing recombinant receptors are then loaded with fura 2, determination of basal calcium levels in the HEK 293 cells in receptor-transfected or vector control cells should be observed, wherein the normal concentration range is 100 nM to 200 nM and selected ligands or tissue/cell extracts are evaluated for agonist induced calcium mobilization and the effects of RGS9 and/or evectin determined.

Equivalents: Those skilled in the art will recognize, or be able to ascertain using no more than routine experimentation, many equivalents to the specific embodiments of the invention described herein. Such equivalents are intended to be encompassed by the following claims.

### REFERENCES

International Application No. EP A02323621

International Application No. EP 0036776

International Application No. EP 0859055

International Application No. EP 125,023

International Application No. EP 171,496

International Application No. EP 171,496

International Application No. EP 184,187

International Application No. EP 264166

International Application No. PCT/US86/02269

U.S. Pat. No. 4,196,265

U.S. Pat. No. 4,522,811

U.S. Pat. No. 4,554,101

U.S. Pat. No. 4,683,195

U.S. Pat. No. 4,683,202

U.S. Pat. No. 4,736,866

U.S. Pat. No. 4,816,567

U.S. Pat. No. 4,870,009

U.S. Pat. No. 4,873,191

U.S. Pat. No. 4,873,316

U.S. Pat. No. 4,987,071

U.S. Pat. No. 5,116,742

U.S. Pat. No. 5,223,409

U.S. Pat. No. 5,272,057

U.S. Pat. No. 5,283,317

U.S. Pat. No. 5,328,470

U.S. Pat. No. 5,498,531

U.S. Pat. No. 5,766,844

U.S. Pat. No. 5,789,654

U.S. Pat. No. 5,798,209

U.S. Pat. No. 6,201,103

U.S. SIR No. H1,892

International Application No. WO 86/01533

International Application No. WO 90/02809

International Application No. WO 90/11354

International Application No. WO 91/01140

International Application No. WO 91/17271

International Application No. WO 92/01047

International Application No. WO 92/0968

International Application No. WO 92/09690

International Application No. WO 92/15679

International Application No. WO 92/18619

International Application No. WO 92/20791

International Application No. WO 93/01288

International Application No. WO 93/04169

International Application No. WO 94/10300

International Application No. WO 94/1610 1

International Application No. WO 97/07668

International Application No. WO 97/07669

International Application No. WO 00/63364

Abravaya et al., Nucleic Acids Res., 23:675-682, 1995.

Adams et al., Nature 355:632-634, 1992.

Adams et al., Nature 377 Supp:3-174, 1995.

Adams et al., Science 252:1651-1656, 1991.

Altschul et al., J. Molec. Biol. 215:403-410, 1990.

Amann et al., Gene 69:301-315, 1988.

Baldari et al., Embo J. 6:229-234, 1987.

Banerji et al., Cell, 33:729-740; 1983.

Bartel and Szostak, Science 261:1411-1418, 1993.

Bartel et al. Biotechniques 14:920-924, 1993(b).

Bartel, “Cellular Interactions and Development: A Practical Approach”, pp. 153-179, 1993(a).

Bradley, Current Opinion in Biotechnology 2:823-829, 1991.

Bradley, in “Teratocarcinomas and Embryonic Stem Cells: A Practical Approach,” E. J. Robertson, ed., IRL, Oxford, pp. 113-152, 1987.

Bunzow et al., Nature, 336:783-787, 1988.

Burge and Karlin, “Prediction of complete gene structures in human genomic DNA.” J. Mol. Biol. 268:78-94, 1997.

Byrne and Ruddle, PNAS 86:5473-5477, 1989.

Calame and Eaton, Adv. Immunol. 43:235-275, 1988.

Campes and Tilghman, Genes Dev. 3:537-546, 1989.

Chen et al., PNAS 91:3054-3057, 1994.

Cohen et al., Adv. Chromatogr. 36:127-162, 1996.

Cotton et al., PNAS 85:4397, 1988.

Cotton, Mutat. Res. 285:125-144, 1993.

Cowan et al., “RGS Proteins: Lessons from the RGS9 subfamily,” Progress in Nucleic Acid Research and Molecular Biology 65:341-359, 2001.

D'Eustachio et al., Science 220:919-924, 1983.

Devereux et al., Nucleic Acids Research 12(1):387, 1984.

Doestschman et al., J. Embryol. Exp. Morphol. 87:27-45, 1985.

Edlund et al., Science 230:912-916, 1985.

Eichelbaum, Clin. Exp. Pharmacol Physiol, 23(10-11):983-985, 1996.

Elledge et al., Proc. Natl. Acad. Sci. USA, 88:1731-1735, 1991.

Fan, Y. et al., PNAS, 87:6223-27, 1990.

Finely et al., Proc. Natl. Acad. Sci. USA, 91:12980-12984, 1994.

Frohman et al, Proc. Natl. Acad. Sci. USA 85, 8998-9002, 1988.

Gaultier et al., Nucleic Acids Res. 15:6625-6641, 1987.

Gentz et al., Proc. Natl. Acad. Sci. USA, 86:821-824, 1989.

Griffin et al., Appl. Biochem. Biotechnol. 38:147-159, 1993.

Gunnar von Heijne, “Membrane Protein Structure Prediction, Hydrophobicity Analysis and the Positive-inside Rule” J. Mol. Biol., 225:487-494, 1992.

Harlow and Lane, “Antibodies: A Laboratory Manual,” Cold Spring Harbor Laboratory Press, Cold Spring Harbor, N.Y., 1988

Harper et al., Cell, 75:805-816, 1993.

Haselhoff and Gerlach, Nature 334:585-591, 1988.

Hayashi, Genet. Anal. Tech. Appl. 9:73-79, 1992.

Helene et al., Ann. N.Y Acad Sci. 660:27-36, 1992.

Helene, Anticancer Drug Des. 6(6):569-84, 1991.

Hepler, “Emerging roles for RGS proteins in cell signalling,” Trends in Phamacological Sciences 20:376-382, 1999.

Hogan, “Manipulating the Mouse Embryo,” Cold Spring Harbor Laboratory Press, Cold Spring Harbor, N.Y., 1986.

Inoue et al., FEBS Lett. 215:327-330, 1987(a).

Inoue et al., Nucleic Acids Res. 15:6131-6148, 1987(b).

Iwabuchi et al., Oncogene 8:1693-1696, 1993.

Johnson et al., Endoc. Rev., 10:317-331, 1989.

Kaufman et al., EMBO J 6:187-195, 1987.

Kessel and Gruss, Science 249:3 74-3 79, 1990.

Klein et al., Curr. Genet., 16:145-152, 1989(b).

Klein et al., Curr. Genet. 13:29-35, 1989(a).

Krappa et al., “Evectins: Vesicular proteins that carry a pleckstrin homology domain and localize to post-Golgi membranes,” Proceedings of the National Academy of Sciences 96:4633-4368, 1999.

Kurj an and Herskowitz, Cell 933-943, 1982.

Kyte and Doolittle, J. Mol. Biol., 157:105-132, 1982.

Lakso et al., PJVAS 89:6232-6236, 1992.

Lefkowitz, Nature, 351:353-354, 1991.

Li et al., Cell 69:915, 1992.

Linder, Clin. Chem. 43(2):254-266, 1997.

Lucklow and Summers, Virology 170:31-39, 1989.

Madura et al., J. Biol. Chem. 268:12046-1205, 1993

Maher, Bioassays 14(12):807-15, 1992.

Mansour et al., Nature 336:348, 1988

Maxim and Gilbert, PNAS 74:560, 1977.

Morin et al., Nucleic Acids Res., 21:2157-2163, 1993.

Myers et al., Nature 313:495, 1985(a).

Myers et al., Science 230:1242, 1985(b).

O'Gon-nan et al., Science 251:1351-1355, 1991.

Orita et al., PNAS 86:2766, 1989.

Pinkert et al. Genes Dev. 1:268-277, 1987.

Queen and Baltimore, Cell 33:741-748, 1983.

Rahman et al., Journal of Neuroscience 19:2016-2026, 1999.

Rose et al, “Methods in Yeast Genetics: A Laboratory Course Manual.” Cold Spring Harbor Press, Cold Spring Harbor, N.Y. (1990).

Ross and Wilkie, “GTPase-activating proteins for Heterotrimeric G proteins: Regulators of G protein Signaling (RGS) and RGS-like proteins,” Annual Reiew of Biochemistry 69:795-827, 2000.

Saleeba et al., Meth. Enzymol. 217:286-295, 1992.

Sambrook et al., “Molecular Cloning: A Laboratory Manual” 2nd, ed, Cold Spring Harbor Laboratory, Cold Spring Harbor Laboratory Press, Cold Spring Harbor, N.Y., 1989.

Sanger, PNAS 74:5463, 1977.

Schultz et al., Gene 54:113-123, 1987.

Seed, Nature 329:840, 1987.

Simon et al., Science, 252:802-8, 1991.

Smith and Johnson, Gene 67:31-40, 1988.

Smith et al., Mol. Cell Biol. 3:2156-2165, 1983.

Songyang, et al., Cell 72:767-778, 1993

Studier et al. “Gene Expression Technology” Methods in Enzymology 185, 60-89, 1990.

Thomas and Capecchi, Cell 51:503, 1987.

Wilmut et al., Nature 385:810-813, 1997.

Wilson et al., Cell 37:767, 1984.

Winoto and Baltimore. EMBO J 8:729-733, 1989.

Xu et al., “PHR1 encodes an abundant, pleckstrin homology domain-containing Integral membrane protein in the photoreceptor outer segments,” Journal of Biological Chemistry 274:35676-35685, 1999.

Zervos et al., Cell 72:223-232, 1993.

What is claimed is:

1. An isolated human RGS9 polypeptide fragment comprising an evectin polypeptide binding domain, wherein the RGS9 polypeptide fragment comprises the amino acid sequence from amino acid 461 through amino acid 602 of SEQ ID NO:2.

2. An isolated human evectin polypeptide fragment comprising a RGS9 polypeptide binding domain, wherein the evectin polypeptide fragment comprises the amino acid sequence from amino acid 79 through amino acid 136 of SEQ ID NO:4.

3. An isolated polynucleotide encoding the RGS9 polypeptide fragment comprising the evectin binding domain of claim 1, wherein the polynucleotide comprises the nucleotide sequence of SEQ ID NO:1.

4. An isolated polynucleotide encoding the evectin polypeptide fragment comprising the RGS9 binding domain of claim 2, wherein the polynucleotide comprises the nucleotide sequence of SEQ ID NO:3.

5. An isolated polypeptide dimer comprising a RGS9 polypeptide and an evectin polypeptide.

6. The dimer of claim 5, wherein the RGS9 polypeptide comprises the amino acid sequence of SEQ ID NO:2 and the evectin polypeptide comprises the amino acid sequence of SEQ ID NO:4.

7. The dimer of claim 6, wherein the RGS9 polypeptide is encoded by a polynucleotide comprising the nucleotide sequence of SEQ ID NO:1 and the evectin polypeptide is encoded by a polynucleotide comprising the nucleotide sequence of SEQ ID NO:3.

8. An antibody specific for the RGS9-evectin dimer of claim 6.

9. An antibody specific for the RGS9 polypeptide fragment of claim 1.

10. An antibody specific for the evectin polypeptide fragment of claim 2.

11. A transgenic animal whose genome comprises an engineered functional disruption in the polynucleotide encoding the endogenous RGS9 polypeptide, wherein the disruption occurs in the evectin binding domain of the RGS9 polypeptide.

12. The transgenic animal of claim 11, wherein the animal is homozygous for the functional disruption.

13. A transgenic animal whose genome comprises an engineered functional disruption in the polynucleotide encoding the endogenous evectin polypeptide, wherein the disruption occurs in the RGS9 binding domain of the evectin polypeptide.

14. The transgenic animal of claim 13, wherein the animal is homozygous for the functional disruption.

15. A recombinant expression vector comprising a polynucleotide encoding the polypeptide fragment of claim 1.

16. A recombinant expression vector comprising a polynucleotide encoding the polypeptide fragment of claim 2.

17. A recombinant expression vector comprising a polynucleotide encoding the polypeptide dimer of claim 5.

18. A genetically engineered host cell, transfected, transformed or infected with the vector according to claims 15, 16 or 17.

19. The host cell of claim 18, wherein the polynucleotide is expressed to produce the encoded polypeptide.

20. A method for assaying the effects of test compounds on the activity of a RGS9-evectin polypeptide dimer comprising the steps of:
(a) providing recombinant cells comprising a RGS9 polypeptide having an amino acid sequence of SEQ ID NO:2 and an evectin polypeptide having an amino acid sequence of SEQ ID NO:4;
(b) contacting the cells with a test compound; and
(c) determining the effect of the test compound on the activity of the RGS9-evectin dimer in the presence and absence of the test compound.

21. The method of claim 20, wherein the activity of the RGS9-evectin polypeptide dimer is detected by a G-protein second messenger response selected from the group consisting of an inositol triphosphate/diacyl glycerol-protein kinase C system, an adenylate cyclase/cyclic AMP-dependent protein kinase system, a guanylate cyclase/cGMP dependent protein kinase system and an ion channel.

22. A method for assaying the effects of test compounds on the activity of a RGS9-evectin polypeptide dimer comprising the steps of:
(a) providing a transgenic animal comprising a polynucleotide encoding a RGS9 polypeptide having an amino acid sequence of SEQ ID NO:2 and a polynucleotide encoding an evectin polypeptide having an amino acid sequence of SEQ ID NO:4;
(b) administering a test compound to the animal; and
(c) determining the effects of the test compound on the activity of the RGS9-evectin polypeptide in the presence and absence of the test compound.

23. A method for assaying the effects of test compounds on a transgenic animal with a genome comprising a functional disruption of the evectin binding domain in the RGS9 polypeptide, the method comprising:
(a) providing a transgenic animal whose genome comprises a disruption of the endogenous polynucleotide encoding the RGS9 polypeptide, wherein the disruption occurs in the evectin binding domain;
(b) administering a test compound to the animal; and
(c) determining the effects of the test compound on the activity of the RGS9 polypeptide in the presence and absence of the test compound.

24. A method for assaying the effects of test compounds on a transgenic animal with a genome comprising a functional disruption of the RGS9 binding domain in the evectin polypeptide, the method comprising:
(b) providing a transgenic animal whose genome comprises a disruption of the endogenous polynucleotide encoding the evectin polypeptide, wherein the disruption occurs in the RGS9 binding domain;
(b) administering a test compound to the animal; and
(c) determining the effects of the test compound on the activity of the evectin polypeptide in the presence and absence of the test compound.

25. A method for assaying the effects of test compounds on the binding interaction of RGS9 and evectin polypeptides comprising the steps of:
(a) providing yeast cells for a yeast two-hybrid system comprising a RGS9 polypeptide having an amino acid sequence of SEQ ID NO:2 and an evectin polypeptide having an amino acid sequence of SEQ ID NO:4;
(b) contacting the cells with a test compound; and
(c) determining the effect of the test compound on the binding interaction of the RGS9 and evectin polypeptides in the presence and absence of the test compound.

26. A method for modulating G-protein activity in a subject comprising administering to the subject a therapeutically effective amount of the polypeptide according to claims 1, 2 or 5.

27. A method for modulating G-protein activity in a subject comprising administering to the subject a therapeutically effective amount of a polynucleotide antisense to the polynucleotide according to claims 3, 4 or 7.

28. A method for modulating G-protein activity in a subject comprising administering to the subject a therapeutically effective amount an antibody according to claims 8, 9 or 10.

29. A method for modulating G-protein activity in a subject comprising administering to the subject a therapeutically effective amount an expression vector according to claims 15, 16 or 17.

30. A method for the diagnosis of a disease or susceptibility to a disease in a subject related to the activity of a RGS9-evectin dimer, the method comprising:
(a) obtaining a biological sample from the subject;
(b) contacting the sample with an oligonucleotide probe of a polynucleotide encoding the RGS9 polypeptide fragment of claim 1 and an oligonucleotide probe of a polynucleotide encoding the evectin polypeptide fragment of claim 2, under stringent hybridization conditions;
(c) isolating the hybrids from the sample; and
(d) sequencing the hybrids; wherein a mutation in the RGS9 and/or evectin polynucleotide sequence indicates a disease or susceptibility to a disease related to the activity of a RGS9-evectin dimer.

31. A method for the diagnosis of a disease or susceptibility to a disease in a subject related to the activity of a RGS9-evectin dimer, the method comprising:
(a) obtaining a biological sample from the subject;
(b) contacting the sample with an oligonucleotide primer of a polynucleotide encoding the RGS9 polypeptide fragment of claim 1 and an oligonucleotide primer of a polynucleotide encoding the evectin polypeptide fragment of claim 2, in the presence of nucleotides and a polymerase enzyme under conditions permitting primer extension;
(d) isolating primer extension products in the sample, and
(e) sequencing the primer extension products; wherein a mutation in the RGS9 and/or evectin polynucleotide sequence indicates a disease or susceptibility to a disease related to the activity of a RGS9-evectin dimer.

32. The method according to claims 30 or 31, wherein the disease is a neurological disorder.

33. A method for the treatment of a subject in need of inhibiting RGS9-evectin dimer activity comprising:
(a) administering to the subject a therapeutically effective amount of an antagonist to the RGS9-evectin; or
(b) administering to the subject a polynucleotide that inhibits the expression of a polynucleotide encoding a RGS9-evectin polypeptide; or
(c) administering to the subject a therapeutically effective amount of a polypeptide that competes with RGS9-evectin for its ligand.

34. A method for the treatment of a subject in need of enhanced RGS9-evectin dimer activity comprising:
(a) administering to the subject a therapeutically effective amount of an agonist to the RGS9-evectin; or
(b) administering to the subject a polynucleotide encoding a RGS9-evectin polypeptide, in a form so as to effect the production of the RGS9-evectin activity in vivo.

35. A method for producing a transgenic animal whose genome comprises a functional disruption of the evectin binding domain in a polynucleotide encoding a RGS9 polypeptide, the method comprising:
(a) providing a polynucleotide encoding a RGS9 polypeptide having a functional disruption in the evectin binding domain, wherein the binding domain comprises the amino acid sequence of amino acid 461 through amino acid 602 of SEQ ID NO:2;
(b) introducing the disrupted polynucleotide into embryonic stem cells;
(c) selecting those embryonic stem cells that comprise the disrupted polynucleotide;
(d) introducing an embryonic stem cell of step (c) into a blastocyst;
transferring the blastocyst of step (d) to a pseudopregnant animal; and
(e) allowing the transferred blastocyst to develop into an animal chimeric for the disruption.

36. A method for producing a transgenic animal whose genome comprises a functional disruption of the RGS9 binding domain in a polynucleotide encoding an evectin polypeptide, the method comprising:
(a) providing a polynucleotide encoding an evectin polypeptide having a functional disruption in the RGS9 binding domain, wherein the binding domain comprises the amino acid sequence of amino acid 79 through amino acid 136 of SEQ ID NO:4;
(b) introducing the disrupted polynucleotide into embryonic stem cells;
(c) selecting those embryonic stem cells that comprise the disrupted polynucleotide;
(d) introducing an embryonic stem cell of step (c) into a blastocyst; transferring the blastocyst of step (d) to a pseudopregnant animal; and
(e) allowing the transferred blastocyst to develop into an animal chimeric for the disruption.
